SEQUENCE LISTING (37 sequences)

SEQ ID NO: 1 - protein, 309 aa, Homo sapiens
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 2 - protein, 6 aa, synthetic construct
  REGION 1..6: note = alpha chain CDR 1 of TCR T1367
DSSSTY

SEQ ID NO: 3 - protein, 7 aa, synthetic construct
  REGION 1..7: note = alpha chain CDR 2 of TCR T1367
IFSNMDM

SEQ ID NO: 4 - protein, 15 aa, synthetic construct
  REGION 1..15: note = alpha chain CDR 3 of TCR T1367
CAESIGSNSGYALNF

SEQ ID NO: 5 - protein, 5 aa, synthetic construct
  REGION 1..5: note = beta chain CDR 1 of TCR T1367
MDHEN

SEQ ID NO: 6 - protein, 6 aa, synthetic construct
  REGION 1..6: note = beta chain CDR 2 of TCR T1367
SYDVKM

SEQ ID NO: 7 - protein, 13 aa, synthetic construct
  REGION 1..13: note = beta chain CDR 3 of TCR T1367
CASRGLAGYEQYF

SEQ ID NO: 8 - protein, 6 aa, synthetic construct
  REGION 1..6: note = alpha chain CDR 1 of TCR T1405
DSASNY

SEQ ID NO: 9 - protein, 7 aa, synthetic construct
  REGION 1..7: note = alpha chain CDR 2 of TCR T1405
IRSNVGE

SEQ ID NO: 10 - protein, 14 aa, synthetic construct
  REGION 1..14: note = alpha chain CDR 3 of TCR T1405
CAARPNSGNTPLVF

SEQ ID NO: 11 - protein, 5 aa, synthetic construct
  REGION 1..5: note = beta chain CDR 1 of TCR T1405
SQVTM

SEQ ID NO: 12 - protein, 7 aa, synthetic construct
  REGION 1..7: note = beta chain CDR 2 of TCR T1405
ANQGSEA

SEQ ID NO: 13 - protein, 14 aa, synthetic construct
  REGION 1..14: note = beta chain CDR 3 of TCR T1405
CSVEQDTNTGELFF

SEQ ID NO: 14 - protein, 6 aa, synthetic construct
  REGION 1..6: note = alpha chain CDR 1 of TCR T1705
NSAFQY

SEQ ID NO: 15 - protein, 6 aa, synthetic construct
  REGION 1..6: note = alpha chain CDR 2 of TCR T1705
TYSSGN

SEQ ID NO: 16 - protein, 12 aa, synthetic construct
  REGION 1..12: note = alpha chain CDR 2 of TCR T1705
CAMSDTGNQFYF

SEQ ID NO: 17 - protein, 5 aa, synthetic construct
  REGION 1..5: note = beta chain CDR 1 of TCR T1705
PRHDT

SEQ ID NO: 18 - protein, 6 aa, synthetic construct
  REGION 1..6: note = beta chain CDR 2 of TCR T1705
FYEKMQ

SEQ ID NO: 19 - protein, 15 aa, synthetic construct
  REGION 1..15: note = beta chain CDR 3 of TCR T1705
CASSFRGGGANVLTF

SEQ ID NO: 20 - protein, 7 aa, synthetic construct
  REGION 1..7: note = alpha chain CDR 1 of TCR R37P1C9
TISGTDY

SEQ ID NO: 21 - skipped

SEQ ID NO: 22 - protein, 11 aa, synthetic construct
  REGION 1..11: note = alpha chain CDR 3 of TCR R37P1C9
CILFNFNKFYF

SEQ ID NO: 23 - protein, 5 aa, synthetic construct
  REGION 1..5: note = beta chain CDR 1 of TCR R37P1C9
LNHNV

SEQ ID NO: 24 - protein, 6 aa, synthetic construct
  REGION 1..6: note = beta chain CDR 2 of TCR R37P1C9
YYDKDF

SEQ ID NO: 25 - protein, 14 aa, synthetic construct
  REGION 1..14: note = beta chain CDR 3 of TCR R37P1C9
CATSSGETNEKLFF

SEQ ID NO: 26 - protein, 6 aa, synthetic construct
  REGION 1..6: note = alpha chain CDR 1 of TCR R35P3A4
DSASNY

SEQ ID NO: 27 - skipped

SEQ ID NO: 28 - protein, 14 aa, synthetic construct
  REGION 1..14: note = alpha chain CDR 3 of TCR R35P3A4
CAASPTGGYNKLIF

SEQ ID NO: 29 - protein, 5 aa, synthetic construct
  REGION 1..5: note = beta chain CDR 1 of TCR R35P3A4
MNHEY

SEQ ID NO: 30 - protein, 6 aa, synthetic construct
  REGION 1..6: note = beta chain CDR 2 of TCR R35P3A4
SVGAGI

SEQ ID NO: 31 - protein, 14 aa, synthetic construct
  REGION 1..14: note = beta chain CDR 3 of TCR R35P3A4
CASSLGGASQEQYF

SEQ ID NO: 32 - protein, 7 aa, synthetic construct
  REGION 1..7: note = alpha chain CDR 1 of TCR R37P1H1
TSESNYY

SEQ ID NO: 33 - protein, 4 aa, synthetic construct
  REGION 1..4: note = alpha chain CDR 2 of TCR R37P1H1
QEAY

SEQ ID NO: 34 - protein, 15 aa, synthetic construct
  REGION 1..15: note = alpha chain CDR 3 of TCR R37P1H1
CAFGYSGGGADGLTF

SEQ ID NO: 35 - protein, 5 aa, synthetic construct
  REGION 1..5: note = beta chain CDR 1 of TCR R37P1H1
SGHDT

SEQ ID NO: 36 - protein, 6 aa, synthetic construct
  REGION 1..6: note = beta chain CDR 2 of TCR R37P1H1
YYEEEE

SEQ ID NO: 37 - protein, 16 aa, synthetic construct
  REGION 1..16: note = beta chain CDR 3 of TCR R37P1H1
CASSNEGQGWEAEAFF

(19) United States
(12) Patent Application Publication
(10) Pub. No.: US 2023/0310607 A1
(43) Pub. Date: Oct. 5, 2023

(54) METHODS OF SELECTING A PATIENT FOR TREATMENT OF A MAGE-A1 POSITIVE SOLID TUMOR, OF PREDICTING WHETHER A PATIENT BEING DIAGNOSED WITH MAGE-A1 POSITIVE SOLID TUMOR WILL BE RESPONSIVE TO TREATMENT OF THIS TUMOR AND OF TREATING A PATIENT BEING DIAGNOSED WITH SUCH A MAGE-A1 POSITIVE SOLID TUMOR AS WELL AS CORRESPONDING PHARMACEUTICAL COMPOSITIONS AND DIAGNOSTIC KITS

(71) Applicant: T-Knife GmbH, Berlin (DE)

(72) Inventor: Eugen LEO, Berlin (DE)

(21) Appl. No.: 18/054,085

(22) Filed: Nov. 9, 2022

(30) Foreign Application Priority Data
Nov. 9, 2021 (EP) 21207332.4

(51) Int. Cl. A61K 39/00; G01N 33/574

(52) U.S. Cl. CPC A61K 39/464486; G01N 33/57496; A61K 39/464411; A61K 39/4622; A61K 39/4611; A61K 39/4631; A61K 39/4634; A61K 39/4632; G01N 2800/52; G01N 2474/20

(57) ABSTRACT

The present invention inter alia relates to a method of selecting a patient for treatment of a solid tumor, wherein cells of the tumor express the human melanoma associated antigen 1, to a method of predicting whether a patient being diagnosed with a MAGE-1A positive solid tumor will be responsive to treatment of this tumor as well as to methods of treating a patient being diagnosed with a MAGE-1A solid tumor. The invention also relates to a pharmaceutical composition comprising T cells expressing a T cell receptor that specifically binds MAGE-1A and to a diagnostic immunostaining kit for selecting a patient for treatment of a solid tumor.

### SEQUENCE LISTING

The instant application contains a Sequence Listing which has been submitted electronically in XML format and is hereby incorporated by reference in its entirety. Said XML copy, created on Nov. 9, 2022, is named 51600-007002_Sequence_Listing_11_9_22 and is 45,354 bytes in size.

### CROSS-REFERENCE TO RELATED APPLICATIONS

The present application claims the benefit of priority of European Patent Application No. 21207332.4, filed Nov. 9, 2021, the content of which is hereby incorporated by reference it its entirety for all purposes.

### FIELD OF THE INVENTION

The present invention relates to a method of selecting a patient for treatment of a solid tumor, wherein cells of the tumor express human melanoma associated antigen 1 (MAGE-A1), to a method of predicting whether a patient being diagnosed with such a MAGE-A1 positive solid tumor will be responsive to treatment of this tumor as well as to methods of treating a patient being diagnosed with such a MAGE-A1 positive solid tumor. The invention also relates to a pharmaceutical composition comprising T cells expressing a T cell receptor (TCR) that specifically binds MAGE-A1 and a diagnostic immunostaining kit for selecting a patient for treatment of a solid tumor, wherein cells of the tumor express the human melanoma associated antigen 1 (MAGE-A1). The invention also relates to an adoptive cell therapy agent specifically binding MAGE-A1 or an agent specifically binding MAGE-A1 for use in treating a patient being diagnosed with a solid tumor, wherein cells of the tumor express human melanoma associated antigen 1 (MAGE-A1), wherein a patient is selected for treatment if a fraction of at least 30% of the cells of a tumor sample obtained from the patient are found to express MAGE-A1.

### BACKGROUND OF THE INVENTION

The melanoma antigen genes (MAGE-A) were found to be expressed in a variety of tumors of different histological origin. Proteins encoded by the MAGE genes are tumor rejection antigens, which can induce specific cytotoxic T-lymphocytes (CTL) having the ability to recognize and kill cancerous cells. MAGE genes and proteins are thus a promising target for development of drugs to fight cancer by immunotherapy. MAGE-A proteins constitute a sub-family of Cancer-Testis Antigens which while being expressed in the germ line, they are also expressed in various human cancers where they are associated with, and may drive, malignancy. This specific expression of MAGE antigens in tumors and not the normal surrounding healthy tissue makes this family of antigens very interesting for targeted adoptive T cell transfer. The treatment of solid tumors by means of genetically modified T-cells expressing a TCR recognizing a MAGE antigen has attracted particular interest. However, the suitability of existing protocols for patient selection is not yet known. There is thus a need for novel methodologies for selecting patients for treatment of MAGE-A1 positive cancers. Accordingly, it is an object of the invention to provide such methodologies.

### SUMMARY OF THE INVENTION

This object is inter alia accomplished by the methods, compositions, and kits having the features of the respective independent claims.

In a first aspect, the invention provides a method of selecting a patient for treatment of a solid tumor, wherein cells of the tumor express human melanoma associated antigen 1 (MAGE-A1) (e.g., UniProtKB accession number P43355 (MAGA1_HUMAN), the method comprising determining in a tumor cell sample obtained from the patient the fraction of cells that express MAGE-A1, wherein a patient is selected for treatment if a fraction of at least 30% of the cells of the tumor sample (e.g., 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, of the cells of the tumor sample) are found to express MAGE-A1.

In a second aspect, the invention provides a method of predicting whether a patient being diagnosed with a solid tumor will be responsive to treatment of this tumor, wherein cells of the tumor express human melanoma associated antigen 1 (MAGE-A1), the method comprising determining in a tumor cell sample obtained from the patient the fraction of cells that express MAGE-A1, wherein a patient is selected for/determined to be responsive to treatment if a fraction of at least 30% of the cells of the tumor sample (e.g., 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, of the cells of the tumor sample) are found to express MAGE-A1.

In a third aspect, the invention provides a method of treating a patient being diagnosed with a solid tumor, wherein cells of the tumor express human melanoma associated antigen 1 (MAGE-A1), wherein a patient is selected for treatment if a fraction of at least 30% of the cells of a tumor cell sample (e.g., 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, of the cells of the tumor sample) obtained from the patient are found to express MAGE-A1, wherein the method comprises administering to the patient a therapeutically effective amount of an adoptive cell therapy agent or an agent specifically binding MAGE-A1.

In a fourth aspect, the invention provides a method of treating a patient having a solid tumor, wherein a fraction of at least 30% of cells of a sample of the tumor obtained from the patient (e.g., 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, of the cells) has been determined to express MAGE-A1, the method comprising administering to the patient a therapeutically effective amount of an adoptive cell therapy agent or an agent specifically binding MAGE-A1.

In a fifth aspect, the invention provides a method of treating a patient having a solid tumor, the method comprising:
a) determining that at least 30% of cells of a sample of the tumor obtained from the patient (e.g., 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, of the cells) express MAGE-A1; and
b) administering to the patient a therapeutically effective amount of an adoptive cell therapy agent or an agent specifically binding MAGE-A1.

In a sixth aspect the invention provides a pharmaceutical composition comprising T cells expressing a T cell receptor that specifically binds MAGE-A1, wherein the T-cell receptor comprises
an alpha chain comprising the CDR sequences shown in SEQ ID NO: 2 (Ile Phe Ser Asn Met Asp Met), SEQ ID NO: 3 (Ile Phe Ser Asn Met Asp Met) and SEQ ID NO: 4 (Cys Ala Glu Ser Ile Gly Ser Asn Ser Gly Tyr Ala Leu Asn Phe); and a beta chain comprising the CDR sequences shown in SEQ ID NO: 5 (Met Asp His Glu Asn), SEQ ID NO: 6 (Ser Tyr Asp Val Lys Met), and SEQ ID NO: 7 (Cys Ala Ser Arg Gly Leu Ala Gly Tyr Glu Gln Tyr Phe);
an alpha chain comprising the CDR sequences shown in SEQ ID NO: 8 (Asp Ser Ala Ser Asn Tyr), SEQ ID NO: 9 (Ile Arg Ser Asn Val Gly Glu), and SEQ ID NO: 10 (Cys Ala Ala Arg Pro Asn Ser Gly Asn Thr Pro Leu Val Phe); and a beta chain comprising the CDR sequences shown in SEQ ID NO: 11 (Ser Gln Val Thr Met), SEQ ID NO: 12 (Ala Asn Gln Gly Ser Glu Ala), and 13 (Cys Ser Val Glu Gln Asp Thr Asn Thr Gly Glu Leu Phe Phe); or
an alpha chain comprising the CDR sequences shown in SEQ ID NO: 14 (Asn Ser Ala Phe Gln Tyr), SEQ ID NO: 15 (Thr Tyr Ser Ser Gly Asn), and SEQ ID NO: 16 (Cys Ala Met Ser Asp Thr Gly Asn Gln Phe Tyr Phe); and a beta chain comprising the CDR sequences shown in SEQ ID NO: 17 (Pro Arg His Asp Thr), SEQ ID NO: 18 (Phe Tyr Glu Lys Met Gln), and SEQ ID NO: 19 (Cys Ala Ser Ser Phe Arg Gly Gly Gly Ala Asn Val Leu Thr Phe); and
   wherein the total number of T cells comprised in the composition is from about 0.5×10⁷ T cells to about 1×10¹⁰ T cells. In some embodiments of this aspect, the total number of T cells comprised in the composition is from 0.5×10⁷ T cells to 1×10¹⁰ T cells.

In a seventh aspect, the invention provides a diagnostic immunostaining kit for selecting a patient for treatment of a solid tumor, wherein cells of the tumor express the human melanoma associated antigen 1 (MAGE-A1) (e.g., UniProtKB accession number P43355 (MAGA1_HUMAN), wherein the kit comprises
the monoclonal IgG1 mouse antibody MA454 or a fragment of the antibody MA454, and
a secondary antibody capable of binding to the monoclonal IgG1 mouse antibody MA454.

In a related eighth aspect, the invention provides the use of the monoclonal IgG1 mouse antibody MA454 or an antigen binding fragment of the antibody MA454 for selecting a patient for treatment of a solid tumor, wherein cells of the tumor express human melanoma associated antigen 1 (MAGE-A1) (e.g., UniProtKB accession number P43355 (MAGA1_HUMAN).

In a related nineth aspect the invention provides a method of selecting a patient for treatment of a solid tumor, wherein cells of the tumor express human melanoma associated antigen 1 (MAGE-A1) (e.g., UniProtKB accession number P43355 (MAGA1_HUMAN), wherein the method comprises contacting a tumor cell sample obtained from the patient with the monoclonal IgG1 mouse antibody MA454 or an antigen binding fragment of the antibody MA454. An embodiment of this aspect comprises determining in the tumor cell sample obtained from the patient the fraction of cells that express MAGE-A1. In this embodiment, a patient may be selected for treatment if a fraction of at least 30% of the cells of the tumor sample are found to express MAGE-A1.

### BRIEF DESCRIPTION OF THE DRAWINGS

The application file contains at least one drawing executed in color. Copies of this patent or patent application with color drawings will be provided by the Office upon request and payment of the necessary fee.

The invention will be better understood with reference to the detailed description when considered in conjunction with the non-limiting examples and the drawings, in which:

FIG. 1 shows examples of MAGE-A1 scores in tumor samples that also contain non-tumoral tissue, with FIG. 1a showing negative (0%), FIG. 1b showing 10%, FIG. 1c showing 20%, FIG. 1d showing 40%, FIG. 1e showing 60%, FIG. 1f showing 80%, FIG. 1g showing 90% and FIG. 1h) showing 100% positive tumor cells.

FIG. 2 shows examples of MAGE-A1 (antibody MA454) immunostaining in cell lines and tissues. FIG. 2a shows 293T-MAGE A1 cells with positive staining. The different staining intensities in individual cells result from variable amounts of MAGE-A1 expression. FIG. 2b shows as a negative control untransfected 293T cells. FIG. 2c shows as a positive control human testis tissue.

FIG. 3 shows examples of MAGE-A1 staining in normal tissues. FIG. 3a shows staining in cerebellum cortex, FIG. 3b shows staining in cerebellum grey matter, FIG. 3c shows staining in cerebrum grey matter, FIG. 3d shows staining in cerebrum white matter, FIG. 3e shows staining in colon mucosa, FIG. 3f shows staining in heart, FIG. 3g shows staining in kidney cortex, FIG. 3h shows staining in liver, FIG. 3i shows staining in lung, FIG. 3j shows staining in small intestine, FIG. 3k shows staining in stomach corpus, and FIG. 3l shows staining in testis.

FIG. 4 shows examples of MAGE-A1 positive and negative tumors. FIG. 4a shows muscle invasive urinary bladder cancer (100% pos. tumor cells), FIG. 4b shows squamous cell carcinoma of the skin (10%), FIG. 4c shows malignant melanoma (100%), FIG. 4d shows malignant melanoma (60%), FIG. 4e shows pancreatic adenocarcinoma (negative), and FIG. 4f shows clear cell renal cell carcinoma (negative).

FIG. 5 shows staining results of patient #62 showing spots 1 and 3 with 100% positive tumor cells but negative staining in spots 2, 4 and 5.

FIG. 6 shows a schematic representation of the MAGE-A1 staining results in 105 bladder cancers with multiple (Spot 1-5) analyzed samples. Boxes represent individual tissue spots. Color indicates the percentage of stained tumor cells. The one heterogeneous cancer (Pat. 62) is marked by a bold box.

FIG. 7 shows staining results for patient #232: MAGE-A1 negative primary tumor (left) and MAGE-A1 positive (60% of tumor cells) lymph node metastasis (right).

FIG. 8 shows a schematic representation of the MAGE-A1 staining results in 342 esophageal cancers (Patients 1-342) with up to 3 samples from the primary tumor, lymph node metastasis (LK met) and distant metastasis. Boxes represent individual tissue spots. Color indicates the percentage of stained tumor cells. Heterogeneous cancers are marked by bold boxes. Gray boxes indicate non-interpretable samples; white boxes indicate that no corresponding sample was included in the TMA.

FIG. 9 shows MAGE-A1 staining in three primary cancer spots of patient 135 showing different percentages of MAGE-A1 positive tumor cells: left: negative, middle: 10%, right: 90%.

FIG. 10 shows MAGE-A1 staining in patient #39: pT1, pT3, pT4, pT6: Primary cancer spots with negative MAGE-A1 staining, LN1: Lymph node metastasis with negative MAGE-A1 staining, LN2: Different lymph node metastasis with strong MAGE-A1 staining in all (100%) tumor cells.

FIG. 11 shows a schematic representation of the MAGE-A1 staining results in 146 lung cancers (Patients 1-146) with up to 8 analyzed samples from the primary tumor (primary 1-8), and up to 4 lymph node metastases (LN met 1-4). Boxes represent individual tissue spots. Color indicates the percentage of stained tumor cells. Heterogeneous cancers are marked by bold boxes.

FIG. 12 shows MAGE-A1 staining in two primary cancer spots of patient 62 showing different percentages of MAGE-A1 positive tumor cells: left: 20%, right: negative.

FIG. 13 shows a schematic representation of the MAGE-A1 staining results in 113 stomach cancers (Pat. 1-113) with 9 samples from the primary tumor (primary 1-9), and 3 spots each from up to 3 lymph node metastases (LN 1-3). Boxes represent individual tissue spots. Color indicates the percentage of stained tumor cells. The two heterogeneous cancers (patients 17 and 62) are marked by a bold box.

FIG. 14 shows a flowchart of steps of an illustrative example of the methods described herein. Patients diagnosed with a solid tumor who are considered to receive treatment, for example, with an adoptive cell therapy agent such as chimeric antigen receptor T-cell (CAR T-cell) or a genetically modified T-cell that expresses a T cell receptor that specifically binds MAGE 1A are subjected to an eligibility test by determining their HLA genotype (HLA-A*02:01 in this illustrative example) and determining the availability of a tumor cell sample of the solid tumor. If the patient is HLA-A*02:01 positive and a tumor cell sample can be provided, the fraction of the cells of the tumor cell sample that is found to express MAGE-A1 is determined. In case the fraction of MAGE-A1 positive cells meets or exceeds a predetermined threshold, the patient will be selected for treatment of the solid tumor.

### DETAILED DESCRIPTION OF THE INVENTION

As explained above, in a first aspect the invention is directed to a method of selecting a patient for treatment of a solid tumor, wherein cells of the tumor express human melanoma associated antigen 1 (MAGE-A1) (UniProtKB accession number P43355 (MAGA1_HUMAN), the method comprising determining in a tumor cell sample obtained from the patient the fraction of cells that express MAGE-A1, wherein a patient is selected for treatment if a fraction of at least 30% of the cells of the tumor sample are found to express MAGE-A1. The use of such a threshold value has not yet been reported for the selection of patients for the treatment of solid tumors which express human melanoma associated antigen 1 (MAGE-A1). These tumors will be also referred to herein as “MAGE-A1 positive tumors”. It is noted here that a threshold value as an inclusion criterion for selecting patient for the treatment of MAGE-A1 positive has so far only been reported for a phase I clinical trial conducted at the Charité Campus Berlin Buch (Germany, German clinical trial registration number DRKS00020221) in which MAGE-A1-specific T-cell receptor (TCR)-transduced T cells are administered to patients with relapsed/refractory multiple myeloma.

It is noted in this context that is has been surprisingly found here that the monoclonal IgG1 mouse antibody MA454 (or an antigen binding fragment of the antibody MA454) is highly specific for MAGE-A1 and allows the determination of the percentage of MAGE-A1 expressing cells in tissue such as tumor cell samples from a wide range of different tumor cells. As found herein, this is further supported by analysis of normal (healthy) tissue, which revealed positive staining only in the expected tissue type (testis). It has also been found that the antibody MA454 does not show relevant non-specific background staining. These properties allow using a relatively high antibody concentration (for example, 8 μg/ml, see the Experimental Section) to detect MAGE-A1 with the highest possible sensitivity. This in turn allows using the antibody MA454 as a companion diagnostic for selecting a patient for treatment of a MAGE-1A positive solid tumor as well as for predicting whether a patient being diagnosed with a MAGE-A1 positive solid tumor will be responsive to treatment of this tumor.

When used herein, the term ‘human melanoma associated antigen 1 (MAGE-A1) is used in its regular meaning and refers to the protein the sequence of which is deposited in the UniProt database under UniProtKB accession number P43355 (MAGA1_HUMAN), its natural variants, allelic variants, splice isoforms thereof. Examples of such variants include the natural variants VAR_004283 in which the threonine residue at sequence positon 32 is replaced by alanine (T→A), the natural variant VAR_053491 in which the alanine residue at sequence position 63 is replaced by threonine (A→T), the natural variant VAR_011737 in which the arginine residue at sequence position 72 is replaced by Glutamine (R→Q) or the natural variant VAR_036581 in which in a breast cancer sample the lysine residue at sequence position 278 is replaced by threonine due to a somatic mutation (K→T) (see in this respect, the UniProt database entry of MAGE-A1. The term “MAGE-A1” also includes the melanoma antigen family A1 (fragment) that has a sequence identity of more than 90% with MAGE-A1 and which is deposited under the accession number UniProtKB-A8IF97 (A8IF97_HUMAN).

Reverting to the aspects of the invention, in a further aspect the invention is directed to a method of predicting whether a patient being diagnosed with a solid tumor will be responsive to treatment of this tumor, wherein cells of the tumor express human melanoma associated antigen 1 (MAGE-A1), the method comprising determining in a tumor cell sample obtained from the patient the fraction of cells that express MAGE-A1, wherein a patient is selected for treatment/determined to be responsive to treatment if a fraction of at least 30% of the cells of the tumor sample are found to express MAGE-A1.

In yet a further aspect the invention is directed to a method of treating a patient being diagnosed with a solid tumor, wherein cells of the tumor express human melanoma associated antigen 1 (MAGE-A1), wherein a patient is selected for treatment if a fraction of at least 30% of the cells of a tumor cell sample obtained from the patient are found to express MAGE-A1, wherein the method comprises administering to the patient a therapeutically effective amount of an adoptive cell therapy agent or an agent specifically binding MAGE-A1.

In yet another further aspect, the invention is directed to a method of treating a patient having a solid tumor, wherein a fraction of at least 30% of cells of a sample of the tumor obtained from the patient has been determined to express MAGE-A1, the method comprising administering to the patient a therapeutically effective amount of an adoptive cell therapy agent or an agent specifically binding MAGE-A1.

In yet another further aspect, the invention is directed to a method of treating a patient having a solid tumor, the method comprising:
a) determining that at least 30% of cells of a sample of the tumor obtained from the patient express MAGE-A1; and
b) administering to the patient a therapeutically effective amount of an adoptive cell therapy agent or an agent specifically binding MAGE-A1.

The invention will be further explained in the following, making reference to either, several or all of these aspects. If reference is only made to one of these aspects, it is understood by the person skill in the art, that this reference nevertheless includes references to all other aspects of the invention, if applicable.

Dealing first with the solid tumor to be treated, the methods of the inventions can be applied to any solid tumor which has been found to express MAGE-A1 at a threshold level as described herein. This tumor can be a solid tumor that is already known to express MAGE-A1, but also any tumor for which it will be found that the tumor expresses MAGE-A1 at a given threshold as described herein. Accordingly, apart from solid tumors such as the ones that are expressly mentioned here, the present invention of selecting and subsequently treating patients addresses also solid tumors that have only a very small MAGE-A1 positive populations, as long as the population is found to express MAGE-1A at a threshold level as described here.

Types of Solid Tumors

Turning now to the solid tumor types in more detail, the patient may have, for example, been diagnosed with a genotype and/or advanced-stage metastatic solid tumor that express MAGE-A1. Examples of a solid tumor to be treated and thus the tumor cell sample that is obtained from the tumor may include, but are not limited to, melanoma, lung cancer, esophageal cancer, gastric cancer, breast cancer, ovarian cancer, mesothelioma cancer, bladder cancer, anal cancer, chondrosarcoma cancer, osteosarcoma cancer, sarcoma cancer, adenoma cancer, primitive neuroectodermal cancer (primitive neuroectodermal tumor (PNET), or combinations thereof, to mention only a few.

In illustrative embodiments of the solid tumor types mentioned above, the lung cancer may be, but is not limited to, non-small cell lung cancer (NSCLC), including squamous cell carcinoma of the lung, adenocarcinoma of the lung, large cell carcinoma of the lung and other histologic types of NSCLC) or small cell lung cancer, too ment. In other illustrative examples, the breast cancer may be, but is not limited to, ductal breast cancer, ductal-invasive breast cancer, invasive breast cancer, tubular breast cancer, medullary breast cancer or combinations thereof. In yet other illustrative examples, the gastric cancer may be gastric adenocarcinoma or squamous cell cancer. Turning to sarcoma cancer, the sarcoma cancer may be, but is not limited to, chondrosarcoma cancer, osteosarcoma cancer or combinations thereof. The adenoma cancer may include, but is also not limited to, gastric adenocarcinoma, pancreatic adenocarcinoma or combinations thereof.

Assays

The methods of the invention include the determination of the fraction of cells of the tumor sample that (are found to) express MAGE-A1. For this purpose, one or more tumor cell samples are obtained from a patient's solid tumor of interest. Such a tumor cell sample may be obtained by a biopsy that is taken form the patient's solid tumor of interest. Alternatively, the tumor cell sample may be obtained by surgery which is carried out to remove tumorous material. Once the tumor cell sample has been obtained, the fraction of cells of the tumor cell sample that express MAGE-A1 is determined. The fraction of cells of the tumor sample that are found to express MAGE-A1 can be determined by any method that is suitable to detecting MAGE-A1 in a tumor cell sample. Such a method can be an immunohistochemistry method such as immunostaining. The terms “immunohistochemistry” and “immunostaining”, respectively, are used herein in their regular meanings and refer to a process of selectively identifying a selected antigen, here MAGE-A1, in cells of a tissue sample by using the principle of binding reagents such as antibodies that bind specifically to the antigen of interest in the tumor cell or tissue sample, thereby allowing detection of the antigen such as MAGE-A1 here. Examples of immunostaining methods that can be used for detecting MAGE-A1 in the tumor cell sample and thus for determining the fraction of MAGE-A1 expressing cells include flow cytometry, western blotting, enzyme-linked immunosorbent assays (ELISA), (indirect) immunofluorescence or chromogenic (non-fluorescent) methods that use enzymes such as peroxidase or alkaline phosphatase. Such enzymes are capable of catalyzing reactions that yield a coloured product that is detectable by light microscopy. Alternatively, radioactive elements can be used as (radioactive) labels, and the immunoreaction can be visualized by autoradiography.

The immunostaining as carried out herein may include fixation of (parts of) the tumor cell sample. Fixation can be carried out using any known fixation protocol and it is within the average knowledge of the skilled artisan to empirically determine a suitable fixative and a corresponding fixation protocol. The tumor cell sample may be fixation using a standard fixative solution such as formalin (e.g. phosphate buffered formalin, unbuffered zinc formalin or alcoholic formalin), methanol or ethanol based fixative such as Methacarn or Clarke's solution, or proprietary fixative solutions such as “Fix-All” containing alcohol, barium chloride, and 10% formalin (available from Leica, Germany) or “O-Fix” containing alcohol and formalin (also available from Leica, Germany) to mention only a few. The fixated samples/specimen is then usually infiltrated with a liquid agent that can subsequently be converted into a solid that has appropriate physical properties and which will allow thin sections to be cut from it. This processing is also known to the skilled person as tissue embedding. A typical material that is also used herein is paraffin, yielding “paraffin sections” or “paraffin embedded samples”. Such paraffin embedded samples allow the use of a tissue microarray (TMA) technique (the person of average skill in the art knows that tissue microarrays are particularly useful in analysis of cancer/tumor samples) for the further processing and subsequent determination of the fraction of MAGE-A1 expressing cells in the tumor cell sample.

In the tissue microarray (TMA) technique, a hollow needle is used to remove tissue cores typically as small as 0.6 mm in diameter from regions of interest in paraffin-embedded tissues, here the clinical biopsies or samples of solid tumors of interest. These tissue cores are then inserted in a recipient paraffin block in a precisely spaced, array pattern. Sections from such a block are cut using a microtome, mounted on a microscope slide and then analyzed by any method of standard histological analysis (cf. the Experimental Section of the present application). Each microarray block can be cut into a large number of sections, for example 100 to 500 sections, which can be subjected to independent tests. Staining methodologies commonly employed in tissue microarray include immunohistochemistry, and fluorescent in situ hybridization.

In addition to immunostaining as described above, it is also possible to determine the expression of MAGE-1A in the tumor cell sample by means of flow cytometry, for example. For this purpose, the sample of the solid tumor can be processed for the flow cytometric analysis using published protocols such as the one described by Ferreira-Facio et al “Contribution of Multiparameter Flow Cytometry Immunophenotyping to the Diagnostic Screening and Classification of Pediatric Cancer”, PLOS ONE, March 2013, Volume 8, Issue 3, e5553 or ones referred to in the review of Pillai & Dorfman “Flow Cytometry of Nonhematopoietic Neoplasms” Acta Cytologica 2016; 60:336-343. In such a protocol, a sample of the solid tumor may be placed in a Petri dish with suitable buffer such as PBS, minced into small pieces with a scalpel blade and mechanically disaggregated, filtered to eliminate cell clumps and debris, centrifuged and resuspended in buffer and immediately stained for flow cytometry with a MAGE-A1 binding reagent such a detectably labelled anti-MAGE A1 antibody.

In accordance with the above, the detection and quantification of MAGE-1A expressing tumor cells by staining as described herein is typically carried out with an antibody molecule that specifically binds MAGE-A1 or with a proteinaceous binding molecule with antibody-like binding properties that specifically binds MAGE-A1. This antibody molecule or proteinaceous binding molecule with antibody-like binding properties can, for this purpose, be directly conjugated to/coupled with an optically detectable label such as an enzyme catalyzing a chromogenic reaction or a fluorescent label. Alternatively, the antibody molecule or proteinaceous binding molecule with antibody-like binding properties that specifically binds MAGE-A1 can serve as “primary binding reagent” (primary antibody”) and can be used together with a secondary binding reagent such as secondary antibody that comprises (usually be conjugation) an optically detectable label (cf. the Experimental Section in this regard), wherein the secondary binding reagent (secondary antibody) typically binds to structurally conserved regions of the primary binding reagent such as the constant domains of a full-length antibody or of an antigen binding fragment such as an Fab Fragment or an F(ab)₂′ fragment.

Any antibody molecule that specifically binds MAGE-A1 can be used herein, the antibody molecule can, for example, be a polyclonal antibody, a monoclonal antibody, or fragments typically derived from monoclonal antibodies, such as divalent antigen binding antibody fragments, or monovalent antigen binding antibody fragments. Examples of suitable divalent antibody fragments are (Fab)₂′-fragments, divalent single-chain Fv fragments or divalent single domain camelid antibodies (also knowns as nanobodies) that can be obtained by producing such single domain camelid antibodies as fusion proteins with a linker between the two single domain camelid antibodies. Examples of suitable monovalent antibody fragments include, but are not limited to an Fab fragment, an Fv fragment, or a single-chain Fv fragment (scFv).

In case monoclonal antibodies that specifically binds MAGE-A1 are used for the immunostaining as described herein, already known monoclonal antibodies can be used. It is, however, also possible to generate new antibodies by immunization or by evolutive methods such as phage-display. An example of an already known monoclonal antibody which has been found to be particularly suited for the methods described herein is the MAGE-A1 binding monoclonal IgG1 mouse antibody MA454 or an antigen binding fragment of the antibody MA454. The antibody MA454 raised against a partially purified, full length recombinant MAGE-A1 of human origin was first described by Chen et al Proc. Nati. Acad. Sci. USA Vol. 91, pp. 1004-1008, 1994. It is also described in U.S. Pat. No. 5,541,104 and International patent application WO 95/20974, and the hybridoma cell line that produces the antibody MA454 was deposited at the ATCC under accession number HB11540 (see WO 95/20974). Meanwhile the antibody MA454 is commercially available from various sources, for example from Abcam, Cambridge, UK under catalogue number ab193330, from Santa Cruz Biotechnology, Santa Cruz, CA, USA, under catalogue number sc-20033, from Origine Technologies, Inc. Rockville, MD USA under catalogue number AM32863PU-S or from Novus Biologicals, LLC, Centennial, CO, USA under catalogue number NBP2-33094R to name only a few commercial suppliers. It has been surprisingly found here that antibody MA454 (or an antigen binding fragment of the antibody MA454) is highly specific for MAGE-A1, allows the determination of the percentage of MAGE-A1 expressing cells in tissue such as tumor cell samples from a wide range of different tumor cells and does not show relevant unspecific background staining. These properties allow using a relatively high antibody concentration (for example, 8 μg/ml, see the Experimental Section) to detect MAGE-A1 with the highest possible sensitivity.

As mentioned above, as an alterative to antibody molecules, it is also possible to use for the immunostaining proteinaceous binding molecules with antibody-like binding properties. Such proteinaceous binding molecules with antibody-like binding properties are well-known to the person skilled in the art and have, for example, been reviewed by Skerra “Anticalins': a new class of engineered ligand-binding proteins with antibody-like properties” Rev. Mol. Biotechnol. 74, 257-275 (2001) or by Skerra “Engineered scaffolds for molecular recognition”, J Mol Recognit, 13:167-187 (2000), with the latter summarizing protein scaffolds that are being used for the generation of proteinaceous binding molecules with antibody-like binding properties.

In accordance with the above, examples of proteinaceous binding molecules with antibody-like binding properties that can be used herein include, but are not limited to, an aptamer, a mutein based on a polypeptide of the lipocalin family, a glubody, a protein based on the ankyrin scaffold, a protein based on the crystalline scaffold, an adnectin, an avimer, a EGF-like domain, a Kringle-domain, a fibronectin type I domain, a fibronectin type II domain, a fibronectin type III domain, a PAN domain, a G1 a domain, a SRCR domain, a Kunitz/Bovine pancreatic trypsin Inhibitor domain, tendamistat, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an immunoglobulin domain or a an immunoglobulin-like domain (for example, domain antibodies or camel heavy chain antibodies), a C-type lectin domain, a MAM domain, a von Willebrand factor type A domain, a Somatomedin B domain, a WAP-type four disulfide core domain, a F5/8 type C domain, a Hemopexin domain, an SH2 domain, an SH3 domain, a Laminin-type EGF-like domain, a C2 domain, “Kappabodies” (Ill. et al. “Design and construction of a hybrid immunoglobulin domain with properties of both heavy and light chain variable regions” Protein Eng 10:949-57 (1997)), “Minibodies” (Martin et al. “The affinity-selection of a minibody polypeptide inhibitor of human interleukin-6” EMBO J 13:5303-9 (1994)), “Janusins” (Traunecker et al. “Bispecific single chain molecules (Janusins) target cytotoxic lymphocytes on HIV infected cells” EMBO J 10:3655-3659 (1991) and Traunecker et al. “Janusin: new molecular design for bispecific reagents” Int J Cancer Suppl 7:51-52 (1992), an avimer (Silverman, Lu Q, Bakker A, To W, Duguay A, Alba B M, Smith R, Rivas A, Li P, Le H, Whitehorn E, Moore K W, Swimmer C, Perlroth V, Vogt M, Kolkman J, Stemmer W P 2005, Nat Biotech, December; 23(12):1556-61, E-Publication in Nat Biotech. 2005 Nov. 20 edition); as well as multivalent avimer proteins evolved by exon shuffling of a family of human receptor domains as also described in Silverman J, Lu Q, Bakker A, To W, Duguay A, Alba B M, Smith R, Rivas A, Li P, Le H, Whitehorn E, Moore K W, Swimmer C, Perlroth V, Vogt M, Kolkman J, Stemmer W P, Nat Biotech, December; 23(12):1556-61, E-Publication in Nat. Biotechnology. 2005 Nov. 20 edition

Patient Selection Criteria

Turning now in more detail to the threshold used in the methods described herein, a patient may be selected for treatment, if a fraction of least about 30% of the cells, at least about 40% of the cells, at least about 50% of the cells, of least about 60% of the cells, of least about 65%, least about 70% of the cells, of at least about 75% of the cells, of at least 80% of the cells, of at least about 90% of the cells, of at least about 95% of the cells, of at least about 96% of the cells, of at least about 97% of the cells, of at least about 98% of the cells, of at least about 99% of the cells or 100% of the cells of the tumor sample are found to express MAGE-A1. A patient may also be selected for treatment, if a fraction of least 30% of the cells, at least 40% of the cells, at least 50% of the cells, of least 60% of the cells, of least 65%, least 70% of the cells, of at least 75% of the cells, of at least 80% of the cells, of at least 90% of the cells, of at least 95% of the cells, of at least 96% of the cells, of at least 97% of the cells, of at least 98% of the cells, of at least 99% of the cells or 100% of the cells of the tumor sample are found to express MAGE-A1 A cell is considered to express MAGE-A1 when expression of MAGE-1A is indicated for this particular cell by the respective method, for example, if in case of immunostaining, a positive signal above the background level is obtained for this cell. It is noted here that as used herein with respect to the fraction of cells, the term “about” means to include a deviation from the respective value of up to 1%, of up to 2%, of up to 3%, of up to 4%, of up to 5%, or up to and including 10% of the given value. This means, for example, a fraction of “at about 70% of the cells” may include a fraction ranging from 70%±10%, i.e., from 63% to 77% (70%±7) of the cells are determined to express MAGE-A1.

In this context it is also noted that the fraction (percentage) of MAGE-A1 positive cells can be determined by any suitable method known to the person skilled in the art.

If, for example, a tissue microarray has been prepared from the solid tumor sample, sections from a block of the tissue microarray can be cut by means of a microtome, and then mounted on a microscope slide for the immunohistochemical staining and subsequent histological analysis. For example, for each tumor tissue spot analyzed, the staining intensity can be recorded in a step wise scale (for example, a four-step scale (0, 1+, 2+, 3+) and the total number of cells present in the tumor cell sample and the fraction of stained tumor cells can be determined visually. For example, a tumor can then be considered to express MAGE-A1 (meaning to be MAGE-A1 positive), if at least 5% of the tumor cells show immunostaining of intensity 1+ or more). By so doing, tumor spots can then be categorized according to the fraction of tumor cells with positive staining into a respective group, for example, of % positive tumor cells, of ≥50%. % positive tumor cells, of ≥60% positive tumor cells, of ≥70% positive tumor cells, of ≥80 (but <100%) positive tumor cells, and 100% positive tumor cells.

If instead of or in addition to the determination of the fraction of the MAGE-A1 positive cells by means of a TMA the tumor cell sample is analysed by flow cytometry, a signal intensity above a certain threshold (for example, higher than a negative control) can be set as being a MAGE-A1 positive cell and, so doing, the total number of cells present in the tumor cell sample and the fraction of stained tumor cells can be determined by flow cytometry.

The methods described herein can be applied to any solid tumor that either shows a homogenous or a heterogenous MAGE-A1 expression. In this context, a tumor is herein considered homogeneously positive with respect to MAGE-A1 expression when all cell samples taken from the same tumor have at least 5% MAGE-A1 positive cells. A solid tumor is herein considered MAGE-1A negative when all cell samples taken from the same tumor do not show expression of MAGE-1A. Finally, a tumor is herein considered heterogeneously positive with respect to MAGE-A1 expression when positive and negative results for MAGE-A1 expression are obtained in tumor cell samples taken from the same tumor. In illustrative terms, using tumor cell samples which are prepared and analyzed by means of a TMA, a tumor is considered i) homogeneously positive when all interpretable tissue spots have at least 5% MAGE-1A positive tumor cells, while it is considered ii) negative when all tissue spots have a negative immunohistochemistry result. In line with the above, a tumor is considered heterogeneously positive when positive and negative tissue spots are found to be present in the same tumor.

In this context, it is noted that for a tumor that is considered heterogeneously positive case, the same threshold can be applied as for a tumor that is MAGE-1A homogeneously positive. The only difference compared to MAGE-1A homogeneously positive tumors is that for selecting selecting patients for treatment of the solid tumors (or the method of predicting whether a patient being diagnosed with a solid tumor will be responsive to treatment of this tumor), it is sufficient that at least one of the tumor cell samples taken from the tumor is found to express MAGE-A1 in a fraction of about at least 30% of the cells of the tumor sample. As an illustrative example, if 10 tumor cell samples are taken from the tumor and only one of these tumor cell samples shows expression of MAGE-1A in say at least 30%, 40%, 50%, 60%, 70% or 80% of the cells the patient can be selected for treatment. Accordingly, a patient that is being diagnosed with such a MAGE-1A homogeneously positive tumor can be selected for treatment if a fraction of at least about 50% of the cells, of least about 60% of the cells, of least about 65%, least about 70% of the cells, of at least about 75% of the cells, of at least about 80% of the cells, of at least about 95% of the cells, of at least about 96% of the cells, of at least about 97% of the cells, of at least about 98% of the cells, of at least about 99% of the cells or 100% of the cells of at least one of the cell samples taken from tumor are found to express MAGE-A1. In other illustrative embodiments a patient that is being diagnosed with such a MAGE-1A homogeneously positive tumor can be selected for treatment if a fraction of at least 30% of the cells, of at least 40% of the cells, of at least 50% of the cells, of least 60% of the cells, of at least 65%, at least 70% of the cells, of at least 75% of the cells, of at least 80% of the cells, of at least 95% of the cells, of at least 96% of the cells, of at least 97% of the cells, of at least 98% of the cells, of at least 99% of the cells or 100% of the cells of at least one of the cell samples taken from tumor are found to express MAGE-A1.

Addressing in this context the treatment of the solid tumor or the eligibility of a cancer patient for treatment with a MAGE-A1 binding therapeutic agent/product as described herein, the MAGE-1A expression level can be determined at any suitable point of time prior to the intended treatment. For example, the tumor cell sample (biopsy) can be obtained from the patient in a first screening step several months or weeks prior to the intended treatment to see whether the patient is eligible for treatment. If in this screening step, the tumor cell sample(s) of the patient are found to exhibit a MAGE-A1 expression level that exceeds a threshold value as described here (of, for example, about 50% of the cells, of least about 60% of the cells, of least about 65%, least about 70% of the cells, of at least about 75% of the cells, of at least about 80% of the cells, or of at least about 85% of the cells are found to express MAGE-A1), then the patient is considered eligible for the treatment. At that point of time, the time point of the treatment, for example, administration of the cellular therapy agent, can already be chosen. In the case, that the treatment is an approved first line treatment, the patient will typically be treated as quickly as possible after being diagnosed to be eligible for the treatment. In case the therapy agent has been approved by a regulatory agency as second or third line treatment only, the treatment will typically start later after the first-line treatment. The exact time point of the treatment also usually depends on practical circumstances such as time to manufacture the cellular therapy product, if for example, autologous T cells derived from the patient are used for the treatment, or availability of hospital beds. If the therapeutic agent is a biological molecule such as an antibody-drug-conjugate (ADC) which is available “off the shelf” then treatment may start earlier since this agent does not need to be specifically manufactured for the treatment of the patient. After the initial diagnosis has been made, it is possible to confirm the expression level of MAGE-A1 by the tumor and so the eligibility of the patient for a treatment as described herein by means of a second biopsy that is taken at a suitable time after the initial diagnosis but before the scheduled treatment. For example, such a second test can be done 4 week after the initial diagnosis and 2 weeks prior to the scheduled time point of treatment, if for example, the patient is to be treated six weeks after it has been determined that the patient is eligible for a treatment as described herein.

Therapeutic Agents

Addressing now in more detail the treatment of the solid tumor, the treatment of the MAGE-1A positive solid tumor can be any suitable cancer treatment, for example, a treatment with a small molecule chemotherapeutic drug or immunotherapy. The term “immunotherapy” is used herein in its regular meaning to refer to a treatment that helps the immune system of a subject such as a human to fight a tumor disease or cancer. Accordingly, the term “immunotherapy” includes, for example, administration of immune checkpoint inhibitors, i.e., compounds such as small molecules or antibody molecules that that block immune checkpoints. These checkpoints are a normal part of the immune system and keep immune responses from being too strong. By blocking such checkpoints, immune checkpoint inhibitors such as small molecules or antibody molecules drugs allow immune cells to respond more strongly to the tumor. “Immunotherapy” as used herein also includes the use of specific binding proteins such as monoclonal antibodies or artificial binding molecules with antibody-like properties that are able to bind to specific surface molecules (targets) on tumor cells, so that the tumor cells will be better recognized and destroyed by the immune system. The term “immunotherapy” as used herein also includes a treatment that uses certain parts of a person's immune system to fight cancer/a solid tumor as described herein. Such an immunotherapy treatment, may, for example, include administration of autologous or allogeneic cells body cells such as white blood cells (lymphocytes). This form of treatment that uses cells of the immune system to treat tumor diseases (cancer) is also known as adoptive cell therapy or cellular immunotherapy. As mentioned above, examples of cells of the immune system that can be used for cellular immunotherapy are white blood cells. Such white blood cells include T cells such as CD8+ T cells or CD4+ T cells, or NK cells. Accordingly, the (cellular) immunotherapy may be T cell therapy (autologous or allogeneic), NK cell therapy (autologous or allogeneic), CAR-T cell therapy (autologous or allogeneic) or CAR-NK cell therapy (autologous or allogeneic) and the like.

In line with the above disclosure, the term “cell therapy agent” means a cellular agent that is used for immunotherapy. If such an agent is used for adoptive cell therapy, this agent is also referred herein as “adoptive cell therapy agent”. Examples of cell therapy agents may be genetically modified cells of the immune systems such as T cells or natural killer (NK) cells. Such genetically modified cells include chimeric antigen receptor T-cells (CAR T-cells, see Jakobsen & Gjerstorff “CAR T-Cell Cancer Therapy Targeting Surface Cancer/Testis Antigens”, Front. Immunol. 2 Sep. 2020, Article 01568, doi: 10.3389/fimmu.2020.01568 or genetically modified T-cells that expresses a T cell receptor that specifically binds MAGE 1A. The genetically modified cells may be autologous cells derived from the patient to be treated but also allogeneic cells, i.e., cells that are obtained not from the patient of interest but cells that have been derived from a “universal” donor cell. This “universal” donor cell may be derived from a naturally occurring cells such T cells or NK cells from a human donor (cf, in this respect, for example, the review article of Perez et al “Off-the-Shelf Allogeneic T Cell Therapies for Cancer: Opportunities and Challenges Using Naturally Occurring “Universal” Donor T Cells”, Front. Immunol., 11 Nov. 2020, Article 583716, https://doi.org/10.3389/fimmu.2020.583716.) It is however also possible to derive such a “universal” donor cells (T cell or NK cells) from induced pluripotent stem cells (iPSC), cf, the review article of Flahou et al, “Fit-For-All iPSC-Derived Cell Therapies and Their Evaluation in Humanized Mice With NK Cell Immunity” Front. Immunol., 2 Apr. 2021, Article 662360, https://doi.org/10.3389/fimmu.2021.662360). In case T genetically modified T cells are used as cell therapy agent, the T cells may be any suitable phenotype for example but not limited to CD8+ T cells, CD4+ T cells or a combination thereof. Regardless of whether autologous (patient derived) T cells are or allogeneic T cells are used a cell therapy agent are used, the T cell cells may express a recombinant T cell receptor (TCR) that specifically binds MAGE 1A. Suitable TCR that can be used herein are known and include, for example, the TCRs described in International Patent Application WO 2014/118236, (or corresponding issued patents such as U.S. Pat. No. 10,377,808 or EP patent 2 951 202), International Patent Application WO 2018/104438 or the corresponding U.S. Pat. No. 10,874,731), International Patent Application WO2018/170338, or International Patent Application WO2020/201318A1 or in Bassan et al, “Avidity optimization of a MAGE-A1-specific TCR with somatic hypermutation”, Eur. J. Immunol. 2021. 51: 1505-1518.

In illustrative examples the T cell receptor may comprise
an alpha chain comprising the CDR sequences shown in SEQ ID NO: 2 (Ile Phe Ser Asn Met Asp Met), SEQ ID NO: 3 (Ile Phe Ser Asn Met Asp Met) and SEQ ID NO: 4 (Cys Ala Glu Ser Ile Gly Ser Asn Ser Gly Tyr Ala Leu Asn Phe); and a beta chain comprising the CDR sequences shown in SEQ ID NO: 5 (Met Asp His Glu Asn), SEQ ID NO: 6 (Ser Tyr Asp Val Lys Met), and SEQ ID NO: 7 (Cys Ala Ser Arg Gly Leu Ala Gly Tyr Glu Gln Tyr Phe) as the TCR T1367 described in International Patent Application WO 2014/118236,
an alpha chain comprising the CDR sequences shown in SEQ ID NO: 8 (Asp Ser Ala Ser Asn Tyr), SEQ ID NO: 9 (Ile Arg Ser Asn Val Gly Glu), and SEQ ID NO: 10 (Cys Ala Ala Arg Pro Asn Ser Gly Asn Thr Pro Leu Val Phe); and a beta chain comprising the CDR sequences shown in SEQ ID NO: 11 (Ser Gln Val Thr Met), SEQ ID NO: 12 (Ala Asn Gln Gly Ser Glu Ala), and SEQ ID NO: 13 (Cys Ser Val Glu Gln Asp Thr Asn Thr Gly Glu Leu Phe Phe) as the TCR T1405 described in International Patent Application WO 2014/118236,
an alpha chain comprising the CDR sequences shown in SEQ ID NO: 14 (Asn Ser Ala Phe Gln Tyr), SEQ ID NO: 15 (Thr Tyr Ser Ser Gly Asn), and SEQ ID NO: 16 (Cys Ala Met Ser Asp Thr Gly Asn Gln Phe Tyr Phe); and a beta chain comprising the CDR sequences shown in SEQ ID NO: 17 (Pro Arg His Asp Thr), SEQ ID NO: 18 (Phe Tyr Glu Lys Met Gln), and SEQ ID NO: 19 (Cys Ala Ser Ser Phe Arg Gly Gly Gly Ala Asn Val Leu Thr Phe) as the TCR T1705 described in International Patent Application WO 2014/118236,
an alpha chain comprising the CDR sequences shown in SEQ ID NO: 20 (Thr Ile Ser Gly Thr Asp Tyr), SEQ ID NO: 21 (Gly) and SEQ ID NO: 22 (Cys Ile Leu Phe Asn Phe Asn Lys Phe Tyr Phe); and a beta chain comprising the CDR sequences shown in SEQ ID NO: 23 (Leu Asn His Asn Val), SEQ ID NO: 24 (Tyr Tyr Asp Lys Asp Phe), and SEQ ID NO: 25 (Cys Ala Thr Ser Ser Gly Glu Thr Asn Glu Lys Leu Phe Phe), as the TCR R37P1C9 described in International Patent Application WO 2018/104438,
   an alpha chain comprising the CDR sequences shown in SEQ ID NO: 26 (Asp Ser Ala Ser Asn Tyr), SEQ ID NO: 27 (Ile Arg Ser) and SEQ ID NO: 28 (Cys Ala Ala Ser Pro Thr Gly Gly Tyr Asn Lys Leu Ile Phe); and a beta chain comprising the CDR sequences shown in SEQ ID NO: 29 (Met Asn His Glu Tyr), SEQ ID NO: 30 (Ser Val Gly Ala Gly Ile), and SEQ ID NO: 31 (Cys Ala Ser Ser Leu Gly Gly Ala Ser Gln Glu Gln Tyr Phe) as the TCR R35P3A4 described in International Patent Application WO 2018/104438; or
an alpha chain comprising the CDR sequences shown in SEQ ID NO: 32 (Thr Ser Glu Ser Asn Tyr Tyr), SEQ ID NO: 33 (Gln Glu Ala Tyr) and SEQ ID NO: 34 (Cys Ala Phe Gly Tyr Ser Gly Gly Gly Ala Asp Gly Leu Thr Phe); and a beta chain comprising the CDR sequences shown in SEQ ID NO: 35 (Ser Gly His Asp Thr), SEQ ID NO: 36 (Tyr Tyr Glu Glu Glu Glu), and SEQ ID NO: 37 (Cys Ala Ser Ser Asn Glu Gly Gln Gly Trp Glu Ala Glu Ala Phe Phe) as the TCR R37P1H1 described in International Patent Application WO 2018/104438.

In accordance with the above, in illustrative examples the TCR used herein may have the sequence of the alpha chain or the beta chain of any of the TCRs T1367, T1405, T1705 (all of which are described in International Patent Application WO 2014/118236) or of any the TCRs R37P1C9, R26P2A6, R26P3H1, R42P3A9, R43P3F2, R43P3G5 or R59P2E (all of which are described in International Patent Application WO 2018/104438 and the corresponding U.S. Pat. No. 10,874,731), the TCR T15.8-4.3-83 (that is described in International Patent Application WO20/2020131), the TCR “MA2” (that is described in International Patent Application WO2018/170338) or the avidity optimized TCR hT2 described in Bassan et al, “Avidity optimization of a MAGE-A1-specific TCR with somatic hypermutation”, Eur. J. Immunol. 2021. 51: 1505-1518. In further illustrative examples, a TCR used herein may have the sequence of the CDRs of both the alpha chain and the beta chain of any of the TCRs T1367, T1405, T1705, R37P1C9, R26P2A6, R26P3H1, R42P3A9, R43P3F2, R43P3G5 or R59P2E. In further illustrative examples, a TCR used herein may have the sequence of both the alpha chain and the beta chain of any of the TCRs T1367, T1405, T1705, R37P1C9, R26P2A6, R26P3H1, R42P3A9, R43P3F2, R43P3G5 or R59P2E.

When cellular product/agents such as genetically modified T cells or natural killer (NK) cells are used for the treatment of solid tumors as described herein, such cells can be used in any suitable dosage (therapeutically effective amount). The dosage of the T cells or NK cells administered to the patient, defined as the total number of T cells, may be from about 0.5×10⁷ T cells to about 1×10¹⁰ T cells. Exemplary dosages of the T cells or NK cells administered to the patient, defined as the total number of T cells or NK cells, may be about 0.75×1×10⁸ T cells or NK cells, about 1×10⁸ cells T cells or NK cells, about 1×10⁹ T cells or NK cells, about 3×10⁹ T cells or NK cells, about 4×10⁹ T cells or NK cells, about 5×10⁹ T cells or NK cells, about 6×10⁹ T cells or NK cells, about 7×10⁹ T cells or NK cells, about 8×10⁹ T cells or NK cells or about 9×10⁹T cells or NK cells. The dosage of the T cells or NK cells administered to the patient, defined as the total number of T cells or NK cells, may thus be in the range of about 1×10⁹ cells to about 9×10⁹ cells or in the range of about 3×10⁹ cells to about 9×10⁹ cells. It is noted here that as used herein with respect to the dosage/number of cells used for administration, the term “about” means to include a deviation from the respective value of up to 1%, of up to 2%, of up to 3%, of up to 4%, of up to 5%, or up to and including 10% of the given value. This means, for example, a dosage of “about 1×10⁹ T cells” may include a total number of cells ranging form 1×10⁹%±10%, i.e. from 0.9×10⁹ to 1.1×10⁹ of T cells expressing a MAGE-A1 binding TCR. In other illustrative embodiments, the dosage of the T cells or NK cells administered to the patient, defined as the total number of T cells, may be from 0.5×10⁷ T cells to 1×10¹⁰ T cells.

In line with the commonly used treatments with such cellular products, the T cells or NK cells may be administered as a single dose by any suitable way of administration. Typically, an adoptive cell therapy agent such as genetically modified T cells or NK cells are administered by infusion, for example, intravenous infusion. The infusion may be carried out over any suitable period of time, for example, with a period of as short as only about 15 minutes, about 30 minutes, about 45 minutes or a period of time of up to one hour.

In case genetically modified T cells are used herein, the patient to be treated may be selected according to the HLA genotype to make sure that the patient expresses the HLA molecule which presents the MAGE-A1 epitope to which the TCR binds. Accordingly, the HLA-genotype of the patient may be determined herein. Since known TCRs such as the TCRs T1367, R26P2A6 or T15.8-4.3-83 are known to be HLA-A2 restricted, in illustrative examples, it is checked for patient selection whether the patient in question has a HLA-A*02 genotype, for example, HLA-A*02:01, HLA-A*02:04, HLA-A*02: 16 or HLA-A*02.

Instead of or in addition to an adoptive cell therapy agent (cellular product) such as genetically modified T or NK cells, a drug-ligand conjugate can also be used as the therapeutically active agent specifically binding MAGE-A1. In such a ligand-drug conjugate, the ligand may be an antibody molecule that specifically binds MAGE-A1. Such therapeutically effective conjugates several of which are already approved for cancer treatment and also known under the term “antibody-drug-conjugates (ADC)” are well known to the person skilled in the art (see in this respect, the review of Drago et al., “Unlocking the potential of antibody-drug conjugates for cancer therapy” Nature Reviews Clinical Oncology volume 18, pages 327-344 (2021)) and can be generated using MAGE-A1 binding antibodies, including the MAGE-1A binding antibody MA454 described herein. Alternatively, or a proteinaceous binding molecule with antibody-like binding properties that specifically binds MAGE-A1. In this context, it is noted that all types of proteinaceous binding molecules with antibody-like binding properties that are described herein for being used as reagent for immunostaining can also be used in preparing drug-ligand conjugates suitable for treatment of solid MAGE-1A positive tumors described herein. The drug used in these ligand-drug conjugates may be any suitable cytotoxic or cytostatic molecule. Examples of cytoxic or cystotatic molecules include but are not limited to maytansinoids, calicheamycins, duocarmycins, tubulysins, amatoxins, dolastatins and auristatins such as monomethyl auristatin E (MMAE) or monomethyl auristatin F (MMAF), pyrrolobenzodiazepine dimers, indolino-benzodiazepine dimers, radioisotopes, therapeutic proteins and peptides (or fragments thereof), kinase inhibitors, MEK inhibitors, KSP inhibitors and prodrugs thereof, to mentioned only a few.

Pharmaceutical Composition

In accordance with the above disclosure, the invention also provides a pharmaceutical composition comprising T cells that express a T cell receptor as described here. In illustrative examples, the pharmaceutical composition comprises T cells expressing a T cell receptor that specifically binds MAGE A1, wherein the T-cell receptor comprises
an alpha chain comprising the CDR sequences shown in SEQ ID NO: 2 (Ile Phe Ser Asn Met Asp Met), SEQ ID NO: 3 (Ile Phe Ser Asn Met Asp Met) and SEQ ID NO: 4 (Cys Ala Glu Ser Ile Gly Ser Asn Ser Gly Tyr Ala Leu Asn Phe); and a beta chain comprising the CDR sequences shown in SEQ ID NO: 5 (Met Asp His Glu Asn), SEQ ID NO: 6 (Ser Tyr Asp Val Lys Met), and SEQ ID NO: 7 (Cys Ala Ser Arg Gly Leu Ala Gly Tyr Glu Gln Tyr Phe);
an alpha chain comprising the CDR sequences shown in SEQ ID NO: 8 (Asp Ser Ala Ser Asn Tyr), SEQ ID NO: 9 (Ile Arg Ser Asn Val Gly Glu), and SEQ ID NO: 10 (Cys Ala Ala Arg Pro Asn Ser Gly Asn Thr Pro Leu Val Phe); and a beta chain comprising the CDR sequences shown in SEQ ID NO: 11 (Ser Gln Val Thr Met), SEQ ID NO: 12 (Ala Asn Gln Gly Ser Glu Ala), and 13 (Cys Ser Val Glu Gln Asp Thr Asn Thr Gly Glu Leu Phe Phe); or
an alpha chain comprising the CDR sequences shown in SEQ ID NO: 14 (Asn Ser Ala Phe Gln Tyr), SEQ ID NO: 15 (Thr Tyr Ser Ser Gly Asn), and SEQ ID NO: 16 (Cys Ala Met Ser Asp Thr Gly Asn Gln Phe Tyr Phe); and a beta chain comprising the CDR sequences shown in SEQ ID NO: 17 (Pro Arg His Asp Thr), SEQ ID NO: 18 (Phe Tyr Glu Lys Met Gln), and SEQ ID NO: 19 (Cys Ala Ser Ser Phe Arg Gly Gly Gly Ala Asn Val Leu Thr Phe);
   and wherein the total number of T cells comprised in the composition is from about 0.5×10⁷T cells to about 1×10¹⁰ T cells. In embodiments of the composition, the total number of T cells comprised in the composition is from 0.5×10⁷ T cells to 1×10¹⁰ T cells.

In illustrative examples of the pharmaceutical composition, the total number of T cells comprised in the composition may be about 0.75×1×10⁸ T cells, about 1×10⁸ cells, about 1×10⁹ T cells, about 3×10⁹ T cells, about 4×10⁹ T cells, about 5×10⁹ T cells, about 6×10⁹ T cells, about 7×10⁹T cells, about 8×10⁹T cells or about 9×10⁹T cells. In other illustrative embodiments the total number of T cells comprised in the composition may be 0.75×1×10⁸ T cells, 1×10⁸ cells, 1×10⁹ T cells, 3×10⁹ T cells, 4×10⁹ T cells, 5×10⁹ T cells, 6×10⁹ T cells, 7×10⁹ T cells, 8×10⁹ T cells or 9×10⁹T cells.

The pharmaceutical composition may further comprise one or more pharmaceutically acceptable carriers. Any pharmaceutically acceptable carrier can be used, as long as the carrier does not impact the viability of the T cells to be administered is suitable for the chosen route of administration of the pharmaceutical composition. The pharmaceutical acceptable carrier may be a physiological saline solution, optionally with components such as human serum albumin that can improve the viability of the T cells that express the MAGE-1A binding TCR. It is also possible that the MAGE-1 A expressing T cells are stored, after their manufacture, in frozen form, for example at a temperature of between −20° C. and −80° C. In this case, the pharmaceutical composition may contain cryo-protectants that have been added to protect the cells from being damaged by the freezing process. Examples of cryoprotectants that may be used here for the freezing of the pharmaceutical composition containing transduced T cells include glycerol, DMSO. These cryoprotectant can be used together with crystalloid solutions such as commercially available HypoThermosol® or PlasmaLyte-A solution which are both approved for infusion and are available in pharmaceutical grade. Other possible media that can be used as carrier in the pharmaceutical composition are media of the “CryoStor family”, commercially available animal protein-free defined cryopreservation media from Biolife Solutions such as CyroStor2 (CS2, an optimized freeze media pre-formulated with 2% DMSO), CyroStor5 (CS5, an optimized freeze media pre-formulated with 5% DMSO), or CyroStor10 (CS10, an optimized freeze media pre-formulated with 10% DMSO).

Assays & Kits

Turning now again to the immunostaining as described here, the present invention als provides the use of the monoclonal IgG1 mouse antibody MA454 or an antigen binding fragment of the antibody MA454 for selecting a patient for treatment of a solid tumor, wherein cells of the tumor express the human melanoma associated antigen 1 (MAGE-A1) (UniProtKB accession number P43355 (MAGA1_HUMAN). In addition, the invention provides the use of the monoclonal IgG1 mouse antibody MA454 or a fragment of the antibody MA454 for predicting whether a patient being diagnosed with a solid tumor will be responsive to treatment of this tumor, wherein cells of the tumor express human melanoma associated antigen 1 (MAGE-A1) (UniProtKB accession number P43355 (MAGA1_HUMAN). In line with the above disclosure, these uses may comprise determining in a tumor cell sample obtained from the patient the fraction of cells that express MAGE-A1, wherein a patient is selected for treatment if a fraction of at least 30% of the cells of the tumor sample are found to express MAGE-A1.

Dealing now with the diagnostic kit of the invention, such a kit is a diagnostic immunostaining kit for selecting a patient for treatment of a solid tumor, wherein cells of the tumor express the human melanoma associated antigen 1 (MAGE-A1) (UniProtKB accession number P43355 (MAGA1_HUMAN), wherein the kit comprises
the monoclonal IgG1 mouse antibody MA454 or a fragment of the antibody MA454 as a primary antibody, and
a secondary antibody capable of binding to the monoclonal IgG1 mouse antibody MA454. Such a kit can be used as companion diagnostic for selecting a patient for treatment of a MAGE-1A positive solid tumor as well as for predicting or monitoring whether a patient being diagnosed with a MAGE-A1 positive solid tumor will be responsive to treatment of this tumor.

As explained above, the kit may further comprise a secondary antibody capable of binding to the primary antibody, the antibody MA454. As explained herein, a secondary binding reagent such as a secondary antibody that comprises (usually by conjugation) typically binds to structurally conserved regions of the primary binding reagent, here the constant domains of the mouse IgG1 antibody, to thereby increase the signal strength of the desired staining.

Accordingly, the secondary antibody used herein may comprise an optically detectable label. The optically detectable label may be a fluorescent label, a chromophore or chromogenic label, an isotope label, or a metal label. In one illustrative example, the optically detectable label is an enzyme catalyzing a chromogenic reaction. Examples of suitable enzymes include, but are not limited to, peroxidase or alkaline phosphatase. These enzymes may be conjugated to a polymer such as a dextran polymer. Suitably labelled secondary antibodies are commercially available, for example, in form of the EnVision Polymer-HRP (Dako catalogue number K4001). This secondary antibody contained in the Envision product is intended for use with primary antibodies from mouse supplied by the user for the qualitative identification of antigens by light microscopy in normal and pathological paraffin-embedded tissues, cryostat tissues or cell preparations. According to the manufacturer, the tissue to be stained can be processed in a variety of fixatives including ethanol, B-5, Bouin's, zinc formalin, and neutral buffered formalin. These secondary antibodies are conjugated with a horse radish peroxidase (HRP) labelled dextran polymer. In examples of the staining kits of the invention, the monoclonal IgG1 mouse antibody MA454 or a fragment of the antibody MA454 as the primary antibody and the secondary antibody are packaged in individual containers and provided to the user together with instructions to use.

In case the tissue staining as described herein using the antibody MA454 or a antigen binding fragment thereof is used as a companion diagnostic, it is possible to carry out the tissue staining on an automated platform. Examples of such automated platforms that are suitable to be used here include the various staining devices offered by Leica Biosystems such as the Leica ST4020 Small Linear Stainer, the HistoCore SPECTRA ST Stainer (a high throughput routine histology stainer) or the fully automated instruments Leica ST5010-CV5030 Integrated Workstation, the BenchMark IHC/ISH slide staining system of Roche Tissue Diagnostics that is being used for fully-automated immunohistochemistry and in situ hybridization slide staining, Artisan Link Pro Special Staining System or the Artisan Link Pro Special Staining System of Agilent/Dako (an in vitro diagnostic device intended to automate slide-based special stains on formalin-fixed, paraffin-embedded tissue sections). Implementing the staining assay as described herein using the antibody MA454, and a secondary antibody (the latter, for example, binding to the constant domains of a mouse IgG1 antibody, as described herein) on such an automated system for use an companion diagnostic is within the ability of the person of average skill in the art.

The invention will be further illustrated by the following non-limiting Experimental Examples.

Sequences as used herein are depicted in below Table 1.

TABLE 1
Sequences as used herein.

| SEQ | | |
|---|---|---|
| ID | | |
| NO. | Name | Sequence |
| 1 | MAGE-A1, | 10 20 30 40 50 |
| | UniProtKB | MSLEQRSLHC KPEEALEAQQ EALGLVCVQA ATSSSSPLVL GTLEEVPTAG |
| | accession | 60 70 80 90 100 |
| | number | STDPPQSPQG ASAFPTTINF TRQRQPSEGS SSREEEGPST SCILESLFRA |
| | P43355 | 110 120 130 140 150 |
| | (MAGA1_HUMAN | VITKKVADLV GFLLLKYRAR EPVTKAEMLE SVIKNYKHCF PEIFGKASES |
| | | 160 170 180 190 200 |
| | | LQLVFGIDVK EADPTGHSYV LVTCLGLSYD GLLGDNQIMP KTGFLIIVLV |
| | | 210 220 230 240 250 |
| | | MIAMEGGHAP EEEIWEELSV MEVYDGREHS AYGEPRKLLT QDLVQEKYLE |
| | | 260 270 280 290 300 |
| | | YRQVPDSDPA RYEFLWGPRA LAETSYVKVL EYVIKVSARV RFFFPSLREA |
| | | ALREEEEGV |
| 2 | alpha chain | Asp Ser Ser Ser Thr Tyr |
| | CDR 1 of TCR | |
| | T1367 | |
| 3 | alpha chain | Ile Phe Ser Asn Met Asp Met |
| | CDR 2 of TCR | |
| | T1367 | |
| 4 | alpha chain | Cys Ala Glu Ser Ile Gly Ser Asn Ser Gly Tyr Ala Leu Asn Phe |
| | CDR 3 of TCR | |
| | T1367 | |
| 5 | beta chain | Met Asp His Glu Asn |
| | CDR 1 of TCR | |
| | T1367 | |
| 6 | beta chain | Ser Tyr Asp Val Lys Met |
| | CDR 2 of TCR | |
| | T1367 | |
| 7 | beta chain | Cys Ala Ser Arg Gly Leu Ala Gly Tyr Glu Gln Tyr Phe |
| | CDR 3 of TCR | |
| | T1367 | |
| 8 | alpha chain | Asp Ser Ala Ser Asn Tyr |
| | CDR 1 of TCR | |
| | T1405 | |
| 9 | alpha chain | Ile Arg Ser Asn Val Gly Glu |
| | CDR 2 of TCR | |
| | T1405 | |
| 10 | alpha chain | Cys Ala Ala Arg Pro Asn Ser Gly Asn Thr Pro Leu Val Phe |
| | CDR 3 of TCR | |
| | T1405 | |
| 11 | beta chain | Ser Gln Val Thr Met |
| | CDR 1 of TCR | |
| | T1405 | |
| 12 | beta chain | Ala Asn Gln Gly Ser Glu Ala |
| | CDR 2 of TCR | |
| | T1405 | |
| 13 | beta chain | Cys Ser Val Glu GIn Asp Thr Asn Thr Gly Glu Leu Phe Phe |
| | CDR 3 of TCR | |
| | T1405 | |
| 14 | alpha chain | Asn Ser Ala Phe Gln Tyr |
| | CDR 1 of TCR | |
| | T1705 | |
| 15 | alpha chain | Thr Tyr Ser Ser Gly Asn |
| | CDR 2 of TCR | |
| | T1705 | |
| 16 | alpha chain | Cys Ala Met Ser Asp Thr Gly Asn Gln Phe Tyr Phe |
| | CDR 2 of TCR | |
| | T1705 | |
| 17 | beta chain | Pro Arg His Asp Thr |
| | CDR 1 of TCR | |
| | T1705 | |
| 18 | beta chain | Phe Tyr Glu Lys Met Gln |
| | CDR 2 of TCR | |
| | T1705 | |
| 19 | beta chain | Cys Ala Ser Ser Phe Arg Gly Gly Gly Ala Asn Val Leu Thr Phe |
| | CDR 3 of TCR | |
| | T1705 | |
| 20 | alpha chain | Thr Ile Ser Gly Thr Asp Tyr |
| | CDR 1 of TCR | |
| | R37P1C9 | |
| 21 | alpha chain | Gly |
| | CDR 2 of TCR | |
| | R37P1C9 | |
| 22 | alpha chain | Cys Ile Leu Phe Asn Phe Asn Lys Phe Tyr Phe |
| | CDR 3 of TCR | |
| | R37P1C9 | |
| 23 | beta chain | Leu Asn His Asn Val |
| | CDR 1 of TCR | |
| | R37P1C9 | |
| 24 | beta chain | Tyr Tyr Asp Lys Asp Phe |
| | CDR 2 of TCR | |
| | R37P1C9 | |
| 25 | beta chain | Cys Ala Thr Ser Ser Gly Glu Thr Asn Glu Lys Leu Phe Phe |
| | CDR 3 of TCR | |
| | R37P1C9 | |
| 26 | alpha chain | Asp Ser Ala Ser Asn Tyr |
| | CDR 1 of TCR | |
| | R35P3A4 | |
| 27 | alpha chain | Ile Arg Ser |
| | CDR 2 of TCR | |
| | R35P3A4 | |
| 28 | alpha chain | Cys Ala Ala Ser Pro Thr Gly Gly Tyr Asn Lys Leu Ile Phe |
| | CDR 3 of TCR | |
| | R35P3A4 | |
| 29 | beta chain | Met Asn His Glu Tyr |
| | CDR 1 of TCR | |
| | R35P3A4 | |
| 30 | beta chain | Ser Val Gly Ala Gly Ile |
| | CDR 2 of TCR | |
| | R35P3A4 | |
| 31 | beta chain | Cys Ala Ser Ser Leu Gly Gly Ala Ser Gln Glu GIn Tyr Phe |
| | CDR 3 of TCR | |
| | R35P3A4 | |
| 32 | alpha chain | Thr Ser Glu Ser Asn Tyr Tyr |
| | CDR 1 of TCR | |
| | R37P1H1 | |
| 33 | alpha chain | Gln Glu Ala Tyr |
| | CDR 2 of TCR | |
| | R37P1H1 | |
| 34 | alpha chain | Cys Ala Phe Gly Tyr Ser Gly Gly Gly Ala Asp Gly Leu Thr Phe |
| | CDR 3 of TCR | |
| | R37P1H1 | |
| 35 | beta chain | Ser Gly His Asp Thr |
| | CDR 1 of TCR | |
| | R37P1H1 | |
| 36 | beta chain | Tyr Tyr Glu Glu Glu Glu |
| | CDR 2 of TCR | |
| | R37P1H1 | |
| 37 | beta chain | Cys Ala Ser Ser Asn Glu Gly Gln Gly Trp Glu Ala Glu Ala Phe Phe |
| | CDR 3 of TCR | |
| | R37P1H1 | |

### EXPERIMENTAL EXAMPLES

### Example 1. Immunohistochemical Analysis of MAGE-A1 Expression in Human Cancers and Normal Tissues

Melanoma Antigen Gene A1 (MAGE-A1) is a member of the cancer testis antigen family. This analysis aimed at 1) the validation of the antibody MA454) for analysis of MAGE-A1 expression by means of immunohistochemistry, 2) MAGE-A1 immunohistochemical expression analysis of human normal and cancer tissues and 3) estimation of the heterogeneity of MAGE-A1 expression in selected tumor types and in advanced/metastatic disease vs. primary disease.

### 1.1 Materials and Methods

### Antibody

Anti-MAGE-A1 clone MA454, mouse monoclonal IgG1 (200 μg/ml), cat. #ab193330 (Abcam, Cambridge, UK) was used in this study.

### Immunohistochemistry (IHC) Protocol:

Slide Preparation
deparaffinize TMAs overnight in Xylene
rehydrate in descending ethanol series (100%, 90%, 80%, 70%)
rinse 5 min in TBS buffer

Pretreatment (Epitope Retrieval)
incubate in autoclave at 121° C. for 5 min in pH 7.8 EDTA buffer
wash 5 min in TBS buffer

Peroxidase Blocking
incubate 10 min in 3% H₂O₂
rinse 2×5 min in TBS buffer

Primary Antibody and Visualization
apply 100-200 μl pre-diluted primary antibody (dilution 1:25 of MA454, 8 μg/ml, for maximal sensitivity of the tissue analysis or dilution 1:150 for the cell line TMA) to each slide
incubate for 2 h at room temperature in moist chamber
thoroughly remove Ab solution with TBS buffer
again, rinse 2×5 min in TBS buffer

Detection
incubate slide with EnVision Polymer-HRP (Dako K4001) according to the manufacturer's instructions
rinse 2×5 min in TBS buffer

Chromogen
cover slides for 10 min with DAB-Chromogen (Liquid DAB DAKO Code No.: K 3467) according to the manufacturer's instructions
wash slides in distilled water
counterstain for 20 sec with hematoxylin (Harris Hämatoxylin HTX 31000, Medite GmbH)
rinse with tap water
rinse with HCl-Ethanol (1% conc. HCl in ethanol) to remove excess hematoxylin
rinse for 5 min in tap water
dehydrate in ascending ethanol series (70%, 80%, 90%, 100%) and xylene
apply mounting medium and coverslip

Tissues

This study involved 9 different tissue microarrays (TMAs):

The MAGE-A1 cross-reactivity test TMA was constructed from formalin-fixed, paraffin embedded 293T cells transfected with expression cassettes for MAGE-A1 and other members of the MAGE-A protein family (Table 1). It was designed to test for possible cross-reactivity of MA454 with other members of the MAGE-A protein family. Each cell line was represented by duplicate 0.6 mm punches. In addition, human testis tissue was added as positive tissue controls.

TABLE 1
Layout of the MAGE-A1 cross reactivity test TMA. 293T-wt = wildtype
untransfected 293T cells. MAGE A1-12 indicates the particular transfected
MAGE-A expression vector.

| | | Expected MAGE- |
|---|---|---|
| Array coordinate | Cells | A1 status |
| A 1a | 293T-MAGE A1 | Positive |
| A 1b | 293T-MAGE A1 | Positive |
| A 2a | 293T-wt | negative |
| A 2b | 293T-wt | negative |
| A 3a | 293T-MAGE A2 | negative |
| A 3b | 293T-MAGE A2 | negative |
| A 3c | 293T-MAGE A3 | negative |
| A 3d | 293T-MAGE A3 | negative |
| A 3e | 293T-MAGE A4 | negative |
| A 3f | 293T-MAGE A4 | negative |
| A 3g | 293T-MAGE A5 | negative |
| A 3h | 293T-MAGE A5 | negative |
| A 3i | 293T-MAGE A6 | negative |
| A 3k | 293T-MAGE A6 | negative |
| A 4a | 293T-MAGE A8 | negative |
| A 4b | 293T-MAGE A8 | negative |
| A 4c | 293T-MAGE A9 | negative |
| A 4d | 293T-MAGE A9 | negative |
| A 4e | 293T-MAGE A10 | negative |
| A 4f | 293T-MAGE A10 | negative |
| A 4g | 293T-MAGE A11 | negative |
| A 4h | 293T-MAGE A11 | negative |
| A 4i | 293T-MAGE A12 | negative |
| A 4k | 293T-MAGE A12 | negative |
| A 5a | human testis | positive |
| A 5b | human testis | positive |
| A 5c | human testis | positive |
| A 5d | human testis | positive |

The Multi-tumor TMA (MTA 5) is composed of 3,449 tissue samples (0.6 mm punches, one punch per tumor) from 83 human cancer types distributed across seven TMA blocks termed MTA 5.2 A-G. Formalin fixed (buffered neutral aqueous four percent solution), paraffin embedded tumor material was utilized. No clinico-pathological data other than the histological tumor type is available. The detailed composition of this TMA is given in the results section.

The Normal tissue TMA (NTA 9) includes 8 samples each (from 8 different donors) of 76 healthy human organ systems (total 608 samples). Formalin fixed (buffered neutral aqueous four percent solution), paraffin embedded material was utilized. The detailed composition of the TMA is given in the appendix of this report.

The Bladder Cancer Heterogeneity TMA (BLA 1.2) includes 5 samples (0.6 mm punches) each from 105 muscle invasive bladder cancers. Formalin fixed (buffered neutral aqueous four percent solution), paraffin embedded tumor material was utilized. No further histopathological or clinical data is available.

The Breast Cancer Prognosis TMA (BRE 1.1) includes one 0.6 mm punch each from 849 breast cancers. Formalin fixed (buffered neutral aqueous four percent solution), paraffin embedded tumor material was utilized. Histopathological (histological subtype, tumor stage, nodal stage, distant metastasis stage, histological grade), molecular (estrogen receptor, HER2) and clinical data (patient survival) is available.

The Esophageal Cancer Prognosis TMA (ESO 1.1) includes one 0.6 mm punch each from 299 primary esophageal cancers and additional 0.6 mm punches (each one punch per metastasis) from matched 154 lymph node and/or distant metastases. Formalin fixed (buffered neutral aqueous four percent solution), paraffin embedded tumor material was utilized. Histopathological (histological subtype, tumor stage, nodal stage, distant metastasis stage, histological grade and resection margin status) and clinical data (patient survival) is available.

The Lung Cancer Prognosis TMA (LUN 1.1) includes one 0.6 mm punch each from 616 primary lung cancers. Formalin fixed (buffered neutral aqueous four percent solution), paraffin embedded tumor material was utilized. Histopathological (histological subtype, tumor stage, nodal stage, distant metastasis stage, histological grade and UICC) and clinical data (patient survival) is available.

The Lung Cancer Heterogeneity TMA (LUN 4.2) includes 8 samples (0.6 mm punches) each from 146 primary lung adenocarcinomas and up to 4 samples from lymph node metastases matched to 78 of the 146 primary cancers. Formalin fixed (buffered neutral aqueous four percent solution), paraffin embedded tumor material was utilized. No further histopathological or clinical data is available.

The Stomach Cancer Heterogeneity TMA (STO 2.2) includes 9 samples (0.6 mm punches) each from 113 primary stomach cancers and 3 samples each from 1-3 lymph node metastases matched to 61 of the 113 primary cancers. Formalin fixed (buffered neutral aqueous four percent solution), paraffin embedded tumor material was utilized. No further histopathological or clinical data is available.

### 2. Immunohistochemistry Analysis

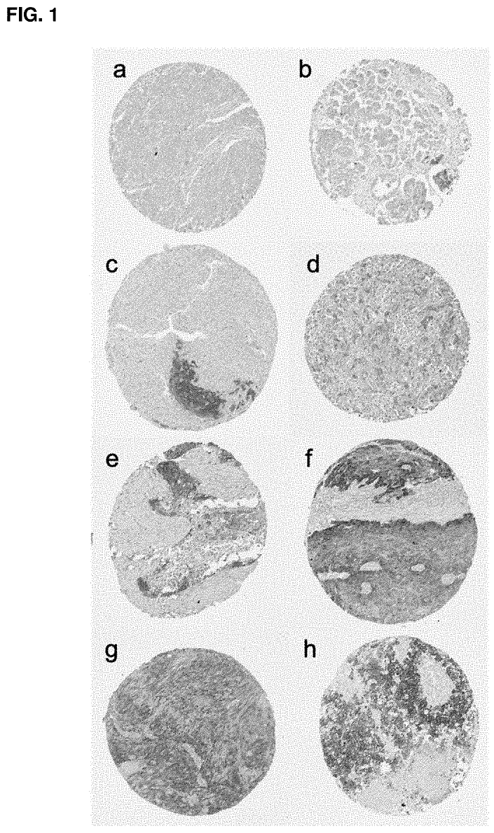

The immunohistochemistry experiments were evaluated as follows. For each tumor tissue spot, the staining intensity was recorded in a four-step scale (0, 1+, 2+, 3+) and the fraction of stained tumor cells was estimated. A tumor was considered positive if at least 5% of the tumor cells showed immunostaining of intensity 1+ or more). Tumor spots were then categorized according to the fraction of tumor cells with MAGE-A1 positive staining into 4 groups: negative (no staining), <80% pos. tumor cells, ≥80 (but <100%) positive tumor cells, and 100% positive tumor cells. Examples of the different scores are shown in FIGS. 1a to 1h. It is noted here that FIG. 1h shows a tumor cell sample that contains 100% MAGE-1A positive cells contains lots of normal surrounding tissue and non-tumoral interstitium. For this reason, the staining of the tissue sample in FIG. 1h shows more “uncoloured sections”, i.e. tumor cells that are not MAGE-A1 positive than the tumor cell sample shown in FIG. 1g which is determined to have 90% MAGE-A1 positive cells and appears to be essentially fully stained tissue sample.

### 3. Results

### 3.1 Specificity of MAGE-A1 Binding Antibody MA454

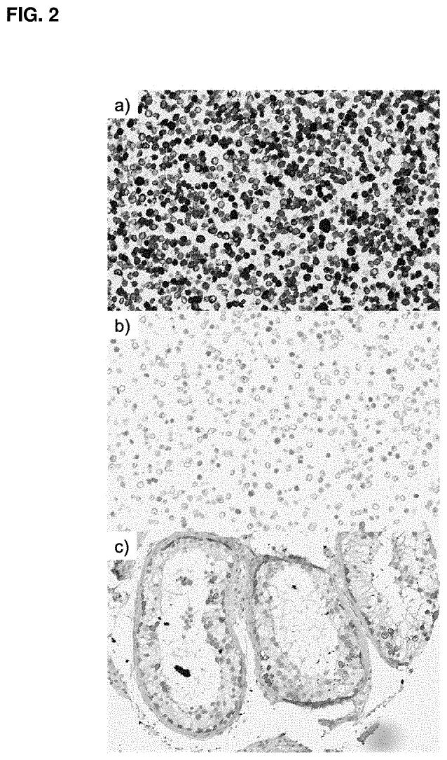

In order to exclude possible cross reactivity of MA454 with other members of the MAGE-A protein family, the “MAGE-A1 cross-reactivity test TMA” was analyzed. MA454 stained 293T cells transfected with MAGE-A1, but not non-transfected 293T cells or any other cell line transfected with other MAGE-A proteins. Human testis control tissue stained positive as expected. Representative images are depicted in FIG. 2. In more detail. FIG. 2a shows 293T-MAGE A1 cells with positive staining. The different staining intensities in individual cells result from variable amounts of MAGE-A1 expression. FIG. 2b shows as a negative control untransfected 293T cells. FIG. 2c shows as a positive control human testis tissue.

### 3.2 MAGE-A1 Staining in Normal Tissues

Eight independent samples were analyzed per tissue type. Detectable MAGE-A1 staining was strictly limited to testis, with intense staining of spermatogonia and spermatocytes. Normal tissue types without detectable MAGE-A1 staining included.

| | | |
|---|---|---|
| adrenal gland | fat | Prostate |
| anal canal | gallbladder | Rectum |
| Aorta | hair follicles | sebaceous glands |
| Appendix | heart | seminal vesicle |
| bone marrow | ileum | sinus paranasales |
| Breast | kidney | skin |
| Bronchus | lip | spleen |
| Cerebellum | liver | stomach |
| Cerebrum | lung | striated muscle |
| Colon | lymph node | sublingual gland |
| Duodenum | oral cavity | submandibular gland |
| Ectocervix | ovary | thymus |
| Endocervix | pancreas | thyroid gland |
| Endometrium | parathyroid | tongue |
| Epididymis | penis | tonsil |
| Esophagus | pituitary gland | urinary bladder |
| fallopian tube | placenta | uterus |

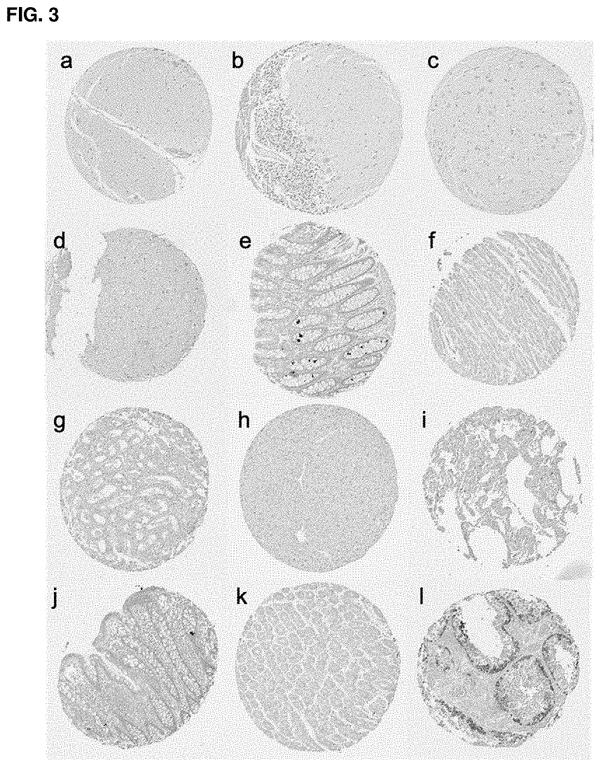

Examples of normal tissues with and without MAGE-A1 staining are shown in FIG. 3 (a) cerebellum cortex, b) cerebellum grey matter, c) cerebrum grey matter, d) cerebrum white matter, e) colon mucosa, f) heart, g) kidney cortex, h) liver, i) lung, j) small intestine, k) stomach corpus, I) testis).

### 3.3. Multitumor Array (MTA 5) Analysis of MAGE-A1 Expression

Immunostaining was analyzable for 2,936 (85%) of the 3,449 arrayed cancer samples. A total of 513 tissue spots did not yield interpretable results either because of missing tissue spots on the TMA slide or because of lack of tumor cells in the tissue spot. Positive MAGE-A1 immunostaining (i.e., at least 5% tumor cells with staining intensity 1+ or more) was found in 204 (6.9%) of cancer samples.

Tumors were categorized for the fraction of positive samples per tumor type as well as for the fraction of positive tumor cells in three groups (<80%, ≥80% and 100%), irrespective of the staining intensity.

Tumor types with most frequent (≥20% of samples) MAGE-A1 positivity (any visible staining) included malignant melanoma (36% positive), squamous cell carcinomas of the lung (27%), esophagus (26%) and vagina (24%), muscle invasive bladder cancer (urothelial carcinoma, 26%), adenocarcinoma of the esophagus (24%), tubular breast cancer (23%) and seminoma (20%). In these tumor types, the fraction of samples with ≥80% positive tumor cells was highly variable and ranged between 10% (n=10 seminomas) and 100% (n=3 tubular breast cancers).

Tumor types which most frequently displayed a rather homogeneous MAGE-A1 expression (i.e., 80-100% of the tumor cells positive) included tubular breast cancer (23% of samples), squamous cell carcinomas of the lung (23%) and esophagus (17%), invasive breast carcinoma of no special type (17%), malignant melanoma (13%), muscle invasive urinary bladder cancer (12%), small cell neuroendocrine carcinoma of the urinary bladder (11%), diffuse gastric adenocarcinoma (11%), and osteosarcoma (10%).

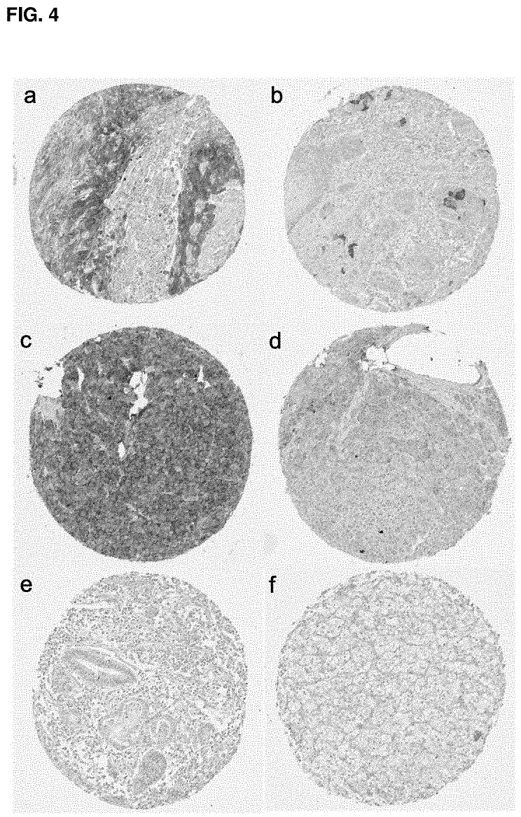

Example images of MAGE-A1 staining in tumor cells are shown in FIG. 4. All data are summarized in Table 2.

TABLE 2
MAGE-A1 staining results of the multitumor array.

| | | | | Samples | | Samples | | Samples | |
|---|---|---|---|---|---|---|---|---|---|
| | | | | with less | | with 80- | | with | |
| | | | | than 80% | | 100% | | 100% | |
| | | Negative | | positive | | positive | | positive | |
| | | samples | | tumor cells | | tumor cells | | tumor cells | |
| Tumors of | Pilomatrixoma | 96.3% | (26/27) | 3.7% | (1/27) | 0.0% | (0/27) | 0.0% | (0/27) |
| the skin | Basal cell | 93.6% | (44/47) | 2.1% | (1/47) | 4.3% | (2/47) | 4.3% | (2/47) |
| | carcinoma | | | | | | | | |
| | Benign nevus | 100.0% | (28/28) | 0.0% | (0/28) | 0.0% | (0/28) | 0.0% | (0/28) |
| | Squamous cell | 93.8% | (45/48) | 6.3% | (3/48) | 0.0% | (0/48) | 0.0% | (0/48) |
| | carcinoma of | | | | | | | | |
| | the skin | | | | | | | | |
| | Malignant | 64.4% | (29/45) | 22.2% | (10/45) | 13.3% | (6/45) | 13.3% | (6/45) |
| | melanoma | | | | | | | | |
| | Merkel cell | 95.6% | (43/45) | 4.4% | (2/45) | 0.0% | (0/45) | 0.0% | (0/45) |
| | carcinoma | | | | | | | | |
| Tumors of | Squamous cell | 83.7% | (41/49) | 10.2% | (5/49) | 6.1% | (3/49) | 2.0% | (1/49) |
| the head and | carcinoma of the | | | | | | | | |
| neck | larynx | | | | | | | | |
| | Oral squamous | 86.0% | (43/50) | 12.0% | (6/50) | 2.0% | (1/50) | 2.0% | (1/50) |
| | cell carcinoma | | | | | | | | |
| | (floor of the mouth) | | | | | | | | |
| | Squamous cell | 72.7% | (16/22) | 4.5% | (1/22) | 22.7% | (5/22) | 9.1% | (2/22) |
| | carcinoma of the | | | | | | | | |
| | lung | | | | | | | | |
| | Large cell | 89.5% | (17/19) | 5.3% | (1/19) | 5.3% | (1/19) | 5.3% | (1/19) |
| | carcinoma of the | | | | | | | | |
| | lung | | | | | | | | |
| | Adenocarcinoma | 90.9% | (20/22) | 4.5% | (1/22) | 4.5% | (1/22) | 0.0% | (0/22) |
| | of the lung | | | | | | | | |
| | Bronchioalveolar | 100.0% | (6/6) | 0.0% | (0/6) | 0.0% | (0/6) | 0.0% | (0/6) |
| | carcinoma of the | | | | | | | | |
| | lung | | | | | | | | |
| | Small cell | 100.0% | (17/17) | 0.0% | (0/17) | 0.0% | (0/17) | 0.0% | (0/17) |
| | carcinoma of the | | | | | | | | |
| | lung | | | | | | | | |
| | Malignant | 93.0% | (40/43) | 0.0% | (0/43) | 7.0% | (3/43) | 4.7% | (2/43) |
| | mesothelioma | | | | | | | | |
| | Pleomorphic | 97.8% | (45/46) | 2.2% | (1/46) | 0.0% | (0/46) | 0.0% | (0/46) |
| | adenoma of the | | | | | | | | |
| | parotid gland | | | | | | | | |
| | Warthin tumor of | 100.0% | (46/46) | 0.0% | (0/46) | 0.0% | (0/46) | 0.0% | (0/46) |
| | the parotid gland | | | | | | | | |
| | Basal cell | 100.0% | (15/15) | 0.0% | (0/15) | 0.0% | (0/15) | 0.0% | (0/15) |
| | adenoma of the | | | | | | | | |
| | salivary gland | | | | | | | | |
| Tumors of | Squamous cell | 75.7% | (28/37) | 18.9% | (7/37) | 5.4% | (2/37) | 0.0% | (0/37) |
| the female | carcinoma of the | | | | | | | | |
| genital tract | vagina | | | | | | | | |
| | Squamous cell | 84.4% | (38/45) | 11.1% | (5/45) | 4.4% | (2/45) | 0.0% | (0/45) |
| | carcinoma of the | | | | | | | | |
| | vulva | | | | | | | | |
| | Squamous cell | 95.8% | (46/48) | 4.2% | (2/48) | 0.0% | (0/48) | 0.0% | (0/48) |
| | carcinoma of the | | | | | | | | |
| | cervix | | | | | | | | |
| | Adenocarcinoma | 97.9% | (46/47) | 0.0% | (0/47) | 2.1% | (1/47) | 0.0% | (0/47) |
| | of the cervix | | | | | | | | |
| | Endometrioid | 95.7% | (45/47) | 2.1% | (1/47) | 2.1% | (1/47) | 2.1% | (1/47) |
| | endometrial | | | | | | | | |
| | carcinoma | | | | | | | | |
| | Endometrial | 97.7% | (42/43) | 0.0% | (0/43) | 2.3% | (1/43) | 0.0% | (0/43) |
| | serous carcinoma | | | | | | | | |
| | Endometrial | 100.0% | (12/12) | 0.0% | (0/12) | 0.0% | (0/12) | 0.0% | (0/12) |
| | stromal sarcoma | | | | | | | | |
| | Carcinosarcoma | 91.3% | (42/46) | 6.5% | (3/46) | 2.2% | (1/46) | 2.2% | (1/46) |
| | Endometroid | 81.3% | (26/32) | 12.5% | (4/32) | 6.3% | (2/32) | 3.1% | (1/32) |
| | carcinoma of the | | | | | | | | |
| | ovary | | | | | | | | |
| | Serous carcinoma | 89.6% | (43/48) | 8.3% | (4/48) | 2.1% | (1/48) | 2.1% | (1/48) |
| | of the ovary | | | | | | | | |
| | Mucinous | 96.0% | (24/25) | 4.0% | (1/25) | 0.0% | (0/25) | 0.0% | (0/25) |
| | carcinoma of the | | | | | | | | |
| | ovary | | | | | | | | |
| | Brenner tumor | 100.0% | (8/8) | 0.0% | (0/8) | 0.0% | (0/8) | 0.0% | (0/8) |
| Tumors of | Invasive breast | 82.6% | (19/23) | 0.0% | (0/23) | 17.4% | (4/23) | 8.7% | (2/23) |
| the breast | carcinoma of no | | | | | | | | |
| | special type | | | | | | | | |
| | Lobular carcinoma | 91.2% | (31/34) | 2.9% | (1/34) | 5.9% | (2/34) | 5.9% | (2/34) |
| | of the breast | | | | | | | | |
| | Medullary | 92.9% | (13/14) | 0.0% | (0/14) | 7.1% | (1/14) | 7.1% | (1/14) |
| | carcinoma of the | | | | | | | | |
| | breast | | | | | | | | |
| | Tubular carcinoma | 76.9% | (10/13) | 0.0% | (0/13) | 23.1% | (3/13) | 23.1% | (3/13) |
| | of the breast | | | | | | | | |
| | Mucinous | 95.2% | (20/21) | 4.8% | (1/21) | 0.0% | (0/21) | 0.0% | (0/21) |
| | carcinoma of the | | | | | | | | |
| | breast | | | | | | | | |
| | Phyllodes tumor | 97.2% | (35/36) | 0.0% | (0/36) | 2.8% | (1/36) | 2.8% | (1/36) |
| | of the breast | | | | | | | | |
| Tumors of | Adenomatous | 100.0% | (46/46) | 0.0% | (0/46) | 0.0% | (0/46) | 0.0% | (0/46) |
| the digestive | polyp, low-grade | | | | | | | | |
| system | dysplasia | | | | | | | | |
| | Adenomatous | 100.0% | (44/44) | 0.0% | (0/44) | 0.0% | (0/44) | 0.0% | (0/44) |
| | polyp, high-grade | | | | | | | | |
| | dysplasia | | | | | | | | |
| | Adenocarcinoma | 100.0% | (20/20) | 0.0% | (0/20) | 0.0% | (0/20) | 0.0% | (0/20) |
| | of the colon | | | | | | | | |
| | Adenocarcinoma | 100.0% | (7/7) | 0.0% | (0/7) | 0.0% | (0/7) | 0.0% | (0/7) |
| | of the small | | | | | | | | |
| | intestine | | | | | | | | |
| | Gastric | 84.2% | (16/19) | 5.3% | (1/19) | 10.5% | (2/19) | 5.3% | (1/19) |
| | adenocarcinoma, | | | | | | | | |
| | diffuse type | | | | | | | | |
| | Gastric | 100.0% | (22/22) | 0.0% | (0/22) | 0.0% | (0/22) | 0.0% | (0/22) |
| | adenocarcinoma, | | | | | | | | |
| | intestinal type | | | | | | | | |
| | Adenocarcinoma | 76.0% | (19/25) | 16.0% | (4/25) | 8.0% | (2/25) | 8.0% | (2/25) |
| | of the esophagus | | | | | | | | |
| | Squamous cell | 73.9% | (17/23) | 8.7% | (2/23) | 17.4% | (4/23) | 13.0% | (3/23) |
| | carcinoma of the | | | | | | | | |
| | esophagus | | | | | | | | |
| | Squamous cell | 88.9% | (32/36) | 2.8% | (1/36) | 8.3% | (3/36) | 5.6% | (2/36) |
| | carcinoma of the | | | | | | | | |
| | anal canal | | | | | | | | |
| | Cholangiocarcinoma | 97.8% | (44/45) | 2.2% | (1/45) | 0.0% | (0/45) | 0.0% | (0/45) |
| | Hepatocellular | 86.0% | (43/50) | 14.0 | (7/50) | 0.0% | (0/50) | 0.0% | (0/50) |
| | carcinoma | | | | | | | | |
| | Ductal | 97.3% | (36/37) | 0.0% | (0/37) | 2.7% | (1/37) | 0.0% | (0/37) |
| | adenocarcinoma of | | | | | | | | |
| | the pancreas | | | | | | | | |
| | Pancreas, | 100.0% | (16/16) | 0.0% | (0/16) | 0.0% | (0/16) | 0.0% | (0/16) |
| | ampullary | | | | | | | | |
| | adenocarcinoma | | | | | | | | |
| | Pancreas, | 81.4% | (35/43) | 11.6% | (5/43) | 7.0% | (3/43) | 7.0% | (3/43) |
| | neuroendocrine | | | | | | | | |
| | carcinoma (NEC) | | | | | | | | |
| | Gastrointestinal | 91.1% | (41/45) | 6.7% | (3/45) | 2.2% | (1/45) | 2.2% | (1/45) |
| | stroma tumor | | | | | | | | |
| | (GIST) | | | | | | | | |
| Tumors of | Non-invasive | 100.0% | (45/45) | 0.0% | (0/45) | 0.0% | (0/45) | 0.0% | (0/45) |
| the urinary | papillary urothelial | | | | | | | | |
| system | carcinoma, pTa | | | | | | | | |
| | Urothelial | 74.0% | (37/50) | 14.0% | (7/50) | 12.0% | (6/50) | 2.0% | (1/50) |
| | carcinoma, pT2-4 | | | | | | | | |
| | Small cell | 83.3% | (15/18) | 5.6% | (1/18) | 11.1% | (2/18) | 5.6% | (1/18) |
| | neuroendocrine | | | | | | | | |
| | carcinoma of the | | | | | | | | |
| | bladder | | | | | | | | |
| | Clear cell renal cell | 100.0% | (47/47) | 0.0% | (0/47) | 0.0% | (0/47) | 0.0% | (0/47) |
| | carcinoma | | | | | | | | |
| | Papillary renal cell | 91.5% | (43/47) | 8.5% | (4/47) | 0.0% | (0/47) | 0.0% | (0/47) |
| | carcinoma | | | | | | | | |
| | Chromophobe | 100.0% | (48/48) | 0.0% | (0/48) | 0.0% | (0/48) | 0.0% | (0/48) |
| | renal cell | | | | | | | | |
| | carcinoma | | | | | | | | |
| | Oncocytoma | 98.0% | (49/50) | 2.0% | (1/50) | 0.0% | (0/50) | 0.0% | (0/50) |
| Tumors of | Adenocarcinoma | 100.0% | (37/37) | 0.0% | (0/37) | 0.0% | (0/37) | 0.0% | (0/37) |
| the male | of the prostate | | | | | | | | |
| genital | Small cell | 93.8% | (15/16) | 0.0% | (0/16) | 6.3% | (1/16) | 6.3% | (1/16) |
| organs | neuroendocrine | | | | | | | | |
| | carcinoma of the | | | | | | | | |
| | prostate | | | | | | | | |
| | Seminoma | 80.0% | (40/50) | 18.0% | (9/50) | 2.0% | (1/50) | 0.0% | (0/50) |
| | Embryonal | 97.7% | (43/44) | 2.3% | (1/44) | 0.0% | (0/44) | 0.0% | (0/44) |
| | carcinoma of the | | | | | | | | |
| | testis | | | | | | | | |
| | Yolk sak tumor | 91.7% | (44/48) | 8.3% | (4/48) | 0.0% | (0/48) | 0.0% | (0/48) |
| | Teratoma | 96.3% | (26/27) | 3.7% | (1/27) | 0.0% | (0/27) | 0.0% | (0/27) |
| Tumors of | Adenoma of the | 100.0% | (50/50) | 0.0% | (0/50) | 0.0% | (0/50) | 0.0% | (0/50) |
| endocrine | thyroid gland | | | | | | | | |
| organs | Papillary thyroid | 97.8% | (45/46) | 2.2% | (1/46) | 0.0% | (0/46) | 0.0% | (0/46) |
| | carcinoma | | | | | | | | |
| | Follicular thyroid | 100.0% | (49/49) | 0.0% | (0/49) | 0.0% | (0/49) | 0.0% | (0/49) |
| | carcinoma | | | | | | | | |
| | Medullary thyroid | 97.9% | (47/48) | 2.1% | (1/48) | 0.0% | (0/48) | 0.0% | (0/48) |
| | carcinoma | | | | | | | | |
| | Anaplastic thyroid | 87.5% | (21/24) | 8.3% | (2/24) | 4.2% | (1/24) | 0.0% | (0/24) |
| | carcinoma | | | | | | | | |
| | Adrenal cortical | 100.0% | (48/48) | 0.0% | (0/48) | 0.0% | (0/48) | 0.0% | (0/48) |
| | adenoma | | | | | | | | |
| | Adrenal cortical | 88.0% | (22/25) | 8.0% | (2/25) | 4.0% | (1/25) | 4.0% | (1/25) |
| | carcinoma | | | | | | | | |
| | Phaeochromocytoma | 97.8% | (45/46) | 0.0% | (0/46) | 2.2% | (1/46) | 2.2% | (1/46) |
| | Neuroendocrine | 97.9% | (46/47) | 0.0% | (0/47) | 2.1% | (1/47) | 2.1% | (1/47) |
| | tumor (NET) | | | | | | | | |
| Tumors of | Hodgkin's | 97.7% | (42/43) | 2.3% | (1/43) | 0.0% | (0/43) | 0.0% | (0/43) |
| haematopoeitic | Lymphoma | | | | | | | | |
| and lymphoid | Non-Hodgkin's | 97.8% | (44/45) | 0.0% | (0/45) | 2.2% | (1/45) | 0.0% | (0/45) |
| tissues | Lymphoma | | | | | | | | |
| | Thymoma | 92.6% | (25/27) | 0.0% | (0/27) | 7.4% | (2/27) | 7.4% | (2/27) |
| Tumors of | Tenosynovial giant | 100.0% | (45/45) | 0.0% | (0/45) | 0.0% | (0/45) | 0.0% | (0/45) |
| soft tissue | cell tumor | | | | | | | | |
| and bone | Granular cell tumor | 100.0% | (28/28) | 0.0% | (0/28) | 0.0% | (0/28) | 0.0% | (0/28) |
| | Leiomyoma | 100.0% | (48/48) | 0.0% | (0/48) | 0.0% | (0/48) | 0.0% | (0/48) |
| | Leiomyosarcoma | 100.0% | (47/47) | 0.0% | (0/47) | 0.0% | (0/47) | 0.0% | (0/47) |
| | Liposarcoma | 97.9% | (46/47) | 2.1% | (1/47) | 0.0% | (0/47) | 0.0% | (0/47) |
| | Angiosarcoma | 90.0% | (27/30) | 6.7% | (2/30) | 3.3% | (1/30) | 3.3% | (1/30) |
| | Osteosarcoma | 90.0% | (18/20) | 0.0% | (0/20) | 10.0% | (2/20) | 5.0% | (1/20) |
| | Chondrosarcoma | 92.9% | (13/14) | 0.0% | (0/14) | 7.1% | (1/14) | 7.1% | (1/14) |

### 3.4 Extended MAGE-A1 Analysis in Breast and Lung Cancers

3.4.1 Breast Cancer Prognosis TMA (BRE 1.1)

MAGE-A1 staining was interpretable in 757 of the 849 (89%) arrayed breast cancers. Reasons for non-interpretable tumors included lack of tumors cells in the tissue spot or loss of tissues spots on the slide. A positive immunohistochemistry result was found in 11% of the cancers, including 7.5% cancers with ≥80%, positive tumor cells. No relevant associations were found between MAGE-A1 expression and histological subtype, tumor stage/grade, presence of lymph node/distant metastasis, molecular parameters (estrogen receptor, HER2 receptor) or patient survival (MAGE-A1 negative vs positive, p=0.0598). All data is summarized in Table 3.

TABLE 3
Relationship between MAGE-A1 expression and breast cancer phenotype.

| | | | | Samples | | Samples | | Samples | | |
|---|---|---|---|---|---|---|---|---|---|---|
| | | | | with less | | with 80- | | with | | |
| | | | | than 80% | | 100% | | 100% | | |
| | | Negative | | positive | | positive | | positive | | p- |
| | | samples | | tumor cells | | tumor cells | | tumor cells | | value |
| All tumors | | 89.3% | (676/757) | 3.2% | (24/757) | 7.5% | (57/757) | 7.1% | (54/757) | |
| Histology | NST | 87.7% | (407/464) | 4.3% | (20/464) | 8.0% | (37/464) | 7.5% | (35/464) | |
| | lobulary | 90.4% | (208/230) | 1.7% | (4/230) | 7.8% | (18/230) | 7.4% | (17/230) | 0.3280 * |
| | medullary | 92.3% | (12/13) | 0.0% | (0/13) | 7.7% | (1/13) | 7.7% | (1/13) | 0.7420 * |
| | mucinous | 100.0% | (8/8) | 0.0% | (0/8) | 0.0% | (0/8) | 0.0% | (0/8) | |
| | papillary | 100.0% | (2/2) | 0.0% | (0/2) | 0.0% | (0/2) | 0.0% | (0/2) | |
| | SQCC | 100.0% | (1/1) | 0.0% | (0/1) | 0.0% | (0/1) | 0.0% | (0/1) | |
| | tubulary | 96.2% | (25/26) | 0.0% | (0/26) | 3.8% | (1/26) | 3.8% | (1/26) | 0.3702 * |
| pT** | pT1 | 88.7% | (205/231) | 3.9% | (9/231) | 7.4% | (17/231) | 6.9% | (16/231) | 0.4766 |
| | pT2 | 89.2% | (174/195) | 3.1% | (6/195) | 7.7% | (15/195) | 7.2% | (14/195) | |
| | pT3 | 73.1% | (19/26) | 11.5% | (3/26) | 15.4% | (4/26) | 15.4% | (4/26) | |
| | pT4 | 75.0% | (9/12) | 16.7% | (2/12) | 8.3% | (1/12) | 8.3% | (1/12) | |
| pN** | pN0 | 88.7% | (250/282) | 3.9% | (11/282) | 7.4% | (21/282) | 7.4% | (21/282) | 0.1991 |
| | pN1 | 87.7% | (143/163) | 3.7% | (6/163) | 8.6% | (14/163) | 7.4% | (12/163) | |
| | pN2 | 73.7% | (14/19) | 15.8% | (3/19) | 10.5% | (2/19) | 10.5% | (2/19) | |
| pM** | pM0 | 88.2% | (402/456) | 4.4% | (20/456) | 7.5% | (34/456) | 7.2% | (33/456) | 0.0322 |
| | PM1 | 62.5% | (5/8) | 0.0% | (0/8) | 37.5% | (3/8) | 25.0% | (2/8) | |
| Grading** | Grade 1 | 89.1% | (57/64) | 3.1% | (2/64) | 7.8% | (5/64) | 7.8% | (5/64) | 0.6459 |
| | Grade 2 | 87.8% | (267/304) | 3.6% | (11/304) | 8.6% | (26/304) | 7.9% | (24/304) | |
| | Grade 3 | 86.5% | (83/96) | 7.3% | (7/96) | 6.3% | (6/96) | 6.3% | (6/96) | |
| ER score | negative | 92.7% | (101/109) | 1.8% | (2/109) | 5.5% | (6/109) | 4.6% | (5/109) | 0.1718 |
| (Allred)** | (0-2) | | | | | | | | | |
| | 3-4 | 100.0% | (9/9) | 0.0% | (0/9) | 0.0% | (0/9) | 0.0% | (0/9) | |
| | 5-6 | 96.6% | (28/29) | 0.0% | (0/29) | 3.4% | (1/29) | 3.4% | (1/29) | |
| | 7-8 | 85.0% | (261/307) | 5.9% | (18/307) | 9.1% | (28/307) | 8.8% | (27/307) | |
| HER2 | 0 | 86.7% | (189/218) | 3.7% | (8/218) | 9.6% | (21/218) | 9.2% | (20/218) | 0.3597 |
| (IHC)** | 1+ | 89.1% | (114/128) | 2.3% | (3/128) | 8.6% | (11/128) | 7.8% | (10/128) | |
| | 2+ | 83.8% | (31/37) | 13.5% | (5/37) | 2.7% | (1/37) | 2.7% | (1/37) | |
| | 3+ | 89.1% | (57/64) | 4.7% | (3/64) | 6.3% | (4/64) | 6.3% | (4/64) | |
| HER2 | amplification | 87.8% | (36/41) | 7.3% | (3/41) | 4.9% | (2/41) | 4.9% | (2/41) | 0.4505 |
| (FISH)** | Normal | 86.0% | (196/228) | 4.4% | (10/228) | 9.6% | (22/228) | 9.6% | (22/228) | |

* vs NST,
**NST only.

3.4.2 Lung Cancer Prognosis TMA (LUN 1.1)

MAGE-A1 staining was interpretable in 567 of the 616 (92%) arrayed lung cancers. Reasons for non-interpretable tumors included lack of tumors cells in the tissue spot or loss of tissue spots on the slide. A positive immunohistochemistry result was found in 18% of the cancers, including 9% cancers with ≥80% positive tumor cells. MAGE-A1 expression was significantly more frequent in squamous cell cancers (30%, including 16% cancers with ≥80% positive tumor cells) as compared to adenocarcinomas (7%, including 3% with ≥80% pos. tumor cells, p<0.0001, Table 4). No relevant associations were found between MAGE-A1 expression and tumor stage/grade, presence of lymph node/distant metastasis, UICC stage or patient survival (MAGE-A1 negative vs positive, p=0.2532), neither if all cancers were jointly analyzed (Table 4) nor in subset analyses of adeno- (Table 5) and squamous cell carcinomas (Table 6).

TABLE 4
Relationship between MAGE-A1 expression and lung cancer phenotype (all cancers).
ADC = adenocarcinoma, BAC = bronchioalveolar carcinoma, LCLC =
large cell lung cancer, SCLC = small cell lung cancer, SQCC = squamous
cell lung cancer, UICC = International Union against Cancer.

| | | | Samples | | Samples | | Samples | | |
|---|---|---|---|---|---|---|---|---|---|
| | | | with less | | with 80- | | with | | |
| | | | than 80% | | 100% | | 100% | | |
| | Negative | | positive | | positive | | positive | | |
| | samples | | tumor cells | | tumor cells | | tumor cells | | p-value |
| All tumors | 81.7% | (463/567) | 9.5% | (54/567) | 8.8% | (50/567) | 5.8% | (33/567) | |
| ADC | 92.7% | (179/193) | 4.1% | (8/193) | 3.1% | (6/193) | 2.6% | (5/193) | |
| BAC | 81.8% | (9/11) | 9.1% | (1/11) | 9.1% | (1/11) | 0.0% | (0/11) | |
| LCLC | 85.1% | (97/114) | 9.6% | (11/114) | 5.3% | (6/114) | 4.4% | (5/114) | 0.1993 * |
| SCLC | 95.2% | (20/21) | 4.8% | (1/21) | 0.0% | (0/21) | 0.0% | (0/21) | 0.7366 * |
| SQCC | 69.3% | (158/228) | 14.5% | (33/228) | 16.2% | (37/228) | 10.1% | (23/228) | <0.0001 * |
| pT1 | 89.2% | (140/157) | 7.0% | (11/157) | 3.8% | (6/157) | 2.5% | (4/157) | 0.0723 |
| pT2 | 77.6% | (236/304) | 11.2% | (34/304) | 11.2% | (34/304) | 6.9% | (21/304) | |
| pT3 | 76.0% | (38/50) | 10.0% | (5/50) | 14.0% | (7/50) | 12.0% | (6/50) | |
| pT4 | 88.0% | (44/50) | 6.0% | (3/50) | 6.0% | (3/50) | 4.0% | (2/50) | |
| pN0 | 81.8% | (198/242) | 9.1% | (22/242) | 9.1% | (22/242) | 6.2% | (15/242) | 0.8920 |
| pN1 | 80.5% | (223/277) | 10.5% | (29/277) | 9.0% | (25/277) | 5.4% | (15/277) | |
| pM0 | 81.6% | (438/537) | 9.5% | (51/537) | 8.9% | (48/537) | 6.1% | (33/537) | 0.1969 |
| pM1 | 83.3% | (25/30) | 10.0% | (3/30) | 6.7% | (2/30) | 0.0% | (0/30) | |
| Grade 1 | 91.7% | (11/12) | 8.3% | (1/12) | 0.0% | (0/12) | 0.0% | (0/12) | 0.2372 |
| Grade 2 | 78.3% | (253/323) | 10.2% | (33/323) | 11.5% | (37/323) | 6.8% | (22/323) | |
| Grade 3 | 84.0% | (142/169) | 9.5% | (16/169) | 6.5% | (11/169) | 5.3% | (9/169) | |
| Grade 4 | 86.8% | (33/38) | 10.5% | (4/38) | 2.6% | (1/38) | 2.6% | (1/38) | |
| UICC Ia | 87.0% | (80/92) | 8.7% | (8/92) | 4.3% | (4/92) | 3.3% | (3/92) | 0.4988 |
| UICC Ib | 81.6% | (102/125) | 8.0% | (10/125) | 10.4% | (13/125) | 7.2% | (9/125) | |
| UICC Iia | 91.7% | (22/24) | 4.2% | (1/24) | 4.2% | (1/24) | 0.0% | (0/24) | |
| UICC IIb | 73.6% | (81/110) | 14.5% | (16/110) | 11.8% | (13/110) | 7.3% | (8/110) | |
| UICC IIIa | 83.0% | (78/94) | 9.6% | (9/94) | 7.4% | (7/94) | 5.3% | (5/94) | |
| UICC IIIb | 86.0% | (49/57) | 5.3% | (3/57) | 8.8% | (5/57) | 7.0% | (4/57) | |
| UICC IV | 85.2% | (23/27) | 7.4% | (2/27) | 7.4% | (2/27) | 0.0% | (0/27) | |
| UICC X | 83.3% | (5/6) | 16.7% | (1/6) | 0.0% | (0/6) | 0.0% | (0/6) | |

* vs ADC

TABLE 5
Relationship between MAGE-A1 expression and lung
cancer phenotype (subset of adenocarcinomas).

| | | | Samples | | Samples | | Samples | | |
|---|---|---|---|---|---|---|---|---|---|
| | | | with less | | with 80- | | with | | |
| | | | than 80% | | 100% | | 100% | | |
| | Negative | | positive | | positive | | positive | | |
| | samples | | tumor cells | | tumor cells | | tumor cells | | p-value |
| All tumors | 92.7% | (179/193) | 4.1% | (8/193) | 3.1% | (6/193) | 2.6% | (5/193) | |
| pT1 | 97.1% | (66/68) | 2.9% | (2/68) | 0.0% | (0/68) | 0.0% | (0/68) | 0.3841 |
| pT2 | 92.5% | (86/93) | 4.3% | (4/93) | 3.2% | (3/93) | 2.2% | (2/93) | |
| pT3 | 81.3% | (13/16) | 6.3% | (1/16) | 12.5% | (2/16) | 12.5% | (2/16) | |
| pT4 | 85.7% | (12/14) | 7.1% | (1/14) | 7.1% | (1/14) | 7.1% | (1/14) | |
| pN0 | 91.1% | (82/90) | 4.4% | (4/90) | 4.4% | (4/90) | 3.3% | (3/90) | 0.6106 |
| PN1 | 94.9% | (74/78) | 2.6% | (2/78) | 2.6% | (2/78) | 2.6% | (2/78) | |
| pM0 | 92.6% | (163/176) | 4.0% | (7/176) | 3.4% | (6/176) | 2.8% | (5/176) | 0.6454 |
| pM1 | 94.1% | (16/17) | 5.9% | (1/17) | 0.0% | (0/17) | 0.0% | (0/17) | |
| Grade 1 | 100.0% | (4/4) | 0.0% | (0/4) | 0.0% | (0/4) | 0.0% | (0/4) | 0.5853 |
| Grade 2 | 93.3% | (112/120) | 3.3% | (4/120) | 3.3% | (4/120) | 2.5% | (3/120) | |
| Grade 3 | 92.9% | (52/56) | 3.6% | (2/56) | 3.6% | (2/56) | 3.6% | (2/56) | |
| Grade 4 | 66.7% | (4/6) | 33.3% | (2/6) | 0.0% | (0/6) | 0.0% | (0/6) | |
| UICC Ia | 97.5% | (39/40) | 2.5% | (1/40) | 0.0% | (0/40) | 0.0% | (0/40) | 0.9027 |
| UICC Ib | 88.9% | (40/45) | 6.7% | (3/45) | 4.4% | (2/45) | 2.2% | (1/45) | |
| UICC Iia | 90.9% | (10/11) | 9.1% | (1/11) | 0.0% | (0/11) | 0.0% | (0/11) | |
| UICC IIb | 91.7% | (22/24) | 0.0% | (0/24) | 8.3% | (2/24) | 8.3% | (2/24) | |
| UICC IIIa | 93.9% | (31/33) | 3.0% | (1/33) | 3.0% | (1/33) | 3.0% | (1/33) | |
| UICC IIIb | 88.2% | (15/17) | 5.9% | (1/17) | 5.9% | (1/17) | 5.9% | (1/17) | |
| UICC IV | 93.3% | (14/15) | 6.7% | (1/15) | 0.0% | (0/15) | 0.0% | (0/15) | |
| UICC X | 100.0% | (3/3) | 0.0% | (0/3) | 0.0% | (0/3) | 0.0% | (0/3) | |

TABLE 6
Relationship between MAGE-A1 expression and lung cancer
phenotype (subset of squamous cell carcinomas).

| | | | Samples | | Samples | | Samples | | |
|---|---|---|---|---|---|---|---|---|---|
| | | | with less | | with 80- | | with | | |
| | | | than 80% | | 100% | | 100% | | |
| | Negative | | positive | | positive | | positive | | |
| | samples | | tumor cells | | tumor cells | | tumor cells | | p-value |
| All tumors | 69.3% | (158/228) | 14.5% | (33/228) | 16.2% | (37/228) | 10.1% | (23/228) | |
| pT1 | 77.8% | (35/45) | 13.3% | (6/45) | 8.9% | (4/45) | 4.4% | (2/45) | 0.8199 |
| pT2 | 65.7% | (90/137) | 14.6% | (20/137) | 19.7% | (27/137) | 12.4% | (17/137) | |
| pT3 | 66.7% | (16/24) | 16.7% | (4/24) | 16.7% | (4/24) | 12.5% | (3/24) | |
| pT4 | 78.9% | (15/19) | 10.5% | (2/19) | 10.5% | (2/19) | 5.3% | (1/19) | |
| pN0 | 65.5% | (57/87) | 16.1% | (14/87) | 18.4% | (16/87) | 12.6% | (11/87) | 0.4717 |
| PN1 | 72.5% | (95/131) | 13.7% | (18/131) | 13.7% | (18/131) | 6.9% | (9/131) | |
| pM0 | 69.9% | (153/219) | 14.2% | (31/219) | 16.0% | (35/219) | 10.5% | (23/219) | 0.1813 |
| PM1 | 55.6% | (5/9) | 22.2% | (2/9) | 22.2% | (2/9) | 0.0% | (0/9) | |
| Grade 1 | 100.0% | (3/3) | 0.0% | (0/3) | 0.0% | (0/3) | 0.0% | (0/3) | 0.7600 |
| Grade 2 | 68.1% | (126/185) | 15.7% | (29/185) | 16.2% | (30/185) | 9.7% | (18/185) | |
| Grade 3 | 71.8% | (28/39) | 10.3% | (4/39) | 17.9% | (7/39) | 12.8% | (5/39) | |
| Grade 4 | — | (0/0) | — | (0/0) | — | (0/0) | — | (0/0) | |
| UICC Ia | 69.2% | (18/26) | 19.2% | (5/26) | 11.5% | (3/26) | 7.7% | (2/26) | 0.6184 |
| UICC Ib | 63.6% | (28/44) | 11.4% | (5/44) | 25.0% | (11/44) | 18.2% | (8/44) | |
| UICC Iia | 88.9% | (8/9) | 0.0% | (0/9) | 11.1% | (1/9) | 0.0% | (0/9) | |
| UICC IIb | 66.2% | (43/65) | 18.5% | (12/65) | 15.4% | (10/65) | 7.7% | (5/65) | |
| UICC IIIa | 78.0% | (32/41) | 12.2% | (5/41) | 9.8% | (4/41) | 7.3% | (3/41) | |
| UICC IIIb | 72.2% | (13/18) | 11.1% | (2/18) | 16.7% | (3/18) | 11.1% | (2/18) | |
| UICC IV | 62.5% | (5/8) | 12.5% | (1/8) | 25.0% | (2/8) | 0.0% | (0/8) | |
| UICC X | 50.0% | (1/2) | 50.0% | (1/2) | 0.0% | (0/2) | 0.0% | (0/2) | |

### 3.5. MAGE-A1 Heterogeneity Analysis

In order to better understand intra-tumoral heterogeneity of MAGE-A1 expression and MAGE-A1 expression in lymph node and distant metastases, additional TMAs were analyzed that consisted of multiple spots taken from the same tumor and tissue from lymph node and distant metastasis. For this analysis, a tumor was considered i) homogeneously positive when all interpretable tissue spots had at least 5% positive tumor cells, ii) negative when all tissue spots had a negative IHC result iii) and heterogeneously positive when positive and negative tissue spots were present in the same tumor.

3.5.1 Bladder Cancer Heterogeneity TMA (BLA 1.2)

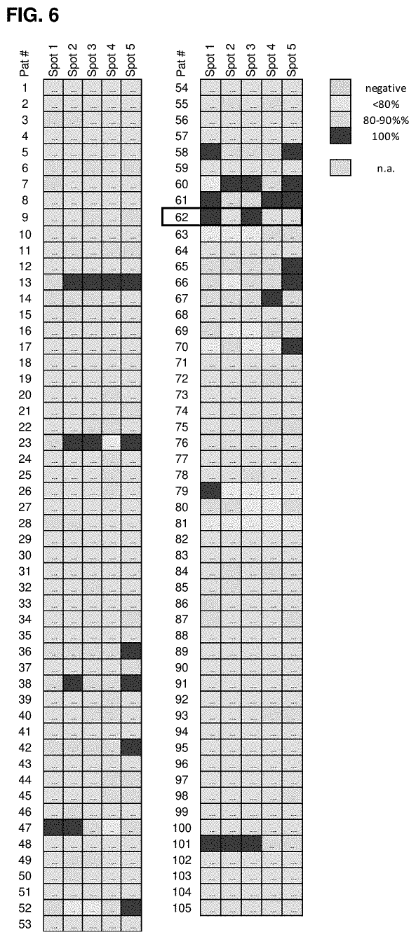

The TMA was made from each 5 samples of 105 muscle invasive (pT2-4) bladder cancers. All 5 spots were interpretable in n=62 cancers, 4 spots in n=25 cancers, 3 spots in n=14 cancers and 2 spots in n=4 cancers. The detailed staining results are given in FIG. 6.
20 (19%) of the 105 cancers had a positive result in all interpretable tissue spots and where considered “homogeneously positive”. These included 15 cancers with (typically) ≥80% positive tumor cells and 5 with (typically)<80%.

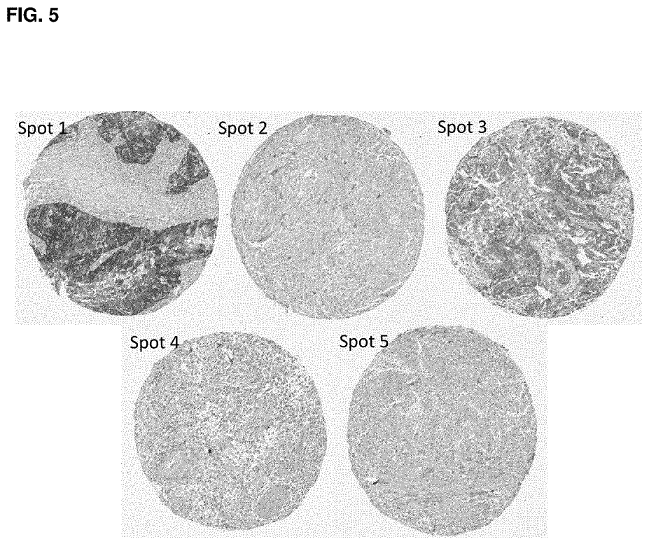

1 cancer (1%) had massive staining differences across the spots (negative and 100% positive spots, see FIG. 5) and was considered heterogeneously positive.

3.5.2 Esophageal Cancer Prognosis TMA (ESO 1.1)

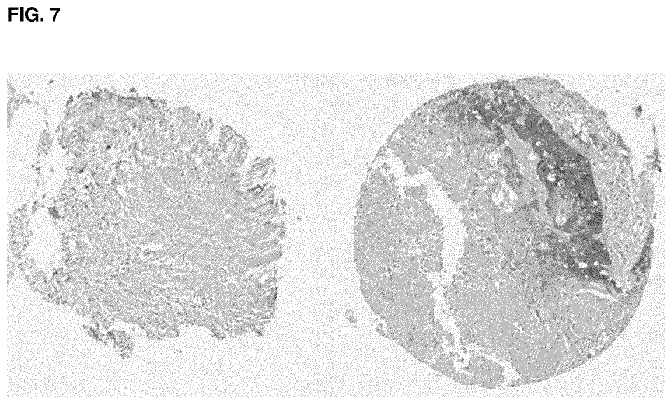

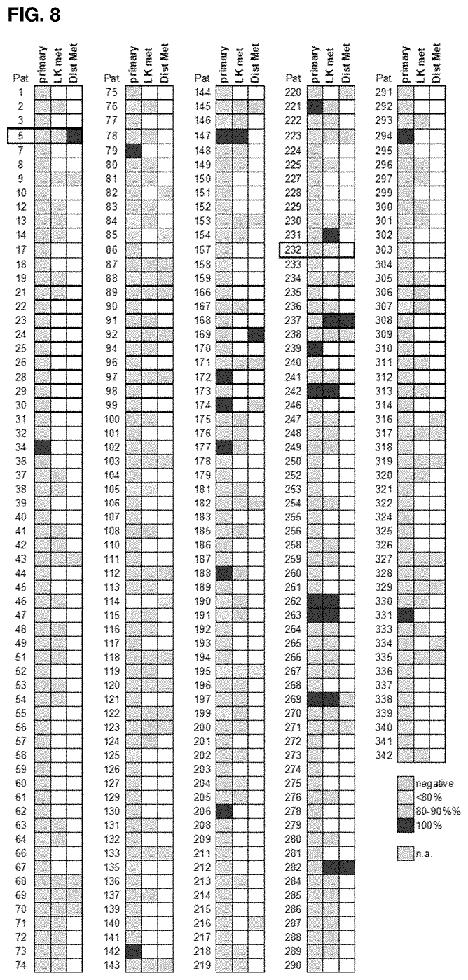

The TMA was made from up to each 3 samples (including primary tumor, lymph node metastasis and distant metastasis) of 342 esophageal cancers. 263 (76.9%) cancers were interpretable for MAGE-A1 staining. The MAGE-A1 staining results are summarized in FIG. 8.
Positive MAGE-A1 staining was found in 50 (19%) of these 263 tumors. These included 22 cancers with 100% positive tumor cells, 7 with 80-90%, and 21 with <80%.
At least 2 interpretable tissue spots per cancer (i.e., primary tumor and lymph node metastasis or primary tumor and distant metastasis) were available from 78 cancers. 16/78 (21%) cancers were MAGE-A1 positive. Of these, 14 cancers had comparable percentages of MAGE-A1 positive tumor cells in the primary cancer and metastatic tissue spots. Only 2 cancers showed evidence for heterogeneity between the primary tumor and the metastasis: Pat 5 (FIG. 7) had a negative primary tumor spot but a 100% MAGE-A1 positive distant metastasis, and Pat 232 had a negative primary tumor spot but a <80% MAGE-A1 positive lymph node metastasis.

3.5.3 Lung Cancer Heterogeneity TMA (LUN 4.2)

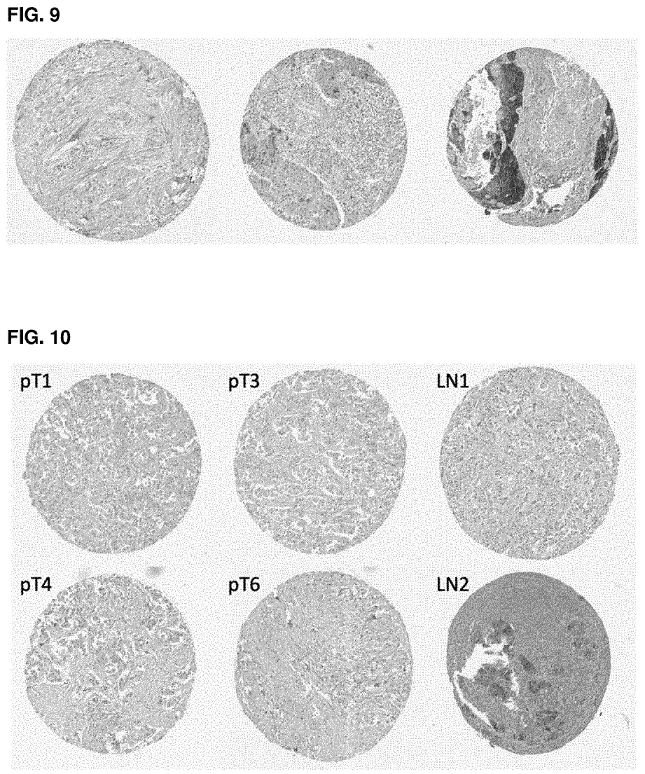

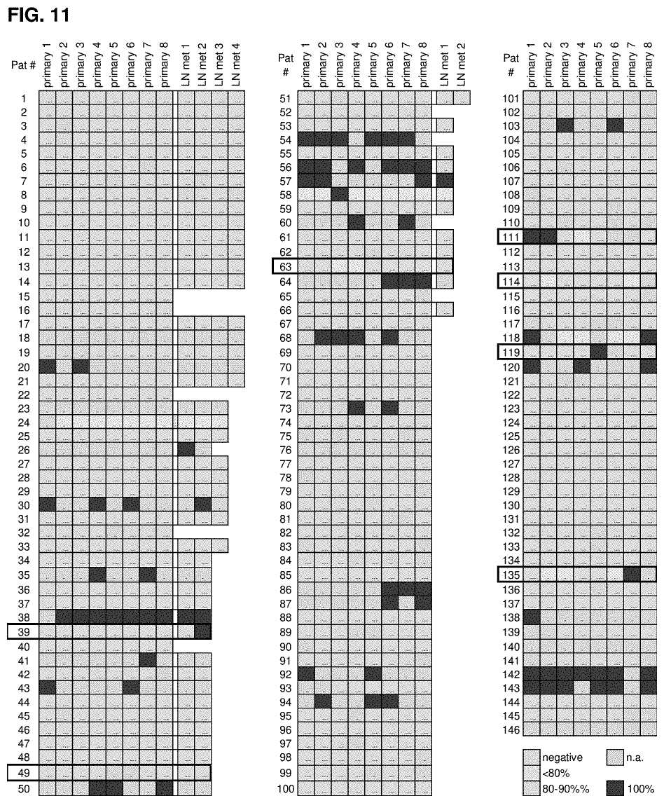

The TMA was made from 8 samples of the primary tumor and up to 4 samples from the lymph node metastases of 146 lung cancers. All 146 cancers were interpretable by IHC. The MAGE-A1 staining results are summarized in FIG. 11.
In the primary cancers, positive MAGE-A1 staining was found in 38 tumors (26%), including 32 (22%) cancers with homogenous staining of ≥80% of tumor cells in all tissue spots and 6 cancers (4%) with heterogenous MAGE-A1 staining ranging from negative to 100% positive tumor cells (see Pat. 49, 63, 111, 114, 119 and 135 in FIG. 9 and FIG. 11).
Lymph node metastases usually showed comparable levels of MAGE-A1 expression as the corresponding primary cancers: All 14 MAGE-A1 primary cancers with homogeneous MAGE-A1 positivity (≥80% of tumor cells positive) had also homogeneously positive lymph node metastasis (≥80% of tumor cells positive). However, 2 heterogeneously positive primary cancers had MAGE-A1 negative lymph node metastases (Pat. 49, 63). Of 108 MAGE-A1 negative primary cancers, only one cancer had a MAGE-A1 positive lymph node metastasis (Pat. 39, FIG. 10).

3.5.4. Stomach Cancer Heterogeneity TMA (STO 2.2)

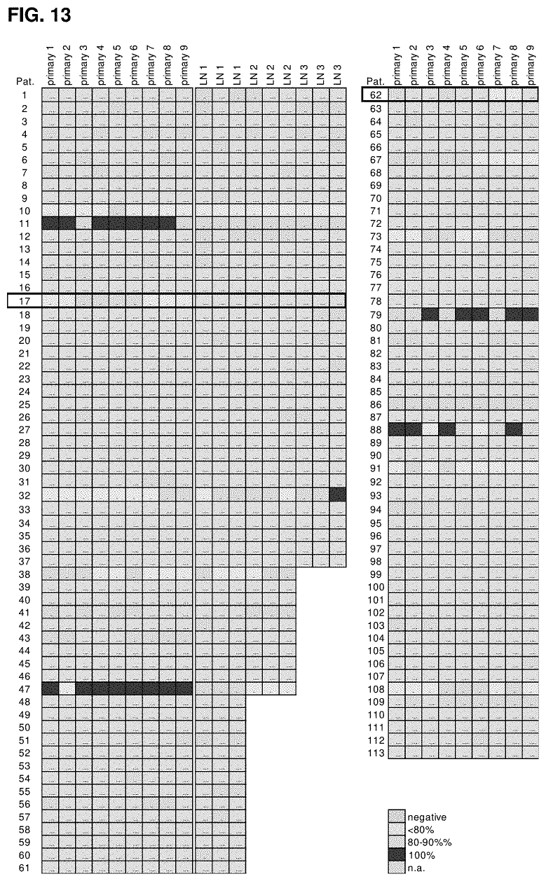

The TMA was made from each 9 samples of the primary tumor and each 3 samples of up to 3 lymph node metastases of 113 stomach cancers. 37 cancers had 3 lymph node metastases, 10 cancers had 2 lymph node metastases and 14 cancers had 1 lymph node metastasis. In case of the remaining 52 cancers only the primary tumor was available. The MAGE-A1 staining results are summarized in FIG. 13.

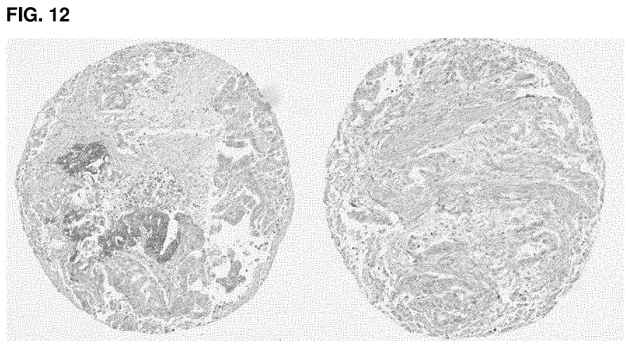

In the primary cancers, positive MAGE-A1 staining was found in 14 tumors (12.4%), including 13 (11.5%) cancers with homogenous staining. Eight (7%, Pat. 10, 17, 32, 38, 67, 73, 91, 108) cancers had <80% positive tumor cells, 5 (4%, Pat. 11, 47, 79, 86, 88) had ≥80% positive tumor cells. Only one cancer (Pat. 62) showed signs of heterogeneity with both negative and positive tumor spots (FIG. 12).

Lymph node metastases usually showed comparable levels of MAGE-A1 expression as the corresponding primary cancers: For six of the MAGE-A1 positive primary tumors lymph nodes metastases samples existed. In five of these cases also the lymph node metastases were homogeneously positive with a comparable fraction of MAGE-A1 positive tumor cells. In the remaining patient lymph node metastases were all MAGE-A1 negative, even though the primary tumor was homogenously MAGE-A1 positive. For MAGE-A1 negative primary cancers all available lymph node metastases were also MAGE-A1 negative.

Summary of the Heterogeneity Analyses

An overview on the cancers with heterogenous MAGE-A1 immunostaining that have been identified in this study is given in Table 6. Positive cancers typically had a positive result in all different analyzed areas of the primary tumor bulk, often maintaining a high fraction of positive tumor cells. For example, 15 of 21 MAGE-A1 positive urinary bladder cancers had ≥80% positive tumor cells in the vast majority of analyzed samples, and only 1 cancer had both negative and positive tumor areas. In esophageal squamous cell cancers, 29 of 50 MAGE-A1 positive cancers had ≥80% positive tumor cells in the vast majority of analyzed samples, and only 2 cancer had both negative and positive tumor areas. The highest heterogeneity was found in lung squamous cell carcinomas, where 6/38 MAGE-A1 positive cancers had tumor areas completely lacking detectable MAGE-A1 expression.

TABLE 6
Number of MAGE-A1 positive cancers and fraction of cancers with
heterogenous MAGE-A1 staining. a) Percentage of MAGE-A1 positive cancers.

| | | | | MAGE-A1 | |
|---|---|---|---|---|---|
| | | | | heterogenous n (%)^{a} | |
| | | | | | between |
| | | | | within the | primary |
| | | # of | MAGE- | primary | cancer and |
| TMA | Format | cancers | A1 pos | cancer | metastases |
| BLA | 5 spots per pri. | 105 | 20 | 1 (5%) | — |
| 1.2 | | | (19%) | | |
| ESO | ≤3 spots from matched pri., LN met., | 78 | 16 | — | 2 (13%) |
| 1.1 | dist met. | | (21%) | | |
| LUN | 8 spots per pri. | 146 | 38 | 6 (16%) | — |
| 4.2 | | | (26%) | | |
| LUN | 8 spots per pri. + ≤4 matched LN met. | 57 | 17 | — | 3 (18%) |
| 4.2 | | | (30%) | | |
| STO | 9 spots per pri. | 113 | 14 | 1 (7%) | — |
| 2.2 | | | (12%) | | |
| STO | 9 spots per pri. + 3 matched LN met. | 61 | 6 | — | 1 (17%) |
| 2.2 | | | (10%) | | |

### 3.5 Summary and Conclusions

More than 8,300 tissue samples from >5,400 individual cancers representing more than 80 tumor types and virtually all human normal tissues were analyzed for MAGE-A1 immunostaining using the antibody MA454. A relatively high antibody concentration (8 μg/ml) was chosen for this study in order to detect MAGE-A1 with the highest possible sensitivity. This was possible because the antibody did not show relevant non-specific background staining under this condition.

Based on the analysis of other proteins of the MAGE-A family, the antibody 4545 used in this study was highly specific for MAGE-A1. This is also supported by the normal tissue analysis, which revealed positive staining only in the expected tissue type (testis). The fact that no other normal tissues showed MAGE-A1 expression is a relevant finding with respect to anti-cancer therapies targeting MAGE-A1 positive cancer.

Successful analysis of more than 2,900 cancer biopsies of the multitumor-array identified tumor types with frequent MAGE-A1 expression such as malignant melanoma (36% positive), squamous cell carcinomas of the lung (27%), esophagus (26%) and vagina (24%), muscle invasive bladder cancer (26%), adenocarcinoma of the esophagus (24%), tubular breast cancer (23%) and seminoma (20%). However, in some of these tumor types, MAGE-A1 expression was often seen only in less than 80% of the tumor cells. Most homogeneous staining (≥80% of tumor cells expressing MAGE-A1) was found (for example) in breast cancers, squamous cell carcinomas of the lung, anal canal, and esophagus, muscle invasive bladder cancer as well as in gastric adenocarcinomas, chondro- and osteosarcomas, malignant mesotheliomas and pancreatic adenocarcinomas.

To increase the cohort size in breast and lung cancers, additional TMAs were analyzed. It showed that the fraction of cancers with detectable MAGE-A1 expression was about 10% in breast cancers and lung adenocarcinomas, but 30% in lung squamous cell cancers. Homogeneous expression (≥80% of tumor cells positive) was seen in 8% of breast cancers and 3% of lung adenocarcinomas, but in 16% of lung squamous cell carcinomas.

Heterogeneity analysis in cancers of the urinary bladder, esophagus, lung and stomach did reveal only minor heterogeneity within the primary cancers or between primary cancers and their metastases.

In summary, the results of this study confirm that MAGE-A1 expression occurs in a wide range of human cancers. The study identified several major tumor types with significant MAGE-A1 expression levels, such as melanoma, squamous non-small cell lung cancer, esophageal, gastric, breast [ductal, tubular, medullary], ovarian, mesothelioma, bladder, anal, sarcomas, primitive neuroectodermal, and various other solid tumors. MAGE-A1 Expression was detected to be present in distinct subpopulations in the 80-100% range of all tumor cells in above tumor types. Tumor heterogeneity analyses and comparison of primary vs. metastatic/lymph node lesions demonstrated consistency of MAGE-A1 expression across the vast majority of primary tumors and metastases.

Thus, from these results it is evident that determining by means of the antibody MA454 in a tumor cell sample obtained from a patient the fraction of cells that express MAGE-A1 and applying a threshold as described herein (for example, if a fraction of at least about 30%, about 40%, about 50%, about 60%, about 70% or about at least 80% of the cells of the tumor sample are found to express MAGE-A1) is highly suitable for selecting patients being diagnosed with a solid tumor for subsequent treatment of the solid tumor by an adoptive cell therapy agent as described here. Likewise, it is also evident that the antibody M454 is equally suitable for being used in a corresponding method of treatment of MAGE-A1 positive solid tumors and a method of predicting whether a patient being diagnosed with a MAGE-A1 solid tumor will be responsive to treatment of this tumor.

### Example 2: Clinical Trial Protocol for Treatment of Patients with the Adoptive Cell Therapy Product

The target population for the treatment with are patients with HLA-A*02:01 genotype and advanced-stage/metastatic, MAGE-A1+ solid tumors that either have no further approved therapeutic alternative(s) or are in a non-curable state and have received a minimum of two lines of systemic therapy.

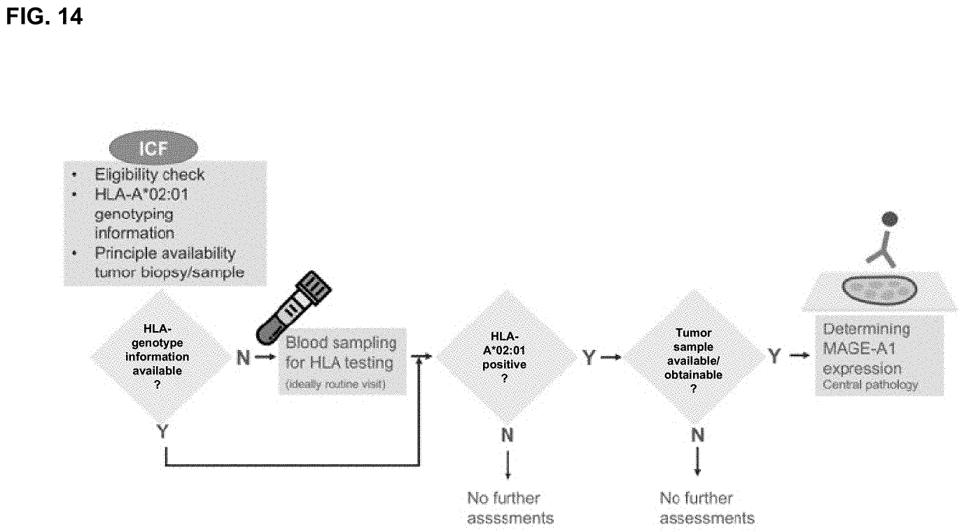

In accordance with the flowchart of FIG. 14, patients diagnosed with a solid tumor who are considered to receive treatment, for example, with an adoptive cell therapy agent such as chimeric antigen receptor T-cell (CAR T-cell) or a genetically modified T-cell that expresses a T cell receptor that specifically binds MAGE 1A are subjected to an eligibility test by determining their HLA genotype (HLA-A*02:01 in this illustrative example) and determining the availability of a tumor cell sample of the solid tumor. If the patient is HLA-A*02:01 positive and a tumor cell sample can be provided, the fraction of the cells of the tumor cell sample that is found to express MAGE-A1 is determined as described herein. For each biopsy, a paraffine-embedded tumor sample will be processed and 4 microtome sections fixated on slides will be used for analysis, i.e determination of the fraction of MAGE A1 positive cells in the tumor cell sample. In case the fraction of MAGE-A1 positive cells meets or exceeds a predetermined threshold, the patient will be selected for treatment of the solid tumor.

Inclusion Criteria:

The following inclusion criteria are used to for a patient to be able to enroll in this trial/treatment:
1. Age≥18 years
2. Presence of advanced-stage/metastatic, solid tumor malignancy for which there is no further available approved therapeutic alternative(s) or which is in a non-curable state as per treating physician's assessment with the patient having received a minimum of two lines of systemic therapy.
3. HLA-A*02:01 genotype.
4. MAGE-A1+ tumor with ≥80% of tumor cells positive for MAGE-A1 as per immunohistochemistry.
5. As per most recent tumor assessment, presence of radiologically measurable disease—with at least 1 lesion, not previously irradiated, that can be accurately measured as per RECIST Version 1.1 with computed tomography (CT) or magnetic resonance imaging (MRI).
6. Eastern Cooperative Oncology Group (ECOG) performance status 1.
7. Life expectancy>3 months as assessed by the Investigator.
8. Adequate organ function, defined as:
a. Bone marrow function: hemoglobin≥10 g/dL (equal to 6.2 mmol/L); platelet count≥100×10⁹/L; leukocyte count≥3.0×10⁹/L.
b. Hepatic function: aspartate transaminase (AST) and alanine transaminase (ALT)≤3.0× upper limit of normal (ULN); bilirubin≥2.0×ULN.
c. Renal function: serum creatinine<1.5×ULN and/or creatinine clearance≥50 mL/min (Cockcroft-Gault equation).
d. International normalized ratio (INR)<1.5 and partial thromboplastin time (PTT) within 1.25× of upper and lower limit of normal.

The active ingredient in this trial are autologous CD8+ T-cells transduced with a MAGE-A1-directed TCR. The TCR may be a TCR having an alpha chain comprising an alpha chain comprising the CDR sequences shown in SEQ ID NO: 2 (Ile Phe Ser Asn Met Asp Met), SEQ ID NO: 3 (Ile Phe Ser Asn Met Asp Met) and SEQ ID NO: 4 (Cys Ala Glu Ser Ile Gly Ser Asn Ser Gly Tyr Ala Leu Asn Phe); and a beta chain comprising the CDR sequences shown in SEQ ID NO: 5 (Met Asp His Glu Asn), SEQ ID NO: 6 (Ser Tyr Asp Val Lys Met), and SEQ ID NO: 7 (Cys Ala Ser Arg Gly Leu Ala Gly Tyr Glu Gln Tyr Phe).

The adoptive cell therapy agent (product) is: Melanoma-associated antigen 1 (MAGE-A1) T-cell receptor (TCR)-transduced autologous cluster of differentiation (CD)8+ T-cells that are capable of eliminating MAGE-A1 epitope presenting tumor cells in patients with the human leukocyte antigen (HLA)-A*02:01 genotype. The product is provided as a cryopreserved, sterile, aqueous suspension for intravenous infusion without predilution or wash composed of the drug substance (viable transduced CD8+ T-cells) and the pharmaceutically acceptable cryopreservation excipients. Each dose (approximately 50 mL) is cryopreserved with a dose of 1×10⁸ or 1×10⁹ or 3×10⁹ viable transduced CD8+ T-cells and upon thawing should contain an acceptable range of viable transduced CD8+ T-cells expressing the MAGE-A1 TCR (counted by flow cytometry) in 2% HSA and 9% DMSO, in a 250 mL Cryobag.

The doses of the TCR that will be administered are as follows:
DL1: Fixed dose of 1×10⁸ cells transduced with the selected TCR
DL2: Fixed dose of 1×10⁹ cells transduced with the selected TCR
DL3: 3 to 9×10⁹ cells transduced with the selected TCR per patient

The dose administered on dose level 3 (DL 3) may depend on the total cell number that will be produced for the respective patient (between 3 to 9×10⁹ cells). If deemed necessary and approved by respective authorities, the maximum dose of the cells transduced with the selected TCR can exceed 9×10⁹ transduced.

The infusion of the transduced cells will take place over 5 to 15 minutes (maximum). Use of a central venous line for infusion is preferable, but not mandatory. Use of a parallel saline infusion is permitted.

The Treatment Period will start on Day 0 (day of administration of the adoptive cell therapy agent (product). Tumor status assessments will be performed at Day 42, Day 84, Day 126, Day 168, Day 224, Day 280, Day 364 and annually thereafter until Year 5 or until disease progression. In case of a partial response (PR) or a complete response (CR), a confirmative MRI-CT will be done within 4 weeks, with the same method used for the initial assessment as required by RECIST criteria, as an unscheduled visit. Tumor response will be assessed using RECIST Version 1.1 and iRECIST criteria. To explore the anti-tumor activity of the adoptive cell therapy agent, tumor lesions will be evaluated by tumor assessments/imaging. Cutaneous metastases are permissible provided they are documented with dated photographs and skin rulers. Disease-specific tumor status (e.g., for melanoma cutaneous lesions evaluated by photography) will be assessed according to RECIST 1.1 and iRECIST.

The invention is also characterized by the following items:

1. A method of selecting a patient for treatment of a solid tumor, wherein cells of the tumor express human melanoma associated antigen 1 (MAGE-A1) (e.g., UniProtKB accession number P43355 (MAGA1_HUMAN), the method comprising determining in a tumor cell sample obtained from the patient the fraction of cells that express MAGE-A1, wherein a patient is selected for treatment if a fraction of at least 30% of the cells of the tumor sample are found to express MAGE-A1.
2. The method of item 1, wherein the patient has been diagnosed with a corresponding HLA genotype and/or advanced-Stage metastatic solid tumor that express MAGE-A1.
3. The method of items 1 or 2, wherein the tumor cell sample from is obtained from a tumor selected from the group consisting of melanoma, lung cancer, esophageal cancer, gastric cancer, breast cancer, ovarian cancer, mesothelioma cancer, bladder cancer, anal cancer, chondrosarcoma cancer, osteosarcoma cancer, sarcoma cancer, adenoma cancer, primitive neuroectodermal cancer (primitive neuroectodermal tumor (PNET), and combinations thereof.
4. The method of item 3, wherein the lung cancer is selected from the group consisting of non-small cell lung cancer (NSCLC), including squamous cell carcinoma of the lung, adenocarcinoma of the lung, large cell carcinoma of the lung and other histologic types of NSCLC) and small cell lung cancer.
5. The method of items 3 or 4, wherein the breast cancer is selected from the group consisting of ductal breast cancer, ductal-invasive breast cancer, invasive breast cancer, tubular breast cancer, medullary breast cancer and combinations thereof.
6. The method of any one of items 3 to 5, wherein the gastric cancer is gastric adenocarcinoma and squamous cell cancer.
7. The method of any one of items 3 to 6, wherein the sarcoma cancer is selected from the group consisting of chondrosarcoma cancer, osteosarcoma cancer and combinations thereof.
8. The method of any one of items 3 to 7, wherein the cancer is selected from the group consisting of gastric adenocarcinoma, pancreatic adenocarcinoma and combinations thereof.
9. The method of any of the foregoing items, wherein the expression of MAGE-A1 in the tumor cell sample is determined by an immunohistochemistry method.
10. The method of item 9, wherein the immunohistochemistry method is immunostaining.
11. The method of item 10, wherein the tumor cell sample is embedded in paraffine prior to immunostaining.
12. The method of items 10 or 11, wherein the immunostaining is carried out with an antibody molecule that specifically binds MAGE-A1 or with a proteinaceous binding molecule with antibody-like binding properties that specifically binds MAGE-A1.
13. The method of item 12, wherein the antibody molecule specifically that binds MAGE-A1 is selected from the group consisting of a polyclonal antibody, a monoclonal antibody, a divalent antibody fragment, and a monovalent antibody fragment.
14. The method of item 13, wherein the antibody molecule is the monoclonal IgG1 mouse antibody MA454 or a fragment of the antibody MA454.
15. The method of item 12, wherein the proteinaceous binding molecule with antibody-like binding 25 properties that specifically binds MAGE-A1 is selected from the group consisting of an aptamer, a mutein based on a polypeptide of the lipocalin family, a glubody, a protein based on the ankyrin scaffold, a protein based on the crystalline scaffold, an adnectin, and an avimer.
16. The method of any of the foregoing items, wherein a patient is selected for treatment if a fraction 30 of at least about 50% of the cells, of least about 60% of the cells, of least about 65%, least about 70% of the cells, of at least about 75% of the cells, of at least about 80% of the cells, of at least about 90% of the cells, of at least about 95% of the cells, of at least about 96% of the cells, of at least about 97% of the cells, of at least about 98% of the cells, of at least about 99% of the cells or 100% of the cells of the tumor sample are found to express MAGE-A1.
17. The method of item 16, wherein if at least tumor cell samples are obtained from different parts of the tumor and at least one these tumor cell samples is found to express MAGE-A1 in a fraction of at least 30% of the cells of the tumor sample, the patient is selected for treatment.
18. The method of item 16 or 17, wherein a patient is selected for treatment if a fraction of at least about 50% of the cells, of least about 60% of the cells, of least about 65%, least about 70% of the cells, of at least about 75% of the cells, of at least about 80% of the cells, of at least about 90% of the cells, of at least about 95% of the cells, of at least about 96% of the cells, of at least about 97% of the cells, of at least about 98% of the cells, of at least about 99% of the cells or 100% of the cells of the tumor sample are found to express MAGE-A1.
19. The method of any of the forgoing items, wherein the treatment is immunotherapy.
20. The method of item 19, wherein the immunotherapy is by administration cells selected from the group consisting of autologous patient-derived T cells, allogeneic T cells and NK cells.
21. The method of item 20, wherein the T cells are selected from the group consisting of CD8+ T cells, CD4+ T cells, a combination thereof and T cells of other phenotypes.
22. The method of item 20 or 21, wherein the autologous T cells express a (recombinant) T cell receptor that specifically binds MAGE A1.
23. The method of item 22, wherein the T-cell receptor comprises an alpha chain comprising the CDR sequences shown in SEQ ID NO: 2 (Ile Phe Ser Asn Met Asp Met), SEQ ID NO: 3 (Ile Phe Ser Asn Met Asp Met) and SEQ ID NO: 4 (Cys Ala Glu Ser Ile Gly Ser Asn Ser Gly Tyr Ala Leu Asn Phe); and a beta chain comprising the CDR sequences shown in SEQ ID NO: 5 (Met Asp His Glu Asn), SEQ ID NO: 6 (Ser Tyr Asp Val Lys Met), and SEQ ID NO: 7 (Cys Ala Ser Arg Gly Leu Ala Gly Tyr Glu Gln Tyr Phe);
an alpha chain comprising the CDR sequences shown in SEQ ID NO: 8 (Asp Ser Ala Ser Asn Tyr), SEQ ID NO: 9 (Ile Arg Ser Asn Val Gly Glu), and SEQ ID NO: 10 (Cys Ala Ala Arg Pro Asn Ser Gly Asn Thr Pro Leu Val Phe); and a beta chain comprising the CDR sequences shown in SEQ ID NO: 11 (Ser Gln Val Thr Met), SEQ ID NO: 12 (Ala Asn Gln Gly Ser Glu Ala), and 13 (Cys Ser Val Glu Gln Asp Thr Asn Thr Gly Glu Leu Phe Phe);
an alpha chain comprising the CDR sequences shown in SEQ ID NO: 14 (Asn Ser Ala Phe Gln Tyr), SEQ ID NO: 15 (Thr Tyr Ser Ser Gly Asn), and SEQ ID NO: 16 (Cys Ala Met Ser Asp Thr Gly Asn Gln Phe Tyr Phe); and a beta chain comprising the CDR sequences shown in SEQ ID NO: 17 (Pro Arg His Asp Thr), SEQ ID NO: 18 (Phe Tyr Glu Lys Met Gln), and SEQ ID NO: 19 (Cys Ala Ser Ser Phe Arg Gly Gly Gly Ala Asn Val Leu Thr Phe);
an alpha chain comprising the CDR sequences shown in SEQ ID NO: 20 (Thr Ile Ser Gly Thr Asp Tyr), SEQ ID NO: 21 (Gly) and SEQ ID NO: 22 (Cys Ile Leu Phe Asn Phe Asn Lys Phe Tyr Phe); and a beta chain comprising the CDR sequences shown in SEQ ID NO: 23 (Leu Asn His Asn Val), SEQ ID NO: 24 (Tyr Tyr Asp Lys Asp Phe), and SEQ ID NO: 25 (Cys Ala Thr Ser Ser Gly Glu Thr Asn Glu Lys Leu Phe Phe);
an alpha chain comprising the CDR sequences shown in SEQ ID NO: 26 (Asp Ser Ala Ser Asn Tyr), SEQ ID NO: 27 (Ile Arg Ser) and SEQ ID NO: 28 (Cys Ala Ala Ser Pro Thr Gly Gly Tyr Asn Lys Leu Ile Phe); and a beta chain comprising the CDR sequences shown in SEQ ID NO: 29 (Met Asn His Glu Tyr), SEQ ID NO: 30 (Ser Val Gly Ala Gly Ile), and SEQ ID NO: 31 (Cys Ala Ser Ser Leu Gly Gly Ala Ser Gln Glu Gln Tyr Phe); or
an alpha chain comprising the CDR sequences shown in SEQ ID NO: 32 (Thr Ser Glu Ser Asn Tyr Tyr), SEQ ID NO: 33 (Gln Glu Ala Tyr) and SEQ ID NO: 34 (Cys Ala Phe Gly Tyr Ser Gly Gly Gly Ala Asp Gly Leu Thr Phe); and a beta chain comprising the CDR sequences shown in SEQ ID NO: 35 (Ser Gly His Asp Thr), SEQ ID NO: 36 (Tyr Tyr Glu Glu Glu Glu), and SEQ ID NO: 37 (Cys Ala Ser Ser Asn Glu Gly Gln Gly Trp Glu Ala Glu Ala Phe Phe).
   24. A method of predicting whether a patient being diagnosed with a solid tumor will be responsive to treatment of this tumor, wherein cells of the tumor express human melanoma associated antigen 1 (MAGE-A1), the method comprising determining in a tumor cell sample obtained from the patient the fraction of cells that express MAGE-A1, wherein a patient is selected for treatment or determined to be responsive to treatment if a fraction of at least 30% of the cells of the tumor sample are found to express MAGE-A1.
   25. The method of item 24, wherein the patient has been diagnosed with a genotype and/or advanced-Stage metastatic solid tumor that express MAGE-A1.
   26. The method of item 24 or 25, wherein the tumor cell sample from is obtained from a tumor selected from the group consisting of melanoma, lung cancer, esophageal cancer, gastric cancer, breast cancer, ovarian cancer, mesothelioma cancer, bladder cancer, anal cancer, chondrosarcoma cancer, osteosarcoma cancer, sarcoma cancer, adenoma cancer, primitive neuroectodermal cancer (primitive neuroectodermal tumor (PNET), and combinations thereof.
   27. The method of item 26, wherein, the lung cancer is selected from the group consisting of non-small cell lung cancer (NSCLC), including squamous cell carcinoma of the lung, adenocarcinoma of the lung, large cell carcinoma of the lung and other histologic types of NSCLC) and small cell lung cancer.
   28. The method of item 26 or 27, wherein the breast cancer is selected from the group consisting of ductal breast cancer, ductal-invasive breast cancer, invasive breast cancer, tubular breast cancer, medullary breast cancer and combinations thereof.
   29. The method of any one of items 26 to 28, wherein the gastric cancer is gastric adenocarcinoma cancer.
   30. The method of any one of items 26 to 29, wherein the sarcoma cancer is selected from the group consisting of chondrosarcoma cancer, osteosarcoma cancer and combinations thereof.
   31. The method of any one of items 26 to 30, wherein the cancer is selected from the group consisting of gastric adenocarcinoma, pancreatic adenocarcinoma and combinations thereof.
   32. The method of any one of items 24 to 31, wherein the expression of MAGE-A1 in the tumor cell sample is determined by an immunohistochemistry method.
   33. The method of item 32, wherein the immunohistochemistry method is immunostaining.
   34. The method of item 33, wherein the tumor cell sample is embedded in paraffine prior to immunostaining.
   35. The method of item 34, wherein the immunostaining is carried out with an antibody molecule that specifically binds MAGE-A1 or with a proteinaceous binding molecule with antibody-like binding properties that specifically binds MAGE-A1.
   36. The method of item 35, wherein the antibody molecule specifically that binds MAGE-A1 is selected from the group consisting of a polyclonal antibody, a monoclonal antibody, a divalent antibody fragment, and a monovalent antibody fragment.
   37. The method of item 36, wherein the antibody molecule is the monoclonal IgG1 mouse antibody MA454 or a fragment of the antibody MA454.
   38. The method of item 35, wherein the proteinaceous binding molecule with antibody-like binding properties that specifically binds MAGE-A1 is selected from the group consisting of an aptamer, a mutein based on a polypeptide of the lipocalin family, a glubody, a protein based on the ankyrin scaffold, a protein based on the crystalline scaffold, an adnectin, and an avimer.
   39. The method of any one of items 24 to 38, wherein a patient is selected for treatment if a fraction of at least about 50% of the cells, of least about 60% of the cells, of least about 65%, least about 70% of the cells, of at least 75% of the cells, of at least 80% of the cells, of at least about 90% of the cells, of at least about 95% of the cells, of at least about 96% of the cells, of at least about 97% of the cells, of at least about 98% of the cells, of at least about 99% of the cells or 100% of the cells of the tumor sample are found to express MAGE-A1.
   40. The method of item 39, wherein if at least tumor cell samples are obtained from different parts of the tumor and at least one these tumor cell samples is found to express MAGE-A1 in a fraction of at least 30% of the cells of the tumor sample, the patient is selected for treatment.
   41. The method of item 39 or 40, wherein a patient is selected for treatment if a fraction of at least about 50% of the cells, of least about 60% of the cells, of least about 65%, least about 70% of the cells, of at least about 75% of the cells, of at least 80% of the cells, of at least about 95% of the cells, of at least about 96% of the cells, of at least about 97% of the cells, of at least about 98% of the cells, of at least about 99% of the cells or 100 of the cells of the tumor sample are found to express MAGE-A1.
   42. The method of any one of the forgoing items 24 to 41, wherein the treatment is immunotherapy.
   43. The method of item 42, wherein the immunotherapy is by administration of autologous autologous patient-derived T cells.
   44. The method of item 43, wherein the T cells are CD8+ T cells or CD4+ T cells.
   45. The method of item 43 or 44, wherein the autologous T cells express a (recombinant) T cell receptor that specifically binds MAGE A1.
   46. The method of item 45, wherein the T-cell receptor comprises
an alpha chain comprising the CDR sequences shown in SEQ ID NO: 2 (Ile Phe Ser Asn Met Asp Met), SEQ ID NO: 3 (Ile Phe Ser Asn Met Asp Met) and SEQ ID NO: 4 (Cys Ala Glu Ser Ile Gly Ser Asn Ser Gly Tyr Ala Leu Asn Phe); and a beta chain comprising the CDR sequences shown in SEQ ID NO: 5 (Met Asp His Glu Asn), SEQ ID NO: 6 (Ser Tyr Asp Val Lys Met), and SEQ ID NO: 7 (Cys Ala Ser Arg Gly Leu Ala Gly Tyr Glu Gln Tyr Phe);
an alpha chain comprising the CDR sequences shown in SEQ ID NO: 8 (Asp Ser Ala Ser Asn Tyr), SEQ ID NO: 9 (Ile Arg Ser Asn Val Gly Glu), and SEQ ID NO: 10 (Cys Ala Ala Arg Pro Asn Ser Gly Asn Thr Pro Leu Val Phe); and a beta chain comprising the CDR sequences shown in SEQ ID NO: 11 (Ser Gln Val Thr Met), SEQ ID NO: 12 (Ala Asn Gln Gly Ser Glu Ala), and 13 (Cys Ser Val Glu Gln Asp Thr Asn Thr Gly Glu Leu Phe Phe);
an alpha chain comprising the CDR sequences shown in SEQ ID NO: 14 (Asn Ser Ala Phe Gln Tyr), SEQ ID NO: 15 (Thr Tyr Ser Ser Gly Asn), and SEQ ID NO: 16 (Cys Ala Met Ser Asp Thr Gly Asn Gln Phe Tyr Phe); and a beta chain comprising the CDR sequences shown in SEQ ID NO: 17 (Pro Arg His Asp Thr), SEQ ID NO: 18 (Phe Tyr Glu Lys Met Gln), and SEQ ID NO: 19 (Cys Ala Ser Ser Phe Arg Gly Gly Gly Ala Asn Val Leu Thr Phe);
an alpha chain comprising the CDR sequences shown in SEQ ID NO: 20 (Thr Ile Ser Gly Thr Asp Tyr), SEQ ID NO: 21 (Gly) and SEQ ID NO: 22 (Cys Ile Leu Phe Asn Phe Asn Lys Phe Tyr Phe); and a beta chain comprising the CDR sequences shown in SEQ ID NO: 23 (Leu Asn His Asn Val), SEQ ID NO: 24 (Tyr Tyr Asp Lys Asp Phe), and SEQ ID NO: 25 (Cys Ala Thr Ser Ser Gly Glu Thr Asn Glu Lys Leu Phe Phe);
an alpha chain comprising the CDR sequences shown in SEQ ID NO: 26 (Asp Ser Ala Ser Asn Tyr), SEQ ID NO: 27 (Ile Arg Ser) and SEQ ID NO: 28 (Cys Ala Ala Ser Pro Thr Gly Gly Tyr Asn Lys Leu Ile Phe); and a beta chain comprising the CDR sequences shown in SEQ ID NO: 29 (Met Asn His Glu Tyr), SEQ ID NO: 30 (Ser Val Gly Ala Gly Ile), and SEQ ID NO: 31 (Cys Ala Ser Ser Leu Gly Gly Ala Ser Gln Glu Gln Tyr Phe); or
an alpha chain comprising the CDR sequences shown in SEQ ID NO: 32 (Thr Ser Glu Ser Asn Tyr Tyr), SEQ ID NO: 33 (Gln Glu Ala Tyr) and SEQ ID NO: 34 (Cys Ala Phe Gly Tyr Ser Gly Gly Gly Ala Asp Gly Leu Thr Phe); and a beta chain comprising the CDR sequences shown in SEQ ID NO: 35 (Ser Gly His Asp Thr), SEQ ID NO: 36 (Tyr Tyr Glu Glu Glu Glu), and SEQ ID NO: 37 (Cys Ala Ser Ser Asn Glu Gly Gln Gly Trp Glu Ala Glu Ala Phe Phe).
   47. A method of treating a patient being diagnosed with a solid tumor, wherein cells of the tumor express human melanoma associated antigen 1 (MAGE-A1), wherein a patient is selected for treatment if a fraction of at least 30% of the cells of a tumor cell sample obtained from the patient are found to express MAGE-A1, wherein the method comprises administering to the patient a therapeutically effective amount of an adoptive cell therapy agent or an agent specifically binding MAGE-A1.
   48. A method of treating a patient having a solid tumor, wherein a fraction of at least 30% of cells of a sample of the tumor obtained from the patient has been determined to express MAGE-A1, the method comprising administering to the patient a therapeutically effective amount of an adoptive cell therapy agent or an agent specifically binding MAGE-A1.
   49. A method of treating a patient having a solid tumor, the method comprising:
a) determining that at least 30% of cells of a sample of the tumor obtained from the patient express MAGE-A1; and
b) administering to the patient a therapeutically effective amount of an adoptive cell therapy agent or an agent specifically binding MAGE-A1.
   50. The method of any one of items 47 to 49, wherein the adoptive cell therapy agent is selected from the group consisting of a chimeric antigen receptor T-cell (CAR T-cell), a genetically modified T-cell and a genetically modified NK cells each of expresses a (recombinant) T cell receptor that specifically binds MAGE A1.
   51. The method of item 50, wherein the T-cell receptor comprises
an alpha chain comprising the CDR sequences shown in SEQ ID NO: 2 (Ile Phe Ser Asn Met Asp Met), SEQ ID NO: 3 (Ile Phe Ser Asn Met Asp Met) and SEQ ID NO: 4 (Cys Ala Glu Ser Ile Gly Ser Asn Ser Gly Tyr Ala Leu Asn Phe); and a beta chain comprising the CDR sequences shown in SEQ ID NO: 5 (Met Asp His Glu Asn), SEQ ID NO: 6 (Ser Tyr Asp Val Lys Met), and SEQ ID NO: 7 (Cys Ala Ser Arg Gly Leu Ala Gly Tyr Glu Gln Tyr Phe);
an alpha chain comprising the CDR sequences shown in SEQ ID NO: 8 (Asp Ser Ala Ser Asn Tyr), SEQ ID NO: 9 (Ile Arg Ser Asn Val Gly Glu), and SEQ ID NO: 10 (Cys Ala Ala Arg Pro Asn Ser Gly Asn Thr Pro Leu Val Phe); and a beta chain comprising the CDR sequences shown in SEQ ID NO: 11 (Ser Gln Val Thr Met), SEQ ID NO: 12 (Ala Asn Gln Gly Ser Glu Ala), and 13 (Cys Ser Val Glu Gln Asp Thr Asn Thr Gly Glu Leu Phe Phe);
an alpha chain comprising the CDR sequences shown in SEQ ID NO: 14 (Asn Ser Ala Phe Gln Tyr), SEQ ID NO: 15 (Thr Tyr Ser Ser Gly Asn), and SEQ ID NO: 16 (Cys Ala Met Ser Asp Thr Gly Asn Gln Phe Tyr Phe); and a beta chain comprising the CDR sequences shown in SEQ ID NO: 17 (Pro Arg His Asp Thr), SEQ ID NO: 18 (Phe Tyr Glu Lys Met Gln), and SEQ ID NO: 19 (Cys Ala Ser Ser Phe Arg Gly Gly Gly Ala Asn Val Leu Thr Phe);
an alpha chain comprising the CDR sequences shown in SEQ ID NO: 20 (Thr Ile Ser Gly Thr Asp Tyr), SEQ ID NO: 21 (Gly) and SEQ ID NO: 22 (Cys Ile Leu Phe Asn Phe Asn Lys Phe Tyr Phe); and a beta chain comprising the CDR sequences shown in SEQ ID NO: 23 (Leu Asn His Asn Val), SEQ ID NO: 24 (Tyr Tyr Asp Lys Asp Phe), and SEQ ID NO: 25 (Cys Ala Thr Ser Ser Gly Glu Thr Asn Glu Lys Leu Phe Phe);
an alpha chain comprising the CDR sequences shown in SEQ ID NO: 26 (Asp Ser Ala Ser Asn Tyr), SEQ ID NO: 27 (Ile Arg Ser) and SEQ ID NO: 28 (Cys Ala Ala Ser Pro Thr Gly Gly Tyr Asn Lys Leu Ile Phe); and a beta chain comprising the CDR sequences shown in SEQ ID NO: 29 (Met Asn His Glu Tyr), SEQ ID NO: 30 (Ser Val Gly Ala Gly Ile), and SEQ ID NO: 31 (Cys Ala Ser Ser Leu Gly Gly Ala Ser Gln Glu Gln Tyr Phe); or
an alpha chain comprising the CDR sequences shown in SEQ ID NO: 32 (Thr Ser Glu Ser Asn Tyr Tyr), SEQ ID NO: 33 (Gln Glu Ala Tyr) and SEQ ID NO: 34 (Cys Ala Phe Gly Tyr Ser Gly Gly Gly Ala Asp Gly Leu Thr Phe); and a beta chain comprising the CDR sequences shown in SEQ ID NO: 35 (Ser Gly His Asp Thr), SEQ ID NO: 36 (Tyr Tyr Glu Glu Glu Glu), and SEQ ID NO: 37 (Cys Ala Ser Ser Asn Glu Gly Gln Gly Trp Glu Ala Glu Ala Phe Phe).
   52. The method of item 50 or 51, wherein the T-cells are autologous patient-derived T cells or allogenic T cells.
   53. The method of item 52, wherein the T cells are selected from the group consisting of CD8+ T cells, CD4+ T cells, a combination thereof and T cells of other phenotypes.
   54. The method of any one of items 50 to 53, wherein the dosage of the T cells administered to the patient, defined as the total number of T cells, is from about 0.5×10⁷ T cells to about 1×10¹⁰ T cells.
   55. The method of item 54, wherein the dosage of the T cells administered to the patient, defined as the total number of T cells, is about 0.75×1×10⁸ T cells, about 1×10⁸ cells, about 1×10⁹ T cells, about 3×10⁹ T cells, about 4×10⁹ T cells, about 5×10⁹ T cells, about 6×10⁹ T cells, about 7×10⁹ T cells, about 8×10⁹ T cells or about 9×10⁹ T cells.
   56. The method of item 54 or 55, wherein the dosage of the T cells administered to the patient, defined as the total number of T cells, is in the range of about 1×10⁹ T cells, to about 9×10⁹ T cells or in the rage of about 3×10⁹ T cells to about 9×10⁹ T cells.
   57. The method of any one of items 50 to 56, wherein the T cells are as administered as a single dose.
   58. The method of any one of items 47 to 57, wherein the adoptive cell therapy agent or the agent specifically binding MAGE-A1 is administered by infusion.
   59. The method of any one of items 47 to 49, wherein the agent specifically binding MAGE-A1 is a drug-ligand conjugate, wherein the ligand is an antibody molecule that specifically binds MAGE-A1 or a proteinaceous binding molecule with antibody-like binding properties that specifically binds MAGE-A1.
   60. The method of item 59, wherein the drug is cytotoxic or cytostatic molecule.
   61. The method of any one of items 47 to 60, wherein the patient has been determined to have a HLA-A*02 genotype.
   62. The method of item 61, wherein the HLA-A*02 genotype is the HLA-A*02:01 genotype.
   63. The method of any one of items 47 and 50 to 62, wherein a patient is selected for treatment if a fraction of at least about 50% of the cells, of least about 60% of the cells, of least about 65%, least about 70% of the cells, of at least about 75% of the cells of at least about 80% of the cells, of at least about 90% of the cells, of at least about 95% of the cells, of at least about 96% of the cells, of at least about 97% of the cells, of at least about 98% of the cells, of at least about 99% of the cells or 100% of the cells of the tumor sample are found to express MAGE-A1.
   64. The method of any one of items 48 to 62, wherein at least about 50% of the cells of the sample, at least about 60% of the cells of the sample, at least about 65% of the cells of the sample, at least about 70% of the cells of the sample, or at least about 75% of the cells of the sample, at least about 80% of the cells, of at least about 90% of the cells, of at least about 95% of the cells, of at least about 96% of the cells, of at least about 97% of the cells, of at least about 98% of the cells, of at least about 99% of the cells or 100% of the cells of the sample are determined to express MAGE-A1.
   65. The method of any one of items 47 and 50 to 62, wherein selecting a patient for treatment comprises determining the fraction of tumor cells expressing MAGE-A1 in the tumor cell sample by an immunohistochemistry method.
   66. The method of any one of items 48 to 62, wherein expression of MAGE-A1 in the cells of the sample is assessed by way of an immunohistochemistry method.
   67. The method of item 65 or 66, wherein the immunohistochemistry method is immunostaining.
   68. The method of item 67, wherein the tumor cell sample is embedded in paraffine prior to immunostaining.
   69. The method of any of items 67 or 68, wherein the immunostaining is carried out with an antibody molecule that specifically binds MAGE-A1 or with a proteinaceous binding molecule with antibody-like binding properties that specifically binds MAGE-A1.
   70. The method of item 69, wherein the antibody molecule specifically that binds MAGE-A1 is selected from the group consisting of a polyclonal antibody, a monoclonal antibody, a divalent antibody fragment, and a monovalent antibody fragment.
   71. The method of item 70, wherein the antibody molecule is the monoclonal IgG1 mouse antibody MA454 or a fragment of the antibody MA454.
   72. A pharmaceutical composition comprising T cells expressing a T cell receptor that specifically binds MAGE A1, wherein the T-cell receptor comprises
an alpha chain comprising the CDR sequences shown in SEQ ID NO: 2 (Ile Phe Ser Asn Met Asp Met), SEQ ID NO: 3 (Ile Phe Ser Asn Met Asp Met) and SEQ ID NO: 4 (Cys Ala Glu Ser Ile Gly Ser Asn Ser Gly Tyr Ala Leu Asn Phe); and a beta chain comprising the CDR sequences shown in SEQ ID NO: 5 (Met Asp His Glu Asn), SEQ ID NO: 6 (Ser Tyr Asp Val Lys Met), and SEQ ID NO: 7 (Cys Ala Ser Arg Gly Leu Ala Gly Tyr Glu Gln Tyr Phe);
an alpha chain comprising the CDR sequences shown in SEQ ID NO: 8 (Asp Ser Ala Ser Asn Tyr), SEQ ID NO: 9 (Ile Arg Ser Asn Val Gly Glu), and SEQ ID NO: 10 (Cys Ala Ala Arg Pro Asn Ser Gly Asn Thr Pro Leu Val Phe); and a beta chain comprising the CDR sequences shown in SEQ ID NO: 11 (Ser Gln Val Thr Met), SEQ ID NO: 12 (Ala Asn Gln Gly Ser Glu Ala), and 13 (Cys Ser Val Glu Gln Asp Thr Asn Thr Gly Glu Leu Phe Phe); or
an alpha chain comprising the CDR sequences shown in SEQ ID NO: 14 (Asn Ser Ala Phe Gln Tyr), SEQ ID NO: 15 (Thr Tyr Ser Ser Gly Asn), and SEQ ID NO: 16 (Cys Ala Met Ser Asp Thr Gly Asn Gln Phe Tyr Phe); and a beta chain comprising the CDR sequences shown in SEQ ID NO: 17 (Pro Arg His Asp Thr), SEQ ID NO: 18 (Phe Tyr Glu Lys Met Gln), and SEQ ID NO: 19 (Cys Ala Ser Ser Phe Arg Gly Gly Gly Ala Asn Val Leu Thr Phe);
   and wherein the total number of T cells comprised in the composition is from about 0.5×10⁷ T cells to about 1×10¹⁰ T cells.
   73. The pharmaceutical composition of item 72, wherein the total number of T cells comprised in the composition is about 0.75×1×10⁸ T cells, about 1×10⁸ cells, about 1×10⁹ T cells, about 3×10⁹ T cells, about 4×10⁹ T cells, about 5×10⁹ T cells, about 6×10⁹ T cells, about 7×10⁹ T cells, about 8×10⁹ T cells or about 9×10⁹ T cells.
   74. The pharmaceutical composition of item 72 or 73, further comprising a pharmaceutically acceptable carrier.
   75. The pharmaceutical composition of item 74, wherein the pharmaceutically acceptable carrier is a physiological saline solution.
   76. The use of the monoclonal IgG1 mouse antibody MA454 or a fragment of the antibody MA454 for selecting a patient for treatment of a solid tumor, wherein cells of the tumor express the human melanoma associated antigen 1 (MAGE-A1) (UniProtKB accession number P43355 (MAGA1_HUMAN).
   77. The use of item 76, the use comprising determining in a tumor cell sample obtained from the patient the fraction of cells that express MAGE-A1, wherein a patient is selected for treatment if a fraction of at least 30% of the cells of the tumor sample are found to express MAGE-A1.
   78. The use of the monoclonal IgG1 mouse antibody MA454 or a fragment of the antibody MA454 for predicting whether a patient being diagnosed with a solid tumor will be responsive to treatment of this tumor, wherein cells of the tumor express human melanoma associated antigen 1 (MAGE-A1) (UniProtKB accession number P43355 (MAGA1_HUMAN).
   79. The use of item 78, the use comprising determining in a tumor cell sample obtained from the patient the fraction of cells that express MAGE-A1, wherein a patient is selected for treatment if a fraction of at least 30% of the cells of the tumor sample are found to express MAGE-A1.
   80. An adoptive cell therapy agent specifically binding MAGE-A1 or an agent specifically binding MAGE-A1 for use in treating a patient being diagnosed with a solid tumor, wherein cells of the tumor express human melanoma associated antigen 1 (MAGE-A1), wherein a patient is selected for treatment if a fraction of at least 30% of the cells of a tumor sample obtained from the patient are found to express MAGE-A1.
   81. The adoptive cell therapy agent specifically binding MAGE-A1 or an agent specifically binding MAGE-A1 for the use of item 80, wherein the method comprises administering to the patient a therapeutically effective amount of an adoptive cell therapy agent specifically binding MAGE-A1 or an agent specifically binding MAGE-A1.
   82. The adoptive cell therapy agent specifically binding MAGE-A1 or an agent specifically binding MAGE-A1 for the use of item 80 or 81, wherein the adoptive cell therapy agent is a chimeric antigen receptor T-cell (CAR T-cell) or a genetically modified T-cell that expresses a (recombinant) T cell receptor that specifically binds MAGE A1.
   83. The adoptive cell therapy agent for the use of item 82, wherein the T-cell receptor comprises
an alpha chain comprising the CDR sequences shown in SEQ ID NO: 2 (Ile Phe Ser Asn Met Asp Met), SEQ ID NO: 3 (Ile Phe Ser Asn Met Asp Met) and SEQ ID NO: 4 (Cys Ala Glu Ser Ile Gly Ser Asn Ser Gly Tyr Ala Leu Asn Phe); and a beta chain comprising the CDR sequences shown in SEQ ID NO: 5 (Met Asp His Glu Asn), SEQ ID NO: 6 (Ser Tyr Asp Val Lys Met), and SEQ ID NO: 7 (Cys Ala Ser Arg Gly Leu Ala Gly Tyr Glu Gln Tyr Phe);
an alpha chain comprising the CDR sequences shown in SEQ ID NO: 8 (Asp Ser Ala Ser Asn Tyr), SEQ ID NO: 9 (Ile Arg Ser Asn Val Gly Glu), and SEQ ID NO: 10 (Cys Ala Ala Arg Pro Asn Ser Gly Asn Thr Pro Leu Val Phe); and a beta chain comprising the CDR sequences shown in SEQ ID NO: 11 (Ser Gln Val Thr Met), SEQ ID NO: 12 (Ala Asn Gln Gly Ser Glu Ala), and 13 (Cys Ser Val Glu Gln Asp Thr Asn Thr Gly Glu Leu Phe Phe);
an alpha chain comprising the CDR sequences shown in SEQ ID NO: 14 (Asn Ser Ala Phe Gln Tyr), SEQ ID NO: 15 (Thr Tyr Ser Ser Gly Asn), and SEQ ID NO: 16 (Cys Ala Met Ser Asp Thr Gly Asn Gln Phe Tyr Phe); and a beta chain comprising the CDR sequences shown in SEQ ID NO: 17 (Pro Arg His Asp Thr), SEQ ID NO: 18 (Phe Tyr Glu Lys Met Gln), and SEQ ID NO: 19 (Cys Ala Ser Ser Phe Arg Gly Gly Gly Ala Asn Val Leu Thr Phe);
an alpha chain comprising the CDR sequences shown in SEQ ID NO: 20 (Thr Ile Ser Gly Thr Asp Tyr), SEQ ID NO: 21 (Gly) and SEQ ID NO: 22 (Cys Ile Leu Phe Asn Phe Asn Lys Phe Tyr Phe); and a beta chain comprising the CDR sequences shown in SEQ ID NO: 23 (Leu Asn His Asn Val), SEQ ID NO: 24 (Tyr Tyr Asp Lys Asp Phe), and SEQ ID NO: 25 (Cys Ala Thr Ser Ser Gly Glu Thr Asn Glu Lys Leu Phe Phe);
an alpha chain comprising the CDR sequences shown in SEQ ID NO: 26 (Asp Ser Ala Ser Asn Tyr), SEQ ID NO: 27 (Ile Arg Ser) and SEQ ID NO: 28 (Cys Ala Ala Ser Pro Thr Gly Gly Tyr Asn Lys Leu Ile Phe); and a beta chain comprising the CDR sequences shown in SEQ ID NO: 29 (Met Asn His Glu Tyr), SEQ ID NO: 30 (Ser Val Gly Ala Gly Ile), and SEQ ID NO: 31 (Cys Ala Ser Ser Leu Gly Gly Ala Ser Gln Glu Gln Tyr Phe); or
an alpha chain comprising the CDR sequences shown in SEQ ID NO: 32 (Thr Ser Glu Ser Asn Tyr Tyr), SEQ ID NO: 33 (Gln Glu Ala Tyr) and SEQ ID NO: 34 (Cys Ala Phe Gly Tyr Ser Gly Gly Gly Ala Asp Gly Leu Thr Phe); and a beta chain comprising the CDR sequences shown in SEQ ID NO: 35 (Ser Gly His Asp Thr), SEQ ID NO: 36 (Tyr Tyr Glu Glu Glu Glu), and SEQ ID NO: 37 (Cys Ala Ser Ser Asn Glu Gly Gln Gly Trp Glu Ala Glu Ala Phe Phe).
   84. The adoptive cell therapy agent specifically binding MAGE-A1 or an agent specifically binding MAGE-A1 for the use of any one of items 76 to 72, wherein a patient is selected for treatment if a fraction of at least about 50% of the cells, of least about 60% of the cells, of least about 65%, least about 70% of the cells, of at least about 75% of the cells, of at least 80% of the cells, of at least about 90% of the cells, of at least about 95% of the cells, of at least about 96% of the cells, of at least about 97% of the cells, of at least about 98% of the cells, of at least about 99% of the cells or 100% of the cells of the tumor sample are found to express MAGE-A1.
   85. The adoptive cell therapy agent specifically binding MAGE-A1 or an agent specifically binding MAGE-A1 for the use of item 84, wherein selecting a patient for treatment comprises determining the fraction of tumor cells expressing MAGE-A1 in the tumor cell sample by an immunohistochemistry method.
   86. The adoptive cell therapy agent specifically binding MAGE-A1 or an agent specifically binding MAGE-A1 for the use of item 85, wherein the immunohistochemistry method is immunostaining.
   87. The adoptive cell therapy agent specifically binding MAGE-A1 or an agent specifically binding MAGE-A1 for the use of item 86, wherein the tumor cell sample is embedded in paraffine prior to immunostaining.
   88. The adoptive cell therapy agent specifically binding MAGE-A1 or an agent specifically binding MAGE-A1 for the use of any of items 86 or 87, wherein the immunostaining is carried out with an antibody molecule that specifically binds MAGE-A1 or with a proteinaceous binding molecule with antibody-like binding properties that specifically binds MAGE-A1.
   89. The adoptive cell therapy agent specifically binding MAGE-A1 or an agent specifically binding MAGE-A1 for the use of item 88, wherein the antibody molecule specifically that binds MAGE-A1 is selected from the group consisting of a polyclonal antibody, a monoclonal antibody, a divalent antibody fragment, and a monovalent antibody fragment.
   90. The adoptive cell therapy agent specifically binding MAGE-A1 or an agent specifically binding MAGE-A1 for the use of item 89, wherein the antibody molecule is the monoclonal IgG1 mouse antibody MA454 or a fragment of the antibody MA454.
   91. A diagnostic immunostaining kit for selecting a patient for treatment of a solid tumor, wherein cells of the tumor express the human melanoma associated antigen 1 (MAGE-A1) (e.g. UniProtKB accession number P43355 (MAGA1_HUMAN), wherein the kit comprises
the monoclonal IgG1 mouse antibody MA454 or an antigen binding fragment of the antibody MA454, and
a secondary antibody capable of binding to the monoclonal IgG1 mouse antibody MA454.
   92. The kit of item 91, wherein the secondary antibody comprises an optically detectable label.
   93. The kit of item 92, wherein the optically detectable label is an enzyme catalyzing a chromogenic reaction.
   94. The kit of item 92 or 93, wherein the enzyme is conjugated to a polymer.
   95. The kit of item 94, wherein the polymer is a dextran.
   96. The kit of any one of items 91 to 95, wherein the optically detectable label is conjugated to the secondary antibody.
   97. The kit of any of one of items 91 to 96, wherein the monoclonal IgG1 mouse antibody MA454 or a fragment of the antibody MA454, and the secondary antibody are packaged in individual containers.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention. The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group. Further embodiments of the invention will become apparent from the following claims.

What is claimed is:

1. A method of selecting a patient for treatment of a solid tumor, wherein cells of the tumor express human melanoma associated antigen 1 (MAGE-A1) (e.g., UniProtKB accession number P43355 (MAGA1_HUMAN), the method comprising determining in a tumor cell sample obtained from the patient the fraction of cells that express MAGE-A1, wherein a patient is selected for treatment if a fraction of at least 30% of the cells of the tumor sample are found to express MAGE-A1.

2. The method of claim 1, wherein the patient has been diagnosed with a corresponding HLA genotype and/or advanced-Stage metastatic solid tumor that express MAGE-A1.

3. The method of claim 1, wherein the tumor cell sample from is obtained from a tumor selected from the group consisting of melanoma, lung cancer, esophageal cancer, gastric cancer, breast cancer, ovarian cancer, mesothelioma cancer, bladder cancer, anal cancer, chondrosarcoma cancer, osteosarcoma cancer, sarcoma cancer, adenoma cancer, primitive neuroectodermal cancer (primitive neuroectodermal tumor (PNET), and combinations thereof.

4. The method of claim 3, wherein the lung cancer is selected from the group consisting of non-small cell lung cancer (NSCLC), including squamous cell carcinoma of the lung, adenocarcinoma of the lung, large cell carcinoma of the lung and other histologic types of NSCLC) and small cell lung cancer.

5. The method of claim 1, wherein the solid tumor is being determined as a MAGE-1A homogeneously positive tumor.

6. The method of claim 1, wherein the expression of MAGE-A1 in the tumor cell sample is determined by an immunohistochemistry method.

7. The method of claim 6, wherein the immunohistochemistry method is immunostaining.

8. The method of claim 7, wherein the immunostaining is carried out with an antibody molecule that specifically binds MAGE-A1 or with a proteinaceous binding molecule with antibody-like binding properties that specifically binds MAGE-A1.

9. The method of claim 8, wherein the antibody molecule specifically that binds MAGE-A1 is selected from the group consisting of a polyclonal antibody, a monoclonal antibody, a divalent antibody fragment, and a monovalent antibody fragment.

10. The method of claim 9, wherein the antibody molecule is the monoclonal IgG1 mouse antibody MA454 or a fragment of the antibody MA454.

11. The method of claim 1, wherein a patient is selected for treatment if a fraction of at least about 50% of the cells, of least about 60% of the cells, of least about 65%, least about 70% of the cells, of at least about 75% of the cells, of at least about 80% of the cells, of at least about 90% of the cells, of at least about 95% of the cells, of at least about 96% of the cells, of at least about 97% of the cells, of at least about 98% of the cells, of at least about 99% of the cells or 100% of the cells of the tumor sample are found to express MAGE-A1.

12. The method of claim 11, wherein if at least tumor cell samples are obtained from different parts of the tumor and at least one these tumor cell samples is found to express MAGE-A1 in a fraction of at least 30% of the cells of the tumor sample, the patient is selected for treatment.

13. The method of claim 1, wherein the treatment is immunotherapy.

14. The method of claim 13, wherein the immunotherapy is by administration cells selected from the group consisting of autologous patient-derived T cells, allogeneic T cells and NK cells.

15. The method of claim 14, wherein the autologous T cells express a recombinant T cell receptor that specifically binds MAGE A1.

16. The method of claim 15, wherein the T-cell receptor comprises
an alpha chain comprising the CDR sequences shown in SEQ ID NO: 2 (Ile Phe Ser Asn Met Asp Met), SEQ ID NO: 3 (Ile Phe Ser Asn Met Asp Met) and SEQ ID NO: 4 (Cys Ala Glu Ser Ile Gly Ser Asn Ser Gly Tyr Ala Leu Asn Phe); and a beta chain comprising the CDR sequences shown in SEQ ID NO: 5 (Met Asp His Glu Asn), SEQ ID NO: 6 (Ser Tyr Asp Val Lys Met), and SEQ ID NO: 7 (Cys Ala Ser Arg Gly Leu Ala Gly Tyr Glu Gln Tyr Phe);
an alpha chain comprising the CDR sequences shown in SEQ ID NO: 8 (Asp Ser Ala Ser Asn Tyr), SEQ ID NO: 9 (Ile Arg Ser Asn Val Gly Glu), and SEQ ID NO: 10 (Cys Ala Ala Arg Pro Asn Ser Gly Asn Thr Pro Leu Val Phe); and a beta chain comprising the CDR sequences shown in SEQ ID NO: 11 (Ser Gln Val Thr Met), SEQ ID NO: 12 (Ala Asn Gln Gly Ser Glu Ala), and 13 (Cys Ser Val Glu Gln Asp Thr Asn Thr Gly Glu Leu Phe Phe);
an alpha chain comprising the CDR sequences shown in SEQ ID NO: 14 (Asn Ser Ala Phe Gln Tyr), SEQ ID NO: 15 (Thr Tyr Ser Ser Gly Asn), and SEQ ID NO: 16 (Cys Ala Met Ser Asp Thr Gly Asn Gln Phe Tyr Phe); and a beta chain comprising the CDR sequences shown in SEQ ID NO: 17 (Pro Arg His Asp Thr), SEQ ID NO: 18 (Phe Tyr Glu Lys Met Gln), and SEQ ID NO: 19 (Cys Ala Ser Ser Phe Arg Gly Gly Gly Ala Asn Val Leu Thr Phe);
an alpha chain comprising the CDR sequences shown in SEQ ID NO: 20 (Thr Ile Ser Gly Thr Asp Tyr), SEQ ID NO: 21 (Gly) and SEQ ID NO: 22 (Cys Ile Leu Phe Asn Phe Asn Lys Phe Tyr Phe); and a beta chain comprising the CDR sequences shown in SEQ ID NO: 23 (Leu Asn His Asn Val), SEQ ID NO: 24 (Tyr Tyr Asp Lys Asp Phe), and SEQ ID NO: 25 (Cys Ala Thr Ser Ser Gly Glu Thr Asn Glu Lys Leu Phe Phe);
an alpha chain comprising the CDR sequences shown in SEQ ID NO: 26 (Asp Ser Ala Ser Asn Tyr), SEQ ID NO: 27 (Ile Arg Ser) and SEQ ID NO: 28 (Cys Ala Ala Ser Pro Thr Gly Gly Tyr Asn Lys Leu Ile Phe); and a beta chain comprising the CDR sequences shown in SEQ ID NO: 29 (Met Asn His Glu Tyr), SEQ ID NO: 30 (Ser Val Gly Ala Gly Ile), and SEQ ID NO: 31 (Cys Ala Ser Ser Leu Gly Gly Ala Ser Gln Glu Gln Tyr Phe); or
an alpha chain comprising the CDR sequences shown in SEQ ID NO: 32 (Thr Ser Glu Ser Asn Tyr Tyr), SEQ ID NO: 33 (Gln Glu Ala Tyr) and SEQ ID NO: 34 (Cys Ala Phe Gly Tyr Ser Gly Gly Gly Ala Asp Gly Leu Thr Phe); and a beta chain comprising the CDR sequences shown in SEQ ID NO: 35 (Ser Gly His Asp Thr), SEQ ID NO: 36 (Tyr Tyr Glu Glu Glu Glu), and SEQ ID NO: 37 (Cys Ala Ser Ser Asn Glu Gly Gln Gly Trp Glu Ala Glu Ala Phe Phe).

17. A method of predicting whether a patient being diagnosed with a solid tumor will be responsive to treatment of this tumor, wherein cells of the tumor express human melanoma associated antigen 1 (MAGE-A1), the method comprising determining in a tumor cell sample obtained from the patient the fraction of cells that express MAGE-A1, wherein a patient is selected for treatment if a fraction of at least 30% of the cells of the tumor sample are found to express MAGE-A1.

18. A method of treating a patient being diagnosed with a solid tumor, wherein cells of the tumor express human melanoma associated antigen 1 (MAGE-A1), wherein a patient is selected for treatment if a fraction of at least 30% of the cells of a tumor cell sample obtained from the patient are found to express MAGE-A1, wherein the method comprises administering to the patient a therapeutically effective amount of an adoptive cell therapy agent or an agent specifically binding MAGE-A1.

19. A method of treating a patient having a solid tumor, wherein a fraction of at least 30% of cells of a sample of the tumor obtained from the patient has been determined to express MAGE-A1, the method comprising administering to the patient a therapeutically effective amount of an adoptive cell therapy agent or an agent specifically binding MAGE-A1.

20. A method of treating a patient having a solid tumor, the method comprising:
a) determining that at least 30% of cells of a sample of the tumor obtained from the patient express MAGE-A1; and
b) administering to the patient a therapeutically effective amount of an adoptive cell therapy agent or an agent specifically binding MAGE-A1.

21. The method of claim 20, wherein the adoptive cell therapy agent is selected from the group consisting of a chimeric antigen receptor T-cell (CAR T-cell), a genetically modified T-cell and a genetically modified NK cells each of expresses a (recombinant) T cell receptor that specifically binds MAGE A1.

22. The method of claim 21, wherein the T-cell receptor comprises
an alpha chain comprising the CDR sequences shown in SEQ ID NO: 2 (Ile Phe Ser Asn Met Asp Met), SEQ ID NO: 3 (Ile Phe Ser Asn Met Asp Met) and SEQ ID NO: 4 (Cys Ala Glu Ser Ile Gly Ser Asn Ser Gly Tyr Ala Leu Asn Phe); and a beta chain comprising the CDR sequences shown in SEQ ID NO: 5 (Met Asp His Glu Asn), SEQ ID NO: 6 (Ser Tyr Asp Val Lys Met), and SEQ ID NO: 7 (Cys Ala Ser Arg Gly Leu Ala Gly Tyr Glu Gln Tyr Phe);
an alpha chain comprising the CDR sequences shown in SEQ ID NO: 8 (Asp Ser Ala Ser Asn Tyr), SEQ ID NO: 9 (Ile Arg Ser Asn Val Gly Glu), and SEQ ID NO: 10 (Cys Ala Ala Arg Pro Asn Ser Gly Asn Thr Pro Leu Val Phe); and a beta chain comprising the CDR sequences shown in SEQ ID NO: 11 (Ser Gln Val Thr Met), SEQ ID NO: 12 (Ala Asn Gln Gly Ser Glu Ala), and 13 (Cys Ser Val Glu Gln Asp Thr Asn Thr Gly Glu Leu Phe Phe);
an alpha chain comprising the CDR sequences shown in SEQ ID NO: 14 (Asn Ser Ala Phe Gln Tyr), SEQ ID NO: 15 (Thr Tyr Ser Ser Gly Asn), and SEQ ID NO: 16 (Cys Ala Met Ser Asp Thr Gly Asn Gln Phe Tyr Phe); and a beta chain comprising the CDR sequences shown in SEQ ID NO: 17 (Pro Arg His Asp Thr), SEQ ID NO: 18 (Phe Tyr Glu Lys Met Gln), and SEQ ID NO: 19 (Cys Ala Ser Ser Phe Arg Gly Gly Gly Ala Asn Val Leu Thr Phe);
an alpha chain comprising the CDR sequences shown in SEQ ID NO: 20 (Thr Ile Ser Gly Thr Asp Tyr), SEQ ID NO: 21 (Gly) and SEQ ID NO: 22 (Cys Ile Leu Phe Asn Phe Asn Lys Phe Tyr Phe); and a beta chain comprising the CDR sequences shown in SEQ ID NO: 23 (Leu Asn His Asn Val), SEQ ID NO: 24 (Tyr Tyr Asp Lys Asp Phe), and SEQ ID NO: 25 (Cys Ala Thr Ser Ser Gly Glu Thr Asn Glu Lys Leu Phe Phe);
an alpha chain comprising the CDR sequences shown in SEQ ID NO: 26 (Asp Ser Ala Ser Asn Tyr), SEQ ID NO: 27 (Ile Arg Ser) and SEQ ID NO: 28 (Cys Ala Ala Ser Pro Thr Gly Gly Tyr Asn Lys Leu Ile Phe); and a beta chain comprising the CDR sequences shown in SEQ ID NO: 29 (Met Asn His Glu Tyr), SEQ ID NO: 30 (Ser Val Gly Ala Gly Ile), and SEQ ID NO: 31 (Cys Ala Ser Ser Leu Gly Gly Ala Ser Gln Glu Gln Tyr Phe); or
an alpha chain comprising the CDR sequences shown in SEQ ID NO: 32 (Thr Ser Glu Ser Asn Tyr Tyr), SEQ ID NO: 33 (Gln Glu Ala Tyr) and SEQ ID NO: 34 (Cys Ala Phe Gly Tyr Ser Gly Gly Gly Ala Asp Gly Leu Thr Phe); and a beta chain comprising the CDR sequences shown in SEQ ID NO: 35 (Ser Gly His Asp Thr), SEQ ID NO: 36 (Tyr Tyr Glu Glu Glu Glu), and SEQ ID NO: 37 (Cys Ala Ser Ser Asn Glu Gly Gln Gly Trp Glu Ala Glu Ala Phe Phe).

23. The method of claim 22, wherein the T-cells are autologous patient-derived T cells or allogenic T cells.

24. The method of claim 23, wherein the dosage of the T cells administered to the patient, defined as the total number of T cells, is from about 0.5×10⁷ T cells to about 1×10¹⁰ T cells.

25. The method of claim 24, wherein the dosage of the T cells administered to the patient, defined as the total number of T cells, is about 0.75×1×10⁸ T cells, about 1×10⁸ cells, about 1×10⁹ T cells, about 3×10⁹ T cells, about 4×10⁹ T cells, about 5×10⁹ T cells, about 6×10⁹ T cells, about 7×10⁹ T cells, about 8×10⁹ T cells or about 9×10⁹T cells.

26. The method of claim 20, wherein the patient has been determined to have a HLA-A*02 genotype.

27. The method of claim 20, wherein selecting a patient for treatment comprises determining the fraction of tumor cells expressing MAGE-A1 in the tumor cell sample by an immunohistochemistry method.

28. A pharmaceutical composition comprising T cells expressing a T cell receptor that specifically binds MAGE A1, wherein the T-cell receptor comprises
an alpha chain comprising the CDR sequences shown in SEQ ID NO: 2 (Ile Phe Ser Asn Met Asp Met), SEQ ID NO: 3 (Ile Phe Ser Asn Met Asp Met) and SEQ ID NO: 4 (Cys Ala Glu Ser Ile Gly Ser Asn Ser Gly Tyr Ala Leu Asn Phe); and a beta chain comprising the CDR sequences shown in SEQ ID NO: 5 (Met Asp His Glu Asn), SEQ ID NO: 6 (Ser Tyr Asp Val Lys Met), and SEQ ID NO: 7 (Cys Ala Ser Arg Gly Leu Ala Gly Tyr Glu Gln Tyr Phe);
an alpha chain comprising the CDR sequences shown in SEQ ID NO: 8 (Asp Ser Ala Ser Asn Tyr), SEQ ID NO: 9 (Ile Arg Ser Asn Val Gly Glu), and SEQ ID NO: 10 (Cys Ala Ala Arg Pro Asn Ser Gly Asn Thr Pro Leu Val Phe); and a beta chain comprising the CDR sequences shown in SEQ ID NO: 11 (Ser Gln Val Thr Met), SEQ ID NO: 12 (Ala Asn Gln Gly Ser Glu Ala), and 13 (Cys Ser Val Glu Gln Asp Thr Asn Thr Gly Glu Leu Phe Phe); or
an alpha chain comprising the CDR sequences shown in SEQ ID NO: 14 (Asn Ser Ala Phe Gln Tyr), SEQ ID NO: 15 (Thr Tyr Ser Ser Gly Asn), and SEQ ID NO: 16 (Cys Ala Met Ser Asp Thr Gly Asn Gln Phe Tyr Phe); and a beta chain comprising the CDR sequences shown in SEQ ID NO: 17 (Pro Arg His Asp Thr), SEQ ID NO: 18 (Phe Tyr Glu Lys Met Gln), and SEQ ID NO: 19 (Cys Ala Ser Ser Phe Arg Gly Gly Gly Ala Asn Val Leu Thr Phe);
and wherein the total number of T cells comprised in the composition is from about 0.5×10⁷ T cells to about 1×10¹⁰ T cells.

29. The pharmaceutical composition of claim 28, wherein the total number of T cells comprised in the composition is about 0.75×1×10⁸ T cells, about 1×10⁸ cells, about 1×10⁹ T cells, about 3×10⁹ T cells, about 4×10⁹ T cells, about 5×10⁹ T cells, about 6×10⁹ T cells, about 7×10⁹ T cells, about 8×10⁹ T cells or about 9×10⁹T cells.

30. A diagnostic immunostaining kit for selecting a patient for treatment of a solid tumor, wherein cells of the tumor express the human melanoma associated antigen 1 (MAGE-A1) (e.g., UniProtKB accession number P43355 (MAGA1_HUMAN), wherein the kit comprises
the monoclonal IgG1 mouse antibody MA454 or an antigen binding fragment of the antibody MA454, and
a secondary antibody capable of binding to the monoclonal IgG1 mouse antibody MA454.

31. The kit of claim 30, wherein the secondary antibody comprises an optically detectable label.
